(19) United States
(12) Patent Application Publication
(10) Pub. No.: US 2017/0058029 A1
(43) Pub. Date: Mar. 2, 2017

(54) HUMAN ANTIBODY-PRODUCING CELL

(71) Applicant: Chiome Bioscience Inc., Tokyo (JP)

(72) Inventors: Shuichi Hashimoto, Tokyo (JP); Tomoaki Uchiki, Tokyo (JP); Shigeshisa Kawata, Tokyo (JP); Kenjiro Asagoshi, Tokyo (JP); Takashi Yabuki, Tokyo (JP); Hitomi Sano, Tokyo (JP); Shunsuke Miyai, Tokyo (JP); Naoki Takahashi, Tokyo (JP); Aki Takesue, Tokyo (JP); Atsushi Sawada, Tokyo (JP)

(21) Appl. No.: 15/308,085

(22) PCT Filed: May 1, 2015
(86) PCT No.: PCT/JP2015/063090

§ 371 Date: Oct. 31, 2016

(30) Foreign Application Priority Data
May 2, 2014 (JP) 2014-095236

(51) Int. Cl. C07K 16/28; C07K 16/24; C12N 5/0781; C07K 16/18

(52) U.S. Cl. CPC C07K 16/28; C07K 16/18; C07K 16/244; C12N 5/0635; C07K 2317/14; C07K 2317/21; C07K 2317/515; C07K 2317/51; C07K 2317/565; C12N 2510/02

(57) ABSTRACT

It is an object of the present invention to provide a chicken B cell that expresses a variety of human antibodies. The solution to the problem in the present invention is a chicken B cell, in which, in an antibody light chain gene locus thereof, all or a part of a DNA sequence derived from a human antibody light chain variable region and a human antibody light chain constant region are inserted, or the antibody light chain gene locus is replaced with all or a part of a DNA sequence derived from a human antibody light chain variable region and a human antibody light chain constant region, and in an antibody heavy chain gene locus thereof, all or a part of a DNA sequence derived from a human antibody heavy chain variable region and a human antibody heavy chain constant region are inserted, or the antibody heavy chain gene locus is replaced with all or a part of a DNA sequence derived from a human antibody heavy chain variable region and a human antibody heavy chain constant region, and in an antibody light chain pseudogene locus thereof, two or more DNA sequences derived from human antibody light chain variable regions are inserted, or the antibody light chain pseudogene locus is replaced with two or more DNA sequences derived from human antibody light chain variable regions, and/or in an antibody heavy chain pseudogene locus thereof, two or more DNA sequences derived from human antibody heavy chain variable regions are inserted, or the antibody heavy chain pseudogene locus is replaced with two or more DNA sequences derived from human antibody heavy chain variable regions.

### TECHNICAL FIELD

The present invention relates to a cell that produces a human antibody. More specifically, the present invention relates to a chicken B cell that produces a human antibody.

### BACKGROUND ART

An antibody is useful for performing identification of biological substances, the function analysis thereof, and the like, and it also plays an important role in the treatment of diseases. Such an antibody binds to a specific antigen in a living body and provokes various in vivo protective immune reactions, such as antibody-dependent cell-mediated cytotoxicity (ADCC) or complement-dependent cytotoxicity (CDC), so that the antibody recognizes a “foreign substance” to a living body and removes it. Paying attention on the ability of such an antibody, in recent years, antibody drugs have been vigorously developed. In order to treat a wide variety of diseases, it is necessary to promptly and simply supply antibodies that recognize various target antigens.

To date, as a means for preparing an antibody group comprising desired physical and physiological characteristics, a technique of producing a monoclonal antibody has been used. In a case where such a monoclonal antibody is produced, a hybridoma method comprising fusing B cells generated as a result of in vivo immunity with myelomas has been generally applied. However, this method has many problems in that, for example, immunological tolerance is produced due to the use of in vivo immunity, and it takes time and effort to finally obtain a desired antibody.

As a technique of overcoming the problem regarding immunological tolerance, a method that does not utilize in vivo immunity has been developed. This method comprises embedding a single chain valuable fragment (scFv) gene consisting of the variable regions of various antibodies into a phage particle, then allowing the phage to present a single chain antibody gene product on the surface thereof, and then obtaining a clone having an affinity for an antigen of interest from a single chain antibody library displayed by this phage. This method is referred to as a phage display method. This technique is excellent in terms of avoidance of immunological tolerance. However, since the clone obtained from the phage library is a single chain clone, it is necessary to prepare a complete antibody consisting of two chains according to a recombinant DNA technique or the like. Moreover, a change may be generated in affinity during the process of converting a single chain antibody to a complete antibody, and thus, there may be a considerable number of cases where a variable region sequence needs to be adjusted. Accordingly, in terms of time and effort, it cannot be said that the phage display method has been significantly improved in comparison to the method involving in vivo immunity.

As methods for overcoming the problem regarding the aforementioned existing method for producing a monoclonal antibody, an ADLib system (Patent Literature 1 and Non Patent Literature 1) and a method of further modifying such an ADLib system (Patent Literature 2) have been reported. The ADLib system is a technique capable of simply preparing a variety of antibodies with desired binding properties to all types of antigens. This method is a technique of selectively obtaining a desired antibody from an antibody library constructed from a chicken B cell-derived cell line DT40, in which antibody genes have become diversified autonomously. The ADLib system is superior to prior arts, in that it enables the avoidance of immunological tolerance, which is an advantage of an in vitro system antibody production technique, and in that a complete IgM antibody can be promptly obtained.

In general, in a case where an antibody is administered as a pharmaceutical product to an animal or a human, the antibody is required to be compatible with the type of the animal species, while considering minimization of immunogenicity in vivo. Hence, utilizing a DNA recombination technique, a chimeric antibody has been produced by replacing the constant region of a monoclonal antibody produced in mice or the like with a humanized constant region, and a humanized antibody has also been produced by transplanting the complementarity determining region (CDR) of the antibody into a human antibody. However, there may be a case where a change would occur in affinity or function in the process of such chimerization or humanization therefore it cannot be said that the probability of producing a humanized antibody that retains the original functions is high. Moreover, such a chimeric antibody or a humanized antibody only has a small region derived from other animal species therefore its complementarity determining region has immunogenicity, although its immunogenicity is lower than that of the original monoclonal antibody. Hence, such a chimeric antibody or a humanized antibody has been problematic in terms of the appearance of anti-antibody.

Thus, it has been attempted to produce an animal generating a human antibody by introducing a human antibody gene into a mouse. In Patent Literature 3, a microcell of a chromosomal fragment comprising a human antibody gene has been transplanted into mouse ES cells, and then, a chimeric mouse has been produced from the ES cells, thereby producing a chimeric mouse that expresses a human antibody. Moreover, in Non Patent Literature 2, genomic DNA in a mouse antibody gene locus has been replaced with the corresponding genomic DNA in a human antibody gene locus, so as to produce a chimeric mouse that expresses a human antibody. By both of the methods, it is possible to obtain an antibody having complementarity determining regions that are completely derived from human antibody genes. However, since in vivo immunity is utilized in both of the methods, the problems regarding time and effort required until a desired antibody is finally obtained have still remained.

Under the aforementioned circumstances, even in the case of using the ADLib system, a method of producing an antibody having an amino acid sequence derived from a human from chicken B cells is necessary to prepare an antibody drug. In addition, in order to treat various diseases, a technique of simply producing an antibody group having various antigen-recognizing properties is necessary.

Nevertheless, differing from a case where a human antibody gene is introduced into a mouse to “humanize” it, in a case where chicken B cells are “humanized,” a difference in the system of diversifying an antibody becomes problematic. In the case of animals such as a chicken, a rabbit, a bovine or sheep, there is a region in which “pseudogenes” are gathered, upstream of an antibody variable region. The sequence of the respective pseudogenes are similar to that of antibody variable region and are not translated by themselves. All or a part of variable region sequences are rewritten by pseudogene sequences by a phenomenon referred to as gene conversion, so that a variety of sequences are generated. Diversification of antibodies occurs as a result of V(D)J recombination in animals such as a human or a mouse. In contrast, in animals such as a chicken, a rabbit, a bovine or sheep, such diversification of antibodies occurs by a mechanism referred to as gene conversion that is completely different from the recombination. Moreover, since the pseudogene region has a structure comprising an extremely large number of repeating sequences, it is significantly difficult to analyze the sequence of this region. Thus, even at the time point of March 2015, the pseudogene region of a chicken antibody heavy chain has not yet been analyzed. Due to such a difference in mechanisms or a problem regarding the structure of an antibody gene locus, it has been considered extremely difficult to construct a system of producing a desired human antibody from chicken B cells.

Under such circumstances, it has been reported that a cell line was produced by replacing a variable region and a pseudogene region present in the antibody gene locus of DT40 cells with human-derived sequences (Non Patent Literature 3). In this publication, a chicken antibody variable region has been replaced with a gene sequence comprising fluorescent proteins, namely, a green fluorescent protein (GFP) and a cyan fluorescent protein (CFP), and an attP sequence as a recombinase-recognizing site, and a DT40 cell, into which a human antibody variable region and the after-mentioned pseudogene sequence had been inserted utilizing the recombinase, has been then produced, and thereafter, the occurrence of gene conversion has been confirmed (Non Patent Literatures 3 and 4). However, since a chicken-derived constant region sequence has been used, the expressed antibody molecule is a human-chicken chimeric antibody, and thus, the immunogenicity of the antibody is extremely high. Furthermore, since the light chain is a fusion protein of a human-derived κ chain and a chicken-derived λ chain, the antibody molecule is largely different from a native human antibody. Hence, in order to produce an antibody used for the treatment of humans, it is necessary to further carry out a chimerization operation, and it is still likely that affinity or functions will be changed in the process of chimerization or humanization. Further, with regard to the sequence of the pseudogene region used herein, in the case of a light chain, a minimalist library that is a cluster of artificial sequences, in which one amino acid in each of the CDR sequences of the inserted human antibody variable region has been substituted with Tyr or Trp, and the sequences in which mutations have been introduced into the framework region of a variant of a naturally existing human antibody variable region, have been utilized. On the other hand, a heavy chain thereof is composed of an artificial sequence in which each of the CDR sequences of the inserted human antibody variable region has been substituted with Tyr or Trp, and mutant sequences thereof. These sequences have been designed for the purpose of allowing a small number of pseudogenes to correspond to a variety of antigens. A method of obtaining an antigen-specific antibody in the cells has not been reported.

### CITATION LIST

### Patent Literature

Patent Literature 1: Japanese Patent No. 4214234
Patent Literature 2: WO 2008/047480
Patent Literature 3: WO 97/07671

### Non Patent Literature

Non Patent Literature 1: Seo et al., Nature Biotech, Volume 23, pp. 731-735, 2005
Non Patent Literature 2: Recombinant Antibodies for Immunotherapy, pp. 100-108, 2009, Cambridge Press
Non Patent Literature 3: Schusser et al., PLOS ONE, Volume 8 issue 11 e80108, 2013
Non Patent Literature 4: Leighton et al., Frontiers in Immunology, Volume 6 Article 126, 2015

### SUMMARY OF INVENTION

### Technical Problem

Under the aforementioned circumstances, it is an object of the present invention to provide chicken B cells, each of which produces each of various human antibodies.

It is another object of the present invention to provide a method for producing antibodies from the chicken B cells.

### Solution to Problem

The present inventors have conducted intensive studies regarding production of cells that produce a variety of human antibodies. As a result, the inventors have inserted or replaced the genes in the variable region and constant region of the light chain and heavy chain of a human antibody into chicken B cell antibody light chain and heavy chain gene loci, so that they have succeeded in obtaining transformed cells that each express a human antibody. Moreover, the present inventors have further produced transformed cells, in which a plurality of human-derived antibody variable region sequences have been inserted as pseudogenes into the light chain and heavy chain, and thereafter, the inventors have treated these cells with an HDAC inhibitor, so that they have confirmed that gene conversion has occurred in the transformed cells, as in the case of the original chicken B cells. Furthermore, the present inventors have also succeeded in producing an antigen-specific human antibody from the transformed cells.

That is to say, the present inventors have confirmed that the aforementioned cells are prepared, and from each of the prepared cell, antibody in which the variable region has been modified in various ways can be obtained, and they have then succeeded in producing a variety of human antibodies, thereby completing the present invention.

Specifically, the present invention includes the following (1) to (21):

(1) A chicken B cell, in which, in an antibody light chain gene locus thereof, all or a part of a DNA sequence derived from a human antibody light chain variable region and a human antibody light chain constant region are inserted, or the antibody light chain gene locus is replaced with all or a part of a DNA sequence derived from a human antibody light chain variable region and a human antibody light chain constant region, and in an antibody heavy chain gene locus thereof, all or a part of a DNA sequence derived from a human antibody heavy chain variable region and a human antibody heavy chain constant region are inserted, or the antibody heavy chain gene locus is replaced with all or a part of a DNA sequence derived from a human antibody heavy chain variable region and a human antibody heavy chain constant region, and in an antibody light chain pseudogene locus thereof, two or more DNA sequences derived from human antibody light chain variable regions are inserted, or the antibody light chain pseudogene locus is replaced with two or more DNA sequences derived from human antibody light chain variable regions, and/or in an antibody heavy chain pseudogene locus thereof, two or more DNA sequences derived from human antibody heavy chain variable regions are inserted, or the antibody heavy chain pseudogene locus is replaced with two or more DNA sequences derived from human antibody heavy chain variable regions.
(2) The chicken B cell according to (1) above, wherein the number of the DNA sequences derived from the human antibody light chain variable regions to be inserted into the antibody light chain pseudogene locus, or to be replaced with the sequences in the antibody light chain pseudogene locus is 5 or more.
(3) The chicken B cell according to (1) above, wherein the number of the DNA sequences derived from the human antibody heavy chain variable regions to be inserted into the antibody heavy chain pseudogene locus, or to be replaced with the sequences in the antibody heavy chain pseudogene locus is 5 or more.
(4) The chicken B cell according to (1) above, wherein the number of the DNA sequences derived from the human antibody light chain variable regions to be inserted into the antibody light chain pseudogene locus, or to be replaced with the sequences in the antibody light chain pseudogene locus is 15 or more.
(5) The chicken B cell according to (1) above, wherein the number of the DNA sequences derived from the human antibody heavy chain variable regions to be inserted into the antibody heavy chain pseudogene locus, or to be replaced with the sequences in the antibody heavy chain pseudogene locus is 15 or more.
(6) The chicken B cell according to any one of (1) to (5) above, wherein the position of the DNA sequence derived from the human antibody light chain variable region inserted into the antibody light chain gene locus is upstream of the position of the DNA sequence derived from the human antibody light chain constant region, and the position of the DNA sequence derived from the human antibody heavy chain variable region inserted into the antibody heavy chain gene locus is upstream of the position of the DNA sequence derived from the human antibody heavy chain constant region.
(7) The chicken B cell according to (6) above, wherein the positions of the DNA sequence derived from the human antibody light chain variable region and the DNA sequence derived from the human antibody light chain constant region inserted into the antibody light chain gene locus are upstream of the positions of a chicken antibody light chain variable region and a constant region thereof, and the positions of the human antibody heavy chain variable region and constant region inserted into the antibody heavy chain gene locus are upstream of the positions of a chicken antibody heavy chain variable region and a constant region thereof.
(8) The chicken B cell according to any one of (1) to (7) above, wherein the human antibody heavy chain is a γ chain.
(9) The chicken B cell according to any one of (1) to (8) above, wherein the human antibody light chain is a γ chain or a κ chain.
(10) The chicken B cell according to any one of (1) to (9) above, which has an ability to express a human antibody on the cell surface thereof and also to secrete the human antibody into a culture solution.
(11) The chicken B cell according to any one of (1) to (10) above, which is a DT40 cell.
(12) The chicken B cell according to any one of (1) to (11), which has undergone to a treatment of relaxing chromatin.
(13) The chicken B cell according to (12) above, wherein the treatment of relaxing chromatin is reduction or deletion of the function of histone deacetylase in the chicken B cell.
(14) The chicken B cell according to (13) above, wherein the method for reducing or deleting the function of histone deacetylase is reduction or deletion of the expression of a histone deacetylase gene in the chicken B cell.
(15) The chicken B cell according to (14) above, wherein the histone deacetylase is HDAC2.
(16) The chicken B cell according to (13) above, wherein the method for reducing or deleting the function of histone deacetylase is a treatment with an HDAC inhibitor.
(17) An antibody-producing cell library consisting of the chicken B cells according to any one of (12) to (16) above.
(18) A method for producing an antibody from the chicken B cells according to any one of (1) to (16) above.
(19) An antibody obtained by the method according to (18) above.
(20) A kit for producing the chicken B cells according to any one of (1) to (16) above.
(21) A kit for producing an antibody by the method according to (18) above.

### Advantageous Effects of Invention

It becomes possible to simply and promptly obtain human antibodies that recognize a variety of antigens from the cells of the present invention.

### BRIEF DESCRIPTION OF DRAWINGS

FIG. 1 is a view schematically showing the structure of a targeting vector for replacing the antibody light chain constant region and antibody light chain variable region of a DT40 cell with the light chain constant region and light chain variable region of a human-derived antibody (IgG). cpV1 to cpV3: chicken pseudogenes.

FIG. 2 is a view schematically showing the structure of a targeting vector for inserting a sequence for confirming gene conversion (GC confirmation sequence; a human pseudogene consisting of a sequence derived from a human antibody variable region, the same applied below) downstream of the antibody light chain pseudogene region of a DT40 cell. cpV1 to cpV3: chicken pseudogenes.

FIG. 3 is a view schematically showing the structure of a targeting vector for deleting the antibody light chain pseudogene region of a DT40 cell, and replacing the antibody light chain pseudogene region of the DT40 cell with a GC confirmation sequence. cpV25, cpV1: chicken pseudogenes.

FIG. 4 is a view schematically showing the structure of a targeting vector for replacing the antibody heavy chain constant region and antibody heavy chain variable region of a DT40 cell with the heavy chain constant region and heavy chain variable region of a human-derived antibody (IgG) and also for inserting a GC confirmation sequence therein.

FIG. 5 is a view schematically showing the procedures for replacing the antibody light chain gene regions of a DT40 cell with the light chain variable region and light chain constant region of a human-derived antibody, and the thus obtained antibody light chain gene region of the DT40 cell. cpV1 to cpV3: chicken pseudogenes.

FIG. 6 is a view schematically showing the procedures for replacing the antibody light chain gene regions of a DT40 cell with human-derived antibody light chain variable region and light chain constant region genes, and further inserting a GC confirmation sequence downstream of the antibody light chain pseudogene region of the DT40 cell, and the obtained DT40 cell antibody light chain gene region. cpV1 to cpV3: chicken pseudogenes; and hpV1 and hpV2: human pseudogenes.

FIG. 7 is a view schematically showing the procedures for replacing the antibody light chain gene regions of a DT40 cell with human-derived antibody light chain variable region and light chain constant region genes, and further deleting the antibody light chain pseudogene region of the DT40 cell, and the thus obtained antibody light chain gene region of the DT40 cell. cpV25 and cpV1: chicken pseudogenes.

FIG. 8 is a view schematically showing the procedures for replacing the antibody light chain gene regions of a DT40 cell with human-derived antibody light chain variable region and light chain constant region genes, and further replacing the pseudogene region of the DT40 cell with a GC confirmation sequence, and the obtained DT40 cell antibody light chain gene region. cpV25 and cpV1: chicken pseudogenes; and hpV1 and hpV2: human pseudogenes.

FIG. 9 shows the results obtained by analyzing by flow cytometry an antibody produced by a cell line in which a human-derived antibody light chain gene region has been inserted into the antibody light chain gene region of a DT40 cell, or a cell line in which the antibody light chain gene region of a DT40 cell has been replaced with a human antibody light chain gene.

FIG. 10 is a view schematically showing the procedures for converting the antibody heavy chain gene regions of a DT40 cell to the heavy chain variable region and heavy chain constant region of a human-derived antibody, and the thus obtained antibody heavy chain gene region of the DT40 cell.

FIG. 11 is a view schematically showing the procedures for converting the antibody heavy chain gene regions of a DT40 cell to the heavy chain variable region and heavy chain constant region gene of a human-derived antibody, and further inserting a GC confirmation sequence downstream of the heavy chain pseudogene region of the DT40 cell, the thus obtained antibody light chain gene region of the DT40 cell, and the structure of the used targeting vector. hpV1 and hpV2: human pseudogenes.

FIG. 12 shows the results obtained by analyzing by flow cytometry an antibody produced by a cell line, in which the antibody heavy chain gene regions of a DT40 cell have been replaced with human-derived antibody heavy chain variable region and heavy chain constant region genes, and further, a GC confirmation sequence has been inserted downstream of the heavy chain pseudogene region of the DT40 cell.

FIG. 13 is a view schematically showing the procedures for replacing the antibody light chain pseudogene region of a DT40 cell with a pseudogene consisting of a sequence derived from a human Igγ variable region (human light chain pseudogene), the thus obtained antibody light chain region of the DT40 cell, and the structure of the used targeting vector.

FIG. 14 is a view schematically showing the structure of a targeting vector for replacing the antibody heavy chain constant region of a DT40 cell with the heavy chain gene region of human IgG₁.

FIG. 15 is a view schematically showing the structure of a targeting vector for introducing the heavy chain variable region of a human-derived antibody and five pseudogenes consisting of human Igγ variable region-derived sequences (human heavy chain pseudogenes) into the antibody heavy chain variable region of a DT40 cell.

FIG. 16 is a view schematically showing the procedures for replacing the antibody light chain gene regions of a DT40 cell with the light chain variable region and light chain constant region of a human-derived antibody, and then inserting human light chain pseudogene sequences (five sequences) therein, and the thus obtained antibody light chain gene region of the DT40 cell. cpV25 and cpV1: chicken pseudogenes.

FIG. 17 is a view schematically showing the procedures for introducing the heavy chain variable region and heavy chain constant region of a human-derived antibody into the antibody heavy chain gene regions of a DT40 cell, and then inserting human heavy chain pseudogene sequences (five sequences) therein, and the thus obtained antibody heavy chain gene region of the DT40 cell.

FIG. 18 shows the results obtained by analyzing by flow cytometry an antibody produced by a cell line, in which the antibody light chain gene region and antibody heavy chain gene region of a DT40 cell have been replaced with human-derived antibody genes, and human light chain pseudogene sequences (five sequences) have been then inserted therein.

FIG. 19 is a view schematically showing the procedures for replacing the antibody light chain gene regions of a DT40 cell with the light chain variable region and light chain constant region of a human-derived antibody, and then inserting human heavy chain pseudogene sequences (15 sequences) therein, and the structure of the used targeting vector. pV25 and pV1: chicken pseudogenes.

FIG. 20 is a view schematically showing the procedures for introducing the heavy chain variable region and heavy chain constant region of a human-derived antibody into the antibody heavy chain gene regions of a DT40 cell, and then inserting human heavy chain pseudogene sequences (15 sequences) therein, and the structure of the used targeting vector.

FIG. 21 shows the results obtained by analyzing by flow cytometry an antibody produced by an antibody produced by an L15/H15 cell line, in which the antibody light chain gene region and antibody heavy chain gene region of a DT40 cell have been replaced with the gene regions of a human-derived antibody, and human light chain pseudogene sequences (15 sequences) have been then inserted therein.

FIG. 22 shows the results shown in FIG. 21, in which incorrect data have been corrected based on the data before the priority date.

FIG. 23 is a view schematically showing the procedures for replacing the antibody heavy chain variable region of a DT40 cell with the heavy chain variable region of a human-derived antibody, and at the same time, inserting a cassette sequence for RMCE into the antibody heavy chain variable region of the DT40 cell, and the structure of the used targeting vector.

FIG. 24 is a view schematically showing the procedures for inserting 30 human heavy chain pseudogene sequences into the antibody heavy chain gene region of a DT40 cell, and the structure of the used targeting vector.

FIG. 25 shows the results obtained by analyzing by flow cytometry an antibody produced by an L15/H30 cell line, in which the antibody light chain gene region and antibody heavy chain gene region of a DT40 cell have been replaced with the gene regions of a human-derived antibody, and thereafter, 15 human light chain pseudogene sequences have been inserted into the light chain and 30 human heavy chain pseudogene sequences have been inserted into the heavy chain.

FIG. 26 is a view schematically showing the procedures for inserting 30 human light chain pseudogene sequences into the antibody light chain gene region of a DT40 cell, and the structure of the used targeting vector.

FIG. 27 shows the results obtained by analyzing by flow cytometry an antibody produced by an L30/H30 cell line, in which the antibody light chain gene region and antibody heavy chain gene region of a DT40 cell have been replaced with the gene regions of a human-derived antibody, and thereafter, 30 human light chain pseudogene sequences have been inserted into the light chain and 30 human heavy chain pseudogene sequences have been inserted into the heavy chain.

FIG. 28 shows the results obtained by analyzing by flow cytometry an antibody produced by an L30/H15 cell line, in which the antibody light chain gene region and antibody heavy chain gene region of a DT40 cell have been replaced with the gene regions of a human-derived antibody, and thereafter, 30 human light chain pseudogene sequences have been inserted into the light chain and 15 human heavy chain pseudogene sequences have been inserted into the heavy chain.

FIG. 29 is a view schematically showing the procedures for additionally inserting 15 human heavy chain pseudogene sequences into the antibody heavy chain gene region of the L30/H15 cell line in the forward or reverse direction according to an RMCE method, so as to have 30 human pseudogene sequences in total, and the structure of the used targeting vector. In the figure, the symbols “for” and “rev” indicate the forward direction and the reverse direction, respectively. The same is applied to other figures below.

FIG. 30 shows the results obtained by analyzing by flow cytometry an antibody produced by a cell line, in which 15 human heavy chain pseudogene sequences have been additionally inserted into the L30/H15 cell line in the forward or reverse direction according to the RMCE method (which is an L30/H15f15f cell line in the case of the forward direction, and an L30/H15r15f cell line in the case of the reverse direction).

FIG. 31 is a view schematically showing the procedures for additionally inserting 15 human heavy chain pseudogene sequences to each of the antibody heavy chain gene regions of the L30/H15f15f cell line and the L30/H15r15f cell line in the forward or reverse direction, so as to have 45 human pseudogene sequences in total, and the structure of the used targeting vector.

FIG. 32 shows the results obtained by analyzing by flow cytometry an antibody produced by a cell line (L30/H15f15f15f cell line), in which 15 human heavy chain pseudogene sequences have been additionally inserted into the antibody heavy chain gene region of the L30/H15f15f cell line in the forward direction.

FIG. 33 shows the results obtained by analyzing by flow cytometry an antibody produced by a cell line (L30/H15r15r15f cell line), in which 15 human heavy chain pseudogene sequences have been additionally inserted into the antibody heavy chain gene region of the L30/H15r15f cell line in the reverse direction.

FIG. 34 is a view schematically showing the procedures for additionally inserting 15 human heavy chain pseudogene sequences into each of the antibody heavy chain gene regions of the L30/H15f15f15f cell line and the L30/H15r15r15f cell line in the forward or reverse direction according to the RMCE method, so as to have 60 human pseudogene sequences in total, and the structure of the used targeting vector.

FIG. 35 shows the results obtained by analyzing by flow cytometry an antibody produced by a cell line, in which 30 human light chain pseudogene sequences have been inserted into the antibody light chain gene region of a DT40 cell, and 60 human heavy chain pseudogene sequences have been inserted into the antibody heavy chain gene region thereof.

FIG. 36 shows the results obtained by separating a cell population binding to a Plexin A4 protein from a TSA-treated L15/H15 cell line using an ADLib system, and then analyzing the cell population by flow cytometry.

FIG. 37 shows the results obtained by performing single cell sorting on a cell population in the P5 area of FIG. 36, plating the obtained cells on a 96-well plate to culture them, and carrying out ELISA analysis on the cell supernatant in each well. The horizontal axis indicates the number of each well fraction of the 96-well plate, from which the cell supernatant used as a measurement sample is derived. Trypsin inhibitor: trypsin inhibitor; SA: streptavidin; OA: ovalbumin.

FIG. 38 shows the results obtained by analyzing antibodies secreted from positive clones #62 and #20 according to Western blotting. The samples were treated under reduction and non-reduction conditions, and Western blotting was then carried out on the samples, using a goat anti-human IgGγ antibody.

FIG. 39 shows the results obtained by analyzing antibodies secreted from positive clones #62 and #20 according to Western blotting. The samples were treated under reduction and non-reduction conditions, and Western blotting was then carried out on the samples, using a goat anti-human IgGγ antibody.

FIG. 40 shows the results obtained by analyzing antibodies secreted from positive clones #62 and #20 according to Western blotting. The samples were treated under reduction and non-reduction conditions, and Western blotting was then carried out on the samples, using a goat anti-chicken IgM antibody.

FIG. 41 shows the results of a primary screening of a cell population binding to His-AP-hSema3A from the TSA-treated L15/H15 cell line, which has been carried out using an ADLib system.

FIG. 42 shows the results of a secondary screening, which has been carried out after the monocloning of the cell population binding to His-AP-hSema3A.

FIG. 43 shows the results obtained by analyzing by Western blotting a His-AP-hSema3A antibody secreted from the positive clones after completion of the secondary screening. The samples were treated under reduction and non-reduction conditions, and Western blotting was then carried out on the samples, using a goat anti-human IgGγ antibody or a goat anti-human IgG antibody.

FIG. 44 shows the results of a primary screening of a cell population binding to IL-8 from a TSA-treated L30/H45 cell line, which has been carried out using an ADLib system.

FIG. 45 shows the results of a secondary screening, which has been carried out after the monocloning of the cell population binding to IL-8.

FIG. 46 shows the results obtained by analyzing by Western blotting an IL-8 antibody secreted from the positive clones after completion of the secondary screening. The samples were treated under reduction and non-reduction conditions, and Western blotting was then carried out on the samples, using a goat anti-human IgGγ antibody or a goat anti-human IgGλ antibody.

### DESCRIPTION OF EMBODIMENTS

One embodiment of the present invention is a chicken B cell, in which, in an antibody light chain gene locus thereof, all or a part of a DNA sequence derived from a human antibody light chain variable region and a human antibody light chain constant region are inserted, or the antibody light chain gene locus is replaced with all or a part of a DNA sequence derived from a human antibody light chain variable region and a human antibody light chain constant region, and in an antibody heavy chain gene locus thereof, all or a part of a DNA sequence derived from a human antibody heavy chain variable region and a human antibody heavy chain constant region are inserted, or the antibody heavy chain gene locus is replaced with all or a part of a DNA sequence derived from a human antibody heavy chain variable region and a human antibody heavy chain constant region, and in an antibody light chain pseudogene locus thereof, two or more DNA sequences derived from human antibody light chain variable regions are inserted, or the antibody light chain pseudogene locus is replaced with two or more DNA sequences derived from human antibody light chain variable regions, and/or in an antibody heavy chain pseudogene locus thereof, two or more DNA sequences derived from human antibody heavy chain variable regions are inserted, or the antibody heavy chain pseudogene locus is replaced with two or more DNA sequences derived from human antibody heavy chain variable regions.

The B cell of the present invention is an antibody-producing B cell derived from a chicken, and an example of the B cell of the present invention can be a DT 40 cell from a chicken. Other than such DT40 cells, B cells from a bovine, sheep, a rabbit and the like, which have a system for diversifying antibody sequences according to gene conversion, can also be used.

Moreover, in addition to the aforementioned configuration such that, in an antibody light chain gene locus thereof, all or a part of a DNA sequence derived from a human antibody light chain variable region and a human antibody light chain constant region are inserted, or the antibody light chain gene locus is replaced with all or a part of a DNA sequence derived from a human antibody light chain variable region and a human antibody light chain constant region, and in an antibody heavy chain gene locus thereof, all or a part of a DNA sequence derived from a human antibody heavy chain variable region and a human antibody heavy chain constant region are inserted, or the antibody heavy chain gene locus is replaced with all or a part of a DNA sequence derived from a human antibody heavy chain variable region and a human antibody heavy chain constant region, and in an antibody light chain pseudogene locus thereof, two or more DNA sequences derived from human antibody light chain variable regions are inserted, or the antibody light chain pseudogene locus is replaced with two or more DNA sequences derived from human antibody light chain variable regions, and/or in an antibody heavy chain pseudogene locus thereof, two or more DNA sequences derived from human antibody heavy chain variable regions are inserted, or the antibody heavy chain pseudogene locus is replaced with two or more DNA sequences derived from human antibody heavy chain variable regions, the chicken B cell of the present invention may also comprise mutations or modifications in other gene regions and the like.

The term “antibody light chain gene locus” is used herein to mean a gene locus in which a gene encoding the light chain variable region and light chain constant region of an antibody is present, and the term “antibody heavy chain gene locus” is a gene locus in which a gene encoding the heavy chain variable region and heavy chain constant region of an antibody is present.

In addition, the term “pseudogene” is used herein to mean a DNA sequence that is similar to a functional gene but does not function as an expressing gene. The chicken antibody light chain pseudogene locus and the chicken heavy chain pseudogene locus are each located upstream of the antibody light chain gene locus and heavy chain gene locus, in which the functional gene is present, and contribute to generate the diversity as a result of gene conversion. A pseudogene is not present in an antibody gene locus in human genome, but in the present description, a DNA sequence having a sequence similar to a human antibody variable region, which has been introduced into a chicken antibody gene locus for the purpose of causing gene conversion with the inserted human antibody variable region, is collectively referred to as a “human pseudogene.”

A DNA sequence derived from a human antibody light chain variable region and a human antibody light chain constant region, which have been inserted into a chicken antibody light chain gene locus or have been replaced with a sequence in the chicken antibody light chain gene locus, may be all of the sequence of the human antibody light chain variable region and the human antibody light chain constant region, or may also be a part of the sequence. Moreover, the position of the DNA sequence derived from the human antibody light chain variable region inserted into the chicken antibody light chain gene locus is desirably upstream of a position, into which the DNA sequence derived from the human antibody light chain constant region has been inserted.

Likewise, a DNA sequence derived from a human antibody heavy chain variable region and a human antibody heavy chain constant region, which have been inserted into a chicken antibody heavy chain gene locus or have been replaced with a sequence in the chicken antibody heavy chain gene locus, may be all of the sequence of the human heavy chain variable region and the human heavy chain constant region, or may also be a part of the sequence. Moreover, the position of the DNA sequence derived from the human antibody heavy chain variable region inserted into the chicken antibody heavy chain gene locus is desirably upstream of a position, into which the DNA sequence derived from the human antibody heavy chain constant region has been inserted.

Furthermore, the positions of the DNA sequences derived from the human antibody heavy chain variable region and the human antibody constant region inserted into the chicken antibody light chain gene locus are desirably upstream of the positions of DNA sequence derived from a chicken antibody light chain variable region and a chicken antibody light chain constant region. Likewise, the positions of the DNA sequences derived from the human antibody heavy chain variable region and the human antibody heavy chain constant region inserted into the chicken antibody heavy chain gene locus are desirably upstream of the positions of the chicken antibody heavy chain variable region and the chicken antibody heavy chain constant region.

In the present embodiment, the DNA sequences derived from the human antibody light chain variable region, which have been inserted into the chicken antibody light chain pseudogene locus or have been replaced with the DNA sequences in the chicken antibody light chain pseudogene locus (which are also referred to as “human light chain pseudogene sequences” in the present description and drawings) can be the sequences of known human antibody light chain variable regions. Such sequence information can also be obtained from websites such as V Base (http://www2.mrc-lmb.cam.ac.uk/vbase/list2.php). As such human light chain pseudogene sequences, DNA sequences comprising all or a part of the CDR1, CDR2 and CDR3 of the human antibody light chain variable region can be used. It is desirable that each CDR be not identical to the DNA sequence of the human antibody light chain variable region that has been inserted into the chicken antibody light chain gene locus or has been replaced with the sequence in the chicken antibody light chain gene locus, or to the CDRs of other human light chain pseudogene sequences. Moreover, it is desirable that the framework (FW) region of the human light chain pseudogene sequence be identical to that of the human antibody light chain variable region that has been inserted into the chicken antibody light chain gene locus or has been replaced with the sequence in the chicken antibody light chain gene locus. For instance, when the DNA sequence of SEQ ID NO: 5 is selected as an antibody light chain variable region, the sequences shown in SEQ ID NO: 69 to SEQ ID NO: 88 and SEQ ID NO: 127 to SEQ ID NO: 141 can be used in combination as a light chain pseudogene.

Likewise, the DNA sequences derived from the human antibody heavy chain variable regions, which have been inserted into the chicken antibody heavy chain pseudogene locus or have been replaced with the DNA sequences in the chicken antibody heavy chain pseudogene locus (which are also referred to as “human heavy chain pseudogene sequences” in the present description and drawings) can be the sequences of known human antibody heavy chain variable regions. Such sequence information can also be obtained from websites such as V Base (http://www2.mrc-lmb.cam.ac.uk/vbase/list2.php). As such human heavy chain pseudogene sequences, DNA sequences comprising all or a part of the CDR1, CDR2 and CDR3 of the human antibody heavy chain variable regions can be used. It is desirable that each CDR be not identical to the DNA sequence of the human antibody heavy chain variable region that has been inserted into the chicken antibody heavy chain gene locus or has been replaced with the sequence in the chicken antibody heavy chain gene locus, or to the CDRs of other human heavy chain pseudogene sequences. Moreover, it is desirable that the framework (FW) regions of the human heavy chain pseudogene sequences be identical to that of the human antibody heavy chain variable region that has been inserted into the chicken antibody heavy chain gene locus or has been replaced with the sequence in the chicken antibody heavy chain gene locus. For instance, when the DNA sequence of SEQ ID NO: 30 is selected as an antibody heavy chain variable region, the sequences shown in SEQ ID NO: 89 to SEQ ID NO: 108, SEQ ID NO: 127 to SEQ ID NO: 141, SEQ ID NO: 147 to SEQ ID NO: 161, and SEQ ID NO: 165 to SEQ ID NO: 179 can be used in combination as a heavy chain pseudogene.

In the present embodiment, the position of the DNA sequences derived from the human antibody light chain variable regions inserted into the chicken antibody light chain pseudogene locus is desirably upstream of the position of the human antibody light chain variable region inserted into the chicken antibody light chain gene locus. In addition, when a chicken antibody light chain pseudogene sequence remains, the DNA sequences derived from the human antibody light chain variable regions is desirably located downstream of the chicken antibody light chain pseudogene region. Likewise, the position of the DNA sequences derived from the human antibody heavy chain variable regions inserted into the chicken antibody heavy chain pseudogene locus is desirably upstream of the position of the human antibody heavy chain variable region inserted into the chicken antibody heavy chain gene locus. In addition, when a chicken antibody heavy chain pseudogene sequence remains, the DNA sequence derived from the human antibody heavy chain variable regions is desirably located downstream of the chicken antibody heavy chain pseudogene region.

Moreover, in the present embodiment, the DNA sequences derived from the human antibody variable regions, which are inserted or substituted as a pseudogene upstream of the chicken antibody variable region, may be used either in the same direction as, or in the direction opposite to, the direction of the human antibody variable region sequence inserted or substituted into the chicken antibody variable region.

The number of the DNA sequences derived from the human antibody light chain variable regions, which are inserted into the chicken antibody light chain pseudogene locus, or are substituted (replaced) with pseudogenes in the chicken antibody light chain pseudogene locus, is preferably 2 or more, more preferably 5 or more, 7 or more, 9 or more, 11 or more, 13 or more, further preferably 15 or more, 20 or more, or 25 or more. Likewise, the number of DNA sequences derived from the human antibody heavy chain variable regions, which are inserted into the chicken antibody heavy chain pseudogene locus, or are substituted (replaced) with pseudogenes in the chicken antibody heavy chain pseudogene locus, is preferably 2 or more, more preferably 5 or more, 7 or more, 9 or more, 11 or more, 13 or more, further preferably 15 or more, 20 or more, 25 or more, and most preferably 30 or more.

It is to be noted that the DNA sequence derived from the human antibody light chain variable region or the DNA sequence derived from the human antibody heavy chain variable region, which is used herein, is considered to be one DNA sequence, when it is a series of continuous variable region homologous sequences contained in a region over a variable region, and comprises any one of FW1, CDR1, FW2, CDR2, FW3, CDR3 and FW4.

In the present embodiment, the type of a human antibody heavy chain, which is inserted into the chicken antibody heavy chain gene locus, may be any one of a γ chain, an s chain, an α chain, a β chain and a ε chain. It is preferably a γ chain. On the other hand, the type of a human antibody light chain, which is inserted into the chicken antibody light chain gene locus and the chicken antibody light chain pseudogene locus may be either a λ chain or a κ chain.

In the present embodiment, in order to use the transgenic chicken B cell for preparing an antibody library, the chicken B cell preferably does not only express a human antibody on the cell membrane thereof, but it also has an ability to secrete the human antibody into a culture solution. Therefore, when the introduced human antibody gene is transcribed, alternative splicing similar to an in vivo expression control mechanism needs to appropriately take place. Thus, with regard to introduction of the constant region, it is ideal to use a genomic structure that is completely identical to the constant region of a chicken antibody heavy chain constant region. However, since the nucleotide sequence of the genomic region of a chicken has not yet been analyzed at the time point of March 2015, this cannot be used. In the present embodiment, an intron sequence in the genomic sequence of a mouse antibody heavy chain has been used, and as a result, a human antibody on the surface of a cell membrane and a secretory human antibody have been successfully expressed at the same time. Accordingly, it is also possible to use, as a substitute, an intron sequence derived from known mammalian animals such as a mouse or a human.

In the present embodiment, a knocking-in method involving homologous recombination is used as a method of inserting a human antibody gene into a chicken antibody gene locus and/or replacing the gene in the chicken antibody gene locus with a human antibody gene. Moreover, as methods of inserting human antibody variable region-derived sequences into the pseudogene region in the chicken antibody chain gene locus and/or replacing the gene in the chicken antibody chain gene locus with a human antibody gene, a knocking-in method involving homologous recombination and a recombinase mediated cassette exchange (RMCE) method involving site-specific recombination can be preferably used.

As described above, a chicken B cell produced in the present embodiment means a chicken B cell, into which DNA sequences derived from a human antibody gene locus have been inserted, or which have been replaced with the DNA sequences derived from the human antibody gene locus; and a chicken B cell, in which a wide variety of mutations have been further introduced into the human-derived antibody light chain or heavy chain variable region of the aforementioned cell. Diversification of antibodies can be achieved by reorganization of the variable region that will cause generation of various variable region sequences. Therefore, a chicken B cell produced in the present embodiment also means a chicken B cell on which a treatment of introducing various mutations necessary for the reorganization of the variable region has been performed. Herein, examples of the method of introducing a mutation necessary for the reorganization of the variable region, which can be used herein, include methods known in the present technical field, such as a method of using B cells in which XRCC2 and XRCC3 have been deleted (e.g., Cumber et al., Nature Biotech. 204: 1129-1134 2002, JP Patent Publication (Kokai) No. 2003-503750 A, etc.), a method of controlling the expression of an AID gene (e.g., Kanayama et al., Nucleic Acids Res. 34: e10, 2006, JP Patent Publication (Kokai) No. 2004-298072 A, etc.), and a method of relaxing chromatin to promote gene conversion (e.g., Patent Literature 1, Patent Literature 2, Non Patent Literature 1, etc.). Among others, a method of relaxing chromatin to promote gene conversion is particularly preferable.

An example of the method of relaxing chromatin in a chicken B cell can be a method, which comprises inhibiting histone deacetylase (HDAC) activity existing in the chicken B cell by a certain method, and thereby significantly promoting gene conversion in the cell (for details, see the above-mentioned Patent Literature 1, Patent Literature 2, Non Patent Literature 1, etc.). As methods of inhibiting the activity of HDAC existing in cells, a method of treating the B cell of the present invention with an HDAC inhibitor (see Patent Literature 1 or Non Patent Literature 1), a method of reducing or deleting the function of an HDAC gene in a chicken B cell (see Patent Literature 2), and the like can be used. The HDAC inhibitor is not particularly limited, as long as it is able to inhibit the activity of HDAC. Examples of the HDAC inhibitor that can be used herein include trichostatin A (TSA), butyric acid, valproic acid, suberoylanilide hydroxamic acid (SAHA), CBHA (m-carboxycinnamic acid bis-hydroxamide), PTACH, Apicidin, Scriptaid, M344, APHA compound 8, BML-210, Oxamflatin, and MS-275. Other than these substances, an inactive-type protein of the HDAC as a target of activity inhibition (a dominant negative) may also be used as an inhibitory substance, if possible.

When the function of an HDAC gene is reduced or deleted, isoforms of the HDAC as a target whose gene function is to be reduced or deleted vary depending on an antibody-producing B cell used. The isoform is preferably an HDAC2 gene (for details, see Patent Literature 2).

The embodiment of the present invention that is relevant to the above-described embodiment includes an antibody-producing cell library consisting of the chicken B cells of the present invention. The present cell library means a population of the chicken B cells of the present invention that produce antibodies reacting with various antigens. The chicken B cell according to the above-described embodiment of the present invention is a cell, in which variation is generated in the variable region sequence of an antibody produced by the cell, by insertion of DNA sequences derived from a human-derived antibody variable regions into a chicken antibody pseudogene locus, etc., and the sequence of the antibody variable region is further diversified by a treatment of relaxing chromatin, so that the cell can have an ability to produce antibodies reacting with various antigens.

Therefore, a population comprising a plurality of the chicken B cells of the present invention can be a cell population producing antibodies reacting with various antigens.

Other embodiments of the present invention relate to a method for producing an antibody from the chicken B cell of the present invention, and a produced antibody.

The antibody produced from the chicken B cell of the present invention is a humanized antibody, and more preferably a human antibody. The humanized antibody means an antibody, in which a part of a sequence derived from a chicken gene is comprised in the amino acid sequence of the heavy chain or light chain of a produced antibody, and the human antibody means an antibody, in which the entire amino acid sequence of the heavy chain or light chain of a produced antibody is a sequence derived from a human gene.

When the chicken B cell of the present invention is cultured under culture conditions suitable for the cell, it produces a humanized antibody or a human antibody. It has been known that, in a cell line derived from chicken B cells such as DT40 cells, gene conversion occurs in an antibody gene locus thereof, although it does not occur with high efficiency. Thus, such gene conversion occurs also in the chicken B cell of the present invention, and primary variation in the produced antibodies is achieved by gene conversion occurring in an antibody gene locus, into which a human-derived antibody gene has been inserted. Moreover, by relaxing chromatin in the chicken B cell of the present invention, the gene conversion efficiency in the antibody gene locus is further increased, so that a population of chicken B cells capable of producing more various antibodies can be prepared. In order to select chicken B cells producing antibodies exhibiting desired antigen specificity by utilizing the relaxing of chromatin, an ADLib system can be used. The above-mentioned antibody-producing cell library can also be prepared by utilizing the ADLib system (see Patent Literature 1 or Non Patent Literature 1).

Furthermore, in particular, as a method of obtaining a desired antibody reacting with a membrane-bound protein, an ADLib axCELL (antigen expressing cell) method can be utilized (WO2010/064454).

Another embodiment of the present invention includes a kit for producing the chicken B cell of the present invention or producing an antibody.

The kit for producing the chicken B cell comprises: a medium, supplements and the like that are necessary for the culture of the chicken B cell; a vector used for inserting a DNA sequence derived from a human antibody gene into the antibody gene locus of the chicken B cell, or replacing the chicken B cell antibody gene locus with the DNA sequence derived from the human antibody gene; and reagents. In addition, the kit for producing the chicken B cell may also comprise: elements necessary for preparing an antibody-producing cell library, such as an HDAC inhibitor; and reagents necessary for reducing or deleting the function of the HDAC genes.

On the other hand, the kit for producing an antibody may comprise, as reagents for selecting an antibody having desired antigen specificity, all types of items that are considered necessary for production of the antibody. Examples of such reagents include various antigens, magnetic beads, a reagent for preparing such magnetic beads, a labeled antibody used to select an antibody, and a plate necessary for examinations such as ELISA, which are.

Hereinafter, the present invention will be described more in detail in the following examples. However, these examples are not intended to limit the scope of the present invention.

### Examples

### Confirmation of Gene Conversion

### 1. Preparation of Vectors

### 1-1. Replacement of Constant Region and Variable Region of Chicken Antibody Light Chain (Igλ) (Human LC KI Vector)

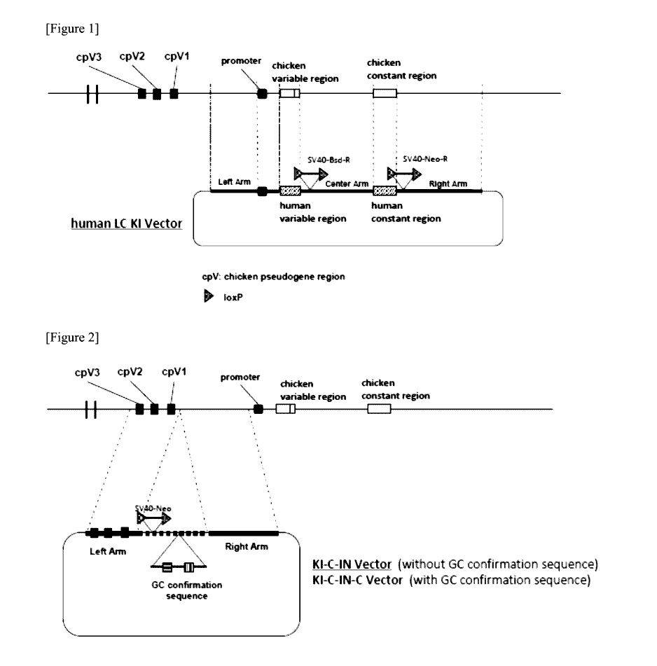

For replacement of the constant region and variable region of chicken Igλ, a targeting vector as shown in FIG. 1 was prepared. Methods for preparing individual parts will be described below.

### (1) Variable Region and Constant Region of Human Igλ

cDNA prepared from a human Burkitt's lymphoma cell line Ramos was used as a template, and PCR was carried out using a sense primer (TCCACCATGGCCTGGGCTCTG) (SEQ ID NO: 1) and an antisense primer (GTTGAGAACCTATGAACATTCTGTAGGGGCCAC) (SEQ ID NO: 2), so that the variable region and constant region of a human antibody light chain (Igλ) were amplified. The amplified regions were cloned into pGEM-T-Easy (Promega) to obtain pGEM-hIgG1-LC. This plasmid pGEM-hIgG1-LC was used as a template, and PCR was carried out using a sense primer (ATTGGCGCGCCTCTCCAGGTTCCCTGGTGCAGGCACAGTCTGCCCTGACTCAGC) (SEQ ID NO: 3) and an antisense primer (TTCCATATGAGCGACTCACCTAGGACGGTCAGCTTGGTCC) (SEQ ID NO: 4), so as to obtain the variable region (SEQ ID NO: 5) of human Igλ.

Moreover, the pGEM-hIgG1-LC was used as a template, and PCR was carried out using a sense primer (ATTGGCGCGCCTCTGCCTCTCTCTTGCAGGTCAGCCCAAGGCTGCCCCCTC) (SEQ ID NO: 6) and an antisense primer (GGAATTCCATATGGAGTGGGACTACTATGAACATTCTGTAGGGG) (SEQ ID NO: 7), so as to obtain the constant region (SEQ ID NO: 8) of human Igλ.

### (2) Left Arm

The genomic DNA of a chicken B cell line DT40 was used as a template, and PCR was carried out using a sense primer (GAGATCTCCTCCTCCCATCC) (SEQ ID NO: 9) and an antisense primer (CAAAGGACACGACAGAGCAA) (SEQ ID NO: 10), so that a region ranging from upstream of the variable region of chicken Igλ to downstream of the constant region thereof was amplified. A functional allele was separated from a non-functional allele by electrophoresis. The functional allele with a size of approximately 8.0 kbp was cut out, and it was then cloned into pGEM-T-Easy to obtain pGEM-cIgM-LCgenome. Using the pGEM-cIgM-LCgenome as a template, PCR was carried out with a sense primer (TGTCTCGAGTGAAGGTCACCAAGGATGG) (SEQ ID NO: 11) and an antisense primer (TTAAGCTTGGAGAGGAGAGAGGGGAGAA) (SEQ ID NO: 12), so as to obtain Left Arm shown in SEQ ID NO: 13.

### (3) Center Arm

Based on the sequence analysis of the pGEM-cIgM-LCgenome, the sequence of an intron region located between the variable region and the constant region of chicken Igλ was determined, and Center Arm shown in SEQ ID NO: 14 (wherein a blasticidin resistance gene having loxP sequences at both ends was inserted into the midstream thereof) was then synthesized.

### (4) Right Arm

The sequence of a region downstream of the constant region of chicken Igλ was determined using the pGEM-cIgM-LCgenome, and Right Arm shown in SEQ ID NO: 15 (wherein a neomycin resistance gene having lox2272 sequences at both ends was inserted into the midstream thereof) was then synthesized.

### (5) Construction of Targeting Vector

The above-described Left Arm, human Igλ variable region, Center Arm, human Igλ constant region, and Right Arm were incorporated into pBluescript to have the structure shown in FIG. 1, so as to obtain a human LC KI vector. The gene sequence of this vector is shown in SEQ ID NO: 16.

1-2. Knocking-in into Downstream of Chicken Antibody Light Chain (Igλ) Pseudogene Region (KI-C-IN Vector)

In order to insert a sequence for confirming gene conversion (a GC confirmation sequence; a human pseudogene consisting of a sequence derived from the variable region of human Igλ) (SEQ ID NO: 17) downstream of the pseudogene region of chicken Igλ, a targeting vector as shown in FIG. 2 was first produced. Methods for preparing individual parts will be described below.

### (1) Left Arm

The genomic DNA of DT40 was used as a template, and a 4.3-kb region (pseudogenes (pV) 1 to 3) located upstream of the promoter in the variable region was amplified by PCR using a sense primer (TTCTGTGAGCTGAGAAAAGGAGTGTA) (SEQ ID NO: 18) and an antisense primer (CCTGCATTGTGGCACAGCGGGGTT) (SEQ ID NO: 19), and it was then cloned into pCR4blunt-TOPO (Life Technologies) to obtain pCR4-cIgM-LC pV1 genome. Based on this sequence, a region comprising pV1-3 (SEQ ID NO: 20) was cloned, so as to obtain Left Arm.

### (2) Right Arm

Based on the sequence of the pCR4-cIgM-LCpV1 genome obtained in the above section, a portion that is a region between pV1 and the promoter (SEQ ID NO: 21) was cloned, so as to obtain Right Arm.

### (3) Construction of Targeting Vector

The above-described Left Arm, neomycin resistance gene, and Right Arm were incorporated into the pCR4blunt-TOPO to have the structure shown in FIG. 2, so as to obtain a KI-C-IN vector. The gene sequence of this vector is shown in SEQ ID NO: 22.

### 1-3. Insertion of GC Confirmation Sequence Downstream of Pseudogene Region of Chicken Antibody Light Chain (Igλ) (KI-C-IN-C Vector)

In order to insert a GC confirmation sequence downstream of the pseudogene region of chicken Igλ, a targeting vector as shown in FIG. 2 was produced. Methods for preparing individual parts will be described below.

### (1) Sequence for Confirmation of GC

Based on the variable region of human Igλ cloned in 1-1 above, a mutant sequence, in which 3 nucleotides were inserted into each CDR, and a mutant sequence, in which 3 nucleotides were deleted from each CDR, were designed, and these mutant sequences were then ligated to each other using the untranslated region sequence of a chicken Igλ pseudogene region to synthesize a sequence for confirmation of GC (SEQ ID NO: 17).

### (2) Construction of Targeting Vector

The sequence for confirmation of GC was incorporated into the KI-C-IN vector prepared in 1-2 above to have the structure shown in FIG. 2, so as to obtain a KI-C-IN-C vector. The gene sequence of this vector is shown in SEQ ID NO: 23.

### 1-4. Deletion of Pseudogene Region of Chicken Antibody Light Chain (Igλ) (KI-C-DE Vector)

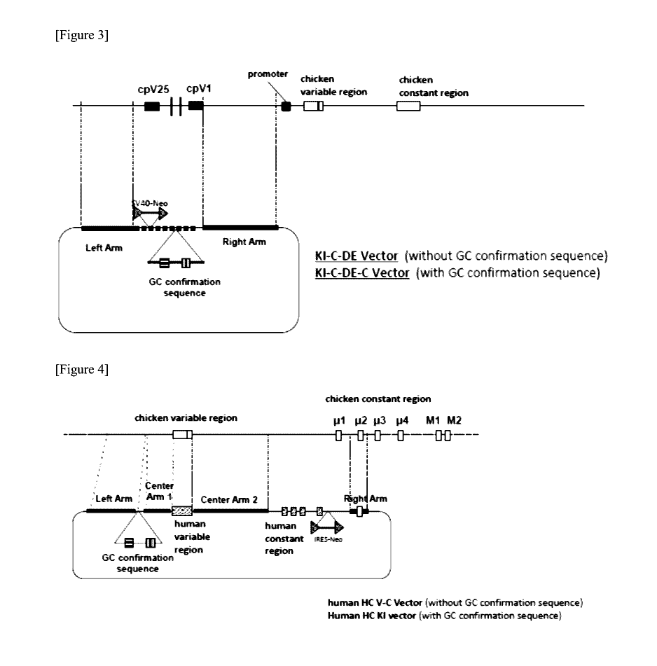

In order to delete the pseudogene region of chicken Igλ, a targeting vector as shown in FIG. 3 was produced. Methods for preparing individual parts will be described below.

### (1) Left Arm

The genomic DNA of DT40 was used as a template, and an approximately 3.0-kb region located upstream of pV25 was amplified by PCR using a sense primer (CGCTTTGTACGAACGTTGTCACGT) (SEQ ID NO: 24) and an antisense primer (TACCTGAAGGTCTCTTTGTGTTTTG) (SEQ ID NO: 25), and it was then cloned into the pCR4blunt-TOPO to obtain Left Arm shown in SEQ ID NO: 26.

### (2) Right Arm

The same Right Arm as that prepared in 1-2 above was used.

### (3) Construction of Targeting Vector

The above-described Left Arm, neomycin resistance gene, and Right Arm were incorporated into the pCR4blunt-TOPO to have the structure shown in FIG. 3, so as to obtain a KI-C-DE vector. The gene sequence of this vector is shown in SEQ ID NO: 27.

1-5. Replacement of Pseudogene Region of Chicken Antibody Light Chain (Igλ) with Sequence for Confirmation of GC (KI-C-DE-C Vector)

In order to replace the pseudogene region of chicken Igλ with a sequence for confirmation of GC, a targeting vector as shown in FIG. 3 was produced. Methods for preparing individual parts will be described below.

### (1) GC Confirmation Sequence

The GC confirmation sequence prepared in 1-2 above was used.

### (2) Construction of Targeting Vector

The above-described GC confirmation sequence was incorporated into the KI-C-DE vector produced in 1-3 above to have the structure shown in FIG. 3, so as to obtain a KI-C-DE-C vector. The gene sequence of this vector is shown in SEQ ID NO: 28.

1-6. Knocking-in into Constant Region and Variable Region of Chicken Antibody Heavy Chain (IgM)

In order to substitute a region ranging from the variable region of chicken IgM (IgM) to the constant region μ1 thereof with a human antibody heavy chain (IgG₁), a targeting vector as shown in FIG. 4 was produced. Methods for preparing individual parts will be described below.

### (1) Human IgG₁ Constant Region

Among sequences obtained by subjecting human genome chromosome 14 32.33 comprising human IgG₁ to shotgun sequencing, a sequence corresponding to IgG₁ was obtained (Accession No.: NW 001838121.1). The sequences of CH1 corresponding to the “C region” of “IGH1,” a hinge, CH2, CH3 and a transmembrane domain were extracted from a sequence between the nucleotide 41520 and the nucleotide 43117, and a sequence was then designed by replacing the intron with a mouse IgG₂ₐ-derived sequence. Thereafter, a human IgG₁ constant region sequence (SEQ ID NO: 29) was obtained by synthesis.

### (2) Human IgG₁ Variable Region

Based on the sequence of a human germ cell-derived IgG₁ variable region gene, a sequence was designed by ligating VH3-23, D5-12, and JH1 sequences to one another, and a chicken-derived secretory signal sequence or splicing signal sequence was then added to the sequence to prepare a human IgG₁ variable region (SEQ ID NO: 30).

### (3) Left Arm

The genomic DNA of DT40 was used as a template, and a region ranging from a region upstream of a chicken IgM variable region to the variable region was amplified by PCR using a sense primer (TTCCCGAAGCGAAAGCCGCGT) (SEQ ID NO: 31) and an antisense primer (ACTCACCGGAGGAGACGATGA) (SEQ ID NO: 32), and an approximately 4.1-kbp fragment was then cloned, so as to obtain pCR4 Blunt-TOPO-cIgM-HC pV1 genome. Based on this sequence, Left Arm (SEQ ID NO: 33), to the 3′ terminal side of which a Vlox sequence was added, was designed and synthesized.

### (4) Center Arm 1

Based on the approximately 4.1-kbp sequence ranging from the region upstream of the chicken IgM variable region to the variable region, which had been cloned upon the above-described cloning of the Left Arm, Center Arm 1 corresponding to an approximately 1.4-kbp region upstream of the chicken IgM variable region was synthesized (SEQ ID NO: 34).

### (5) Center Arm 2

The genomic DNA of DT40 was used as a template, and an approximately 4.3-kbp region located downstream of the chicken IgM variable region was amplified by PCR using a sense primer (GGGGATCCTGGGTCAGTCGAAGGGGGCG) (SEQ ID NO: 35) and an antisense primer (GTGCGGCCGCCAAAAGCGGTAAAATCCACCC) (SEQ ID NO: 36), and it was then cloned, so as to obtain Center Arm 2 shown in SEQ ID NO: 37.

### (6) Right Arm

The genomic DNA of DT40 was used as a template, and an approximately 0.9-kbp region comprising a chicken IgM constant region μ2 was amplified by PCR using a sense primer (CCAAACCACCTCCTGGTGTCC) (SEQ ID NO: 38) and an antisense primer (CAAACCAAAACTACGGATTCTCTGACC) (SEQ ID NO: 39), and it was then cloned, so as to obtain Right Arm shown in SEQ ID NO: 40.

### (7) Construction of Targeting Vector

The above-described Left Arm, multi-cloning site, Center Arm1, human IgG₁ variable region, blasticidin resistance gene, Center Arm 2, human IgG₁ constant region, neomycin resistance gene, and Right Arm were incorporated into pBluescript to have the structure shown in FIG. 4, so as to obtain a human HC V-C vector. The gene sequence of this vector is shown in SEQ ID NO: 41.

1-7. Knocking-in into Constant Region, Variable Region, and Pseudogene Region of Chicken Antibody Heavy Chain (IgM) (Human HC KI Vector)

In order to insert a sequence for confirming gene conversion (a GC confirmation sequence; a human pseudogene consisting of a human Igγ variable region-derived sequence), a human IgG₁ variable region, and a human IgG₁ constant region into a region ranging from a region downstream of the pseudogene region of chicken IgM to a constant region μ1 thereof, a targeting vector as shown in FIG. 4 was produced. Methods for preparing individual parts will be described below.

### (1) Human IgG₁ Constant Region

The human IgG₁ constant region sequence described in 1-6 above was used.

### (2) Human IgG₁ Variable Region

The human IgG₁ variable region described in 1-6 above was used.

### (3) Sequence for Confirming Gene Conversion

Based on the variable region of human Igγ designed in 1-6 above, a mutant sequence was designed by deleting one nucleotide from CDR1, another mutant sequence was designed by deleting two nucleotides from CDR2, and another mutant sequence was designed by inserting one nucleotide into CDR3. These mutant sequences were ligated to one another using the untranslated region sequence of a chicken Igγ pseudogene region to synthesize a GC confirmation sequence (SEQ ID NO: 42).

### (4) Left Arm

The Left Arm described in 1-6 above was used.

### (5) Center Arm 1

The Center Arm 1 described in 1-6 above was used.

### (6) Center Arm 2

The Center Arm 2 described in 1-6 above was used.

### (7) Right Arm

The Right Arm described in 1-6 above was used.

### (8) Construction of Targeting Vector

The above-described Left Arm, GC confirmation sequence, Center Arm 1, human IgG₁ variable region, blasticidin resistance gene, Center Arm 2, human IgG₁ constant region, neomycin resistance gene, and Right Arm were incorporated into pBluescript to have the structure shown in FIG. 4, so as to obtain a human HC KI vector. The gene sequence of this vector is shown in SEQ ID NO: 43.

### 1-8. Construction of Vector for Expressing Cre Recombinase

Since a drug resistance gene has been incorporated into the above-produced vector in such a form that loxP sequences have been added to both ends of the gene, Cre recombinase is allowed to act on the vector after completion of the gene transfer, so that this gene can be removed. Hence, a Cre recombinase gene shown in SEQ ID NO: 44 was incorporated into pEGFP-C1 (BD Bioscience, #6084-1) to produce Cre EGFP-C1 shown in SEQ ID NO: 45.

2. Production of Cell Lines into Light Chain of which Human Sequence has been Inserted or Substituted

As a host, DT40, a cell line derived from chicken B cells, was used. The DT40 was cultured by the following method.

Using a CO₂ thermostatic chamber, the cells were cultured at 39.5° C. in the presence of 5% CO₂. IMDM (Life Technologies) was used as a medium, and 9% FBS, 1% chicken serum, 100 units/mL penicillin, 100 μg/mL streptomycin, and 50 μM monothioglycerol were added to the medium, and the thus obtained medium was then used. Hereinafter, unless otherwise specified, the “medium” indicates a medium having the aforementioned composition.

In order to evaluate gene conversion in a light chain, five types of cell lines shown in the following Table 1 were prepared.

TABLE 1

| | Light chain | | | |
|---|---|---|---|---|
| | Pseudogene region | | | |
| | | Sequence for | | |
| | Chicken- | verification | | |
| | derived | of genetic | Variable | Constant |
| Cell line | sequence | recombination | region | region |
| Wild type | Yes | No | Chicken | Chicken |
| LP1 | Yes | No | Human | Human |
| LP9 | Yes | Yes | Human | Human |
| LP18 | No | No | Human | Human |
| LP20 | No | Yes | Human | Human |

Methods for producing these cell lines will be described below.

2-1. Production of Cell Lines in which Variable Region and Constant Region have been Replaced with Human-Type Regions

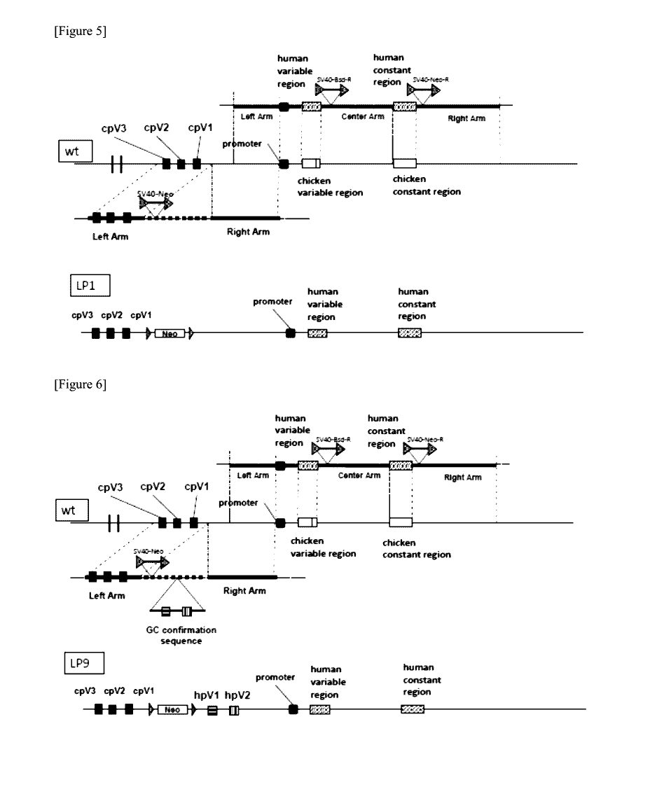

The human LC KI vector produced in 1-1 above was linearized with the restriction enzyme NotI, and it was then introduced into DT40 cells by electroporation. The resulting cells were cultured in a medium, to which 2 mg/mL G418 and 10 μg/mL blasticidin had been added, for 7 to 10 days. Thereafter, the growing cells were subjected to genotyping by PCR, and a cell line in which gene substitution had occurred in a gene locus of interest was selected. The Cre_pEGFP-C1 produced in 1-8 above was introduced into the selected cell line, and the resulting cell line was then subjected to single cell sorting by flow cytometry, so as to select GFP-positive cells. Thereafter, the GFP-positive cells were subjected to genotyping by PCR, so as to obtain a cell line 37-3, in which the removal of the drug resistance genes could be confirmed. Thereafter, the KI-C-IN vector produced in 1-2 above was linearized with the restriction enzyme NotI, and it was then introduced into the cell line 37-3. The cells were cultured in a medium, to which 2 mg/mL G418 had been added, for 7 to 10 days. Thereafter, the growing cells were subjected to genotyping by PCR, so as to obtain a cell line LP1, in which gene insertion had occurred in a gene locus of interest (FIG. 5).

2-2. Production of Cell Line in which Variable Region and Constant Region have been Replaced with Human-Type Regions, and Further, in which Sequence for Confirming Gene Conversion (GC Sequence) has been Inserted Downstream of Chicken Pseudogene Region

The KI-C-IN-C vector produced in 1-3 above was linearized with the restriction enzyme NotI, and it was then introduced into the cell line 37-3 obtained in the above section. The resulting cells were cultured in a medium, to which 2 mg/mL G418 had been added, for 7 to 10 days. Thereafter, the growing cells were subjected to genotyping by PCR, so as to obtain a cell line LP9, in which gene insertion had occurred in a gene locus of interest (FIG. 6).

2-3. Production of Cell Line in which Variable Region and Constant Region have been Replaced with Human-Type Regions, and Further, in which Chicken Pseudogene Region has been Deleted

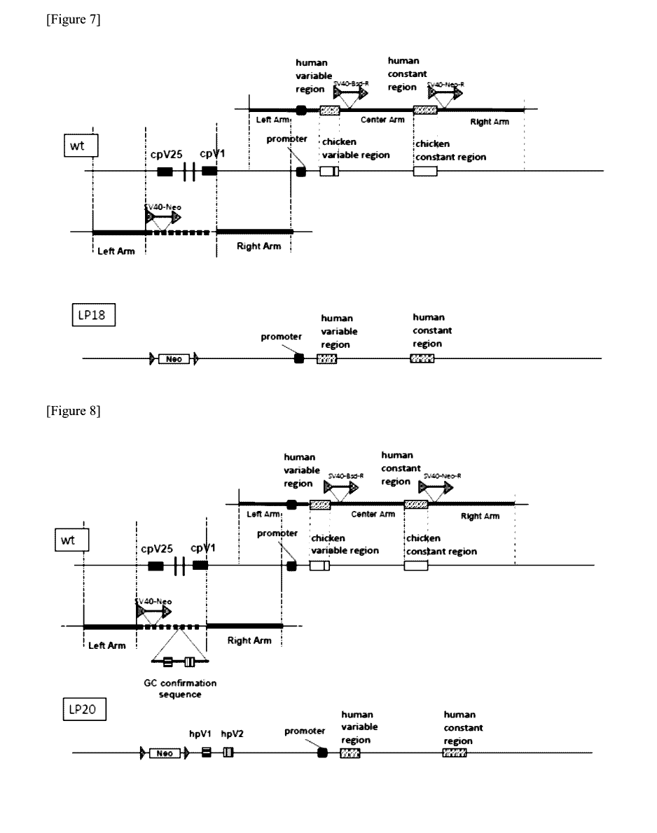

The KI-C-DE vector produced in 1-4 above was linearized with the restriction enzyme NotI, and it was then introduced into the cell line 37-3 obtained in 2-1 above. The resulting cells were cultured in a medium, to which 2 mg/mL G418 had been added, for 7 to 10 days. Thereafter, the growing cells were subjected to genotyping by PCR, so as to obtain a cell line LP18, in which gene insertion had occurred in a gene locus of interest (FIG. 7).

2-4. Production of Cell Line in which Variable Region and Constant Region have been Replaced with Human-Type Regions, and Further, in which Chicken Pseudogene Region has been Replaced with Sequence for Confirming Gene Conversion (GC Sequence)

The KI-C-DE-C vector produced in 1-5 above was linearized with the restriction enzyme NotI, and it was then introduced into the cell line 37-3 obtained in 2-1 above. The resulting cells were cultured in a medium, to which 2 mg/mL G418 had been added, for 7 to 10 days. Thereafter, the growing cells were subjected to genotyping by PCR, so as to obtain a cell line LP20, in which gene substitution had occurred in a gene locus of interest (FIG. 8).

### 3. Confirmation of Antibody Expression (Flow Cytometry and ELISA)

### 3-1. Analysis by Flow Cytometry

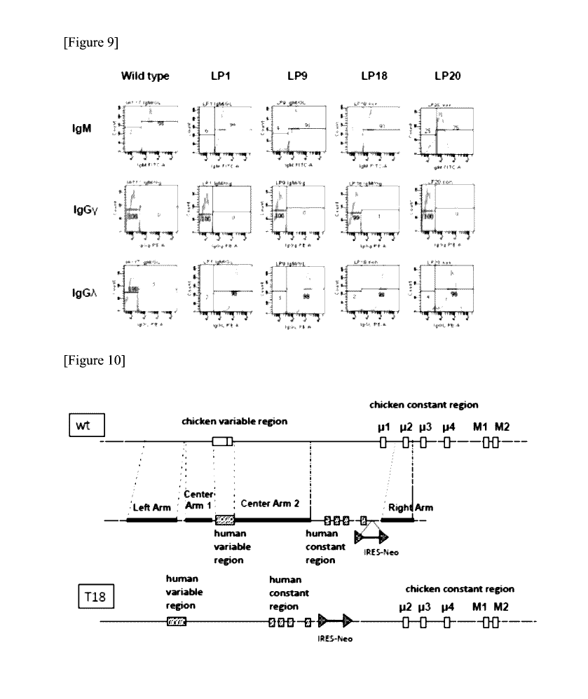

An antibody expressing on the surface of cells of each of the cell lines produced in 2 above was analyzed. Each cell line was cultured, and the cultured cells were then dispensed on a 96-well plate (Nunc, 249662) in an amount of 5×10⁵ cells/well. The cells were washed with 150 μL of FACS buffer (PBS containing 0.3% bovine serum albumin (BSA)) twice, and 50 μL of a primary antibody solution, which had been prepared by diluting Goat anti-Chicken IgM-FITC conjugate (Bethyl, A30-102F-20), Goat Anti-Human IgG (Gamma chain specific) R-PE conjugate (Southern biotech, A2040-09) or Goat Anti-human lambda PE conjugate (Southern biotech, 2070-09) with FACS buffer, 1000 times, 500 times, and 500 times, respectively, was added to the resulting cells. The mixture was incubated on ice in a dark place for 30 minutes, and it was then washed with 150 μL of FACS buffer twice. Thereafter, 200 μL of a PI solution, which had been 1000 times diluted with FACS buffer, was added to the reaction mixture, and was then suspended therein. This suspension was subjected to a flow cytometric analysis, and the expression of chicken IgM and human Igγ (heavy chain) or human Igλ (light chain) was analyzed. As a result, it was confirmed that, as shown in FIG. 9, only chicken IgM was expressed in wild-type DT40, whereas chicken IgM and human Igγ were expressed in the transformed cell lines LP1, LP9, LP18, and LP20. In all of the cell lines, the expression of Igγ that had not undergone gene transfer was not observed.

### 3-2. Analysis by ELISA

The concentrations of chicken IgM and human IgG₁, which were secreted from the cells produced in 2 above into culture supernatants, were measured. Individual cell lines were suspended in a medium containing no chicken serum to a concentration of 4×10⁵ cells/mL, and 2 mL each of the suspension was then plated on each well of a 24-well plate, followed by performing a culture for 3 days. Thereafter, the culture solution was recovered, was then filtrated through a 0.22-μm filter, and was then used in the measurement.

The method of measuring IgG is as follows. 20 μL of 1.0 μg/mL Goat anti-Human IgG-Fc affinity purified (Bethyl, A80-104A) was dispensed into an immuno 384-well plate Maxisorp (Nunc, 464718), and it was then reacted at room temperature for 1 hour or more, so that it was immobilized on the plate. Thereafter, the resultant on the plate was washed with a washing solution (PBS containing 0.05% Tween 20) five times, 50 μL of a blocking solution (PBS containing 1% BSA) was then added thereto, and the mixture was then reacted at room temperature for 30 minutes. Thereafter, the reaction mixture was washed with a washing solution five times, 20 μL of a measurement sample or Human IgG1 Lambda-UNLB (Southern Biotech, 0151L-01) serving as a standard substance was then added thereto, and the obtained mixture was then reacted at room temperature for 1 hour. Thereafter, the reaction mixture was washed with a washing solution five times, 20 μL of Goat anti-Human IgG-Fc HRP conjugated (Bethyl, A80-104P), which had been 1000 times diluted with PBS containing 1% BSA and 0.05% Tween 20, was then added thereto, and the obtained mixture was then reacted at room temperature for 1 hour. Thereafter, the reaction mixture was washed with a washing solution five times, 20 μL of TMB+(Dako, S159985) was then added thereto, and the obtained mixture was subjected to a coloring reaction at room temperature for 3 minutes. Subsequently, 20 μL of 1 N sulfuric acid was added to the reaction mixture to termination the reaction. Using Infinite M1000 (TECAN), the absorbance at 450 nm was measured.

For the measurement of IgM, the system of AlphaLISA Immunoassay (Perkin Elmer) was used. Goat anti-chicken IgM antibody (Bethyl, A30-102A) was labeled with biotin, employing Chromalink™ Biotin Labeling Reagent (SoluLink Inc., #B1001-105), so that a biotinylated antibody was prepared. Subsequently, Goat anti-chicken IgM antibody (Bethyl, A30-102A) was allowed to bind to Unconjugated AlphaLISA Acceptor Beads (Perkin Elmer), so that antibody-bound Alpha Screen beads were prepared. These biotin-labeled antibody and antibody-bound Alpha Screen beads were each diluted with an assay buffer (PBS containing 1% BSA) to concentrations of 4.5 nM and 50 μg/mL, respectively. Thereafter, they were mixed with each other in equal amounts, so as to prepare a chicken IgM concentration measurement solution. 20 μL of the chicken IgM concentration measurement solution was mixed with 5 μL of a measurement sample or the chicken serum (GIBCO, 16110) serving as a standard substance on a 96-well plate (Nunc), and the mixed solution was then dispensed into Alpha Plate-384 (Perkin Elmer) in an amount of 12.5 μL each. The mixed solution was reacted at room temperature for 60 minutes, and thereafter, 12.5 μL of AlphaScreen Streptavidin Donor Beads (Perkin Elmer), which had been adjusted to a concentration of 80 μg/mL with an assay buffer, was added to the reaction solution, and the obtained mixture was then reacted at room temperature under light shielding conditions for 30 minutes. Thereafter, using EnSpire (Perkin Elmer), an analysis was carried out.

The measurement results are shown in Table 2. It was confirmed that, as with the wild-type DT40, chicken IgM was secreted also in the transformed cell lines LP1, LP9, LP18 and LP20.

TABLE 2

| Cell line | IgG (μg/mL) | IgM (μg/mL) |
|---|---|---|
| Wild type | <0.01 | 2.30 |
| LP1 | <0.01 | 0.09 |
| LP9 | <0.01 | 0.10 |
| LP18 | <0.01 | 0.09 |
| LP20 | <0.01 | 0.03 |

### 4. Confirmation of Gene Conversion

The cell lines, in which the expression of human Igλ (light chain) had been confirmed in 3 above, were cultured in a medium containing 2.5 ng/mL TSA for 20 days, and genomic DNA was then extracted from 1×10⁶ cells. This genomic DNA was used as a template, and a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47). Thereafter, the resultant was purified by agarose gel extraction, and was then concentrated using QIAGEN PCR Purification Kit (QIAGEN). Using the Next-Generation Sequencing Analysis System of Roche Diagnostics, the sequence of this sample was analyzed, and the presence or absence of gene conversion was evaluated based on whether or not a gene sequence identical to the inserted human pseudogene sequence would be present in the variable region. The results are shown in Table 3. It was confirmed that gene conversion had not occurred in LP1 and LP18 that did not comprise the human pseudogene sequence, whereas such gene conversion had occurred in LP9 and LP20 having the human pseudogene sequence.

TABLE 3

| | | Number of sequences | |
|---|---|---|---|
| | | with genetic | |
| Cell line | Number of sequences | recombination | Frequency |
| LP1 | 785 | 0 | 0.0% |
| LP9 | 344 | 18 | 5.2% |
| LP18 | 378 | 0 | 0.0% |
| LP20 | 634 | 31 | 4.9% |

5. Production of Cell Line, in which Human Sequence has been Inserted and/or Substituted into Heavy Chain

In order to evaluate gene conversion in a heavy chain, the three types of cell lines shown in Table 4 were prepared.

TABLE 4

| | Heavy chain | | | |
|---|---|---|---|---|
| | Pseudogene region | | | |
| | | Sequence for | | |
| | Chicken- | verification | | |
| | derived | of genetic | Variable | Constant |
| Cell line | sequence | recombination | region | region |
| Wild type | Yes | No | Chicken | Chicken |
| T18 | Yes | No | Human | Human |
| T11, T12 | Yes | Yes | Human | Human |

Methods for producing these cell lines will be described below.

5-1. Production of Cell Line in which Variable Region and Constant Region of Heavy Chain have been Replaced with Human-Type Regions

The human HC V-C vector produced in 1-6 above was linearized with the restriction enzyme Sail, and it was then introduced into the cell line 37-3 obtained in 2-2 above. The resulting cells were cultured in a medium, to which 2 mg/mL G418 and 10 μg/mL blasticidin had been added, for 7 to 10 days. Thereafter, the growing cells were subjected to genotyping by PCR, and a cell line T18 in which gene substitution had occurred in a gene locus of interest was obtained (FIG. 10).

5-2. Production of Cell Line in which Variable Region and Constant Region of Heavy Chain have been Replaced with Human-Type Regions, and Sequence for Confirming Gene Conversion (GC Confirmation Sequence) has been Inserted Downstream of Pseudogene Region

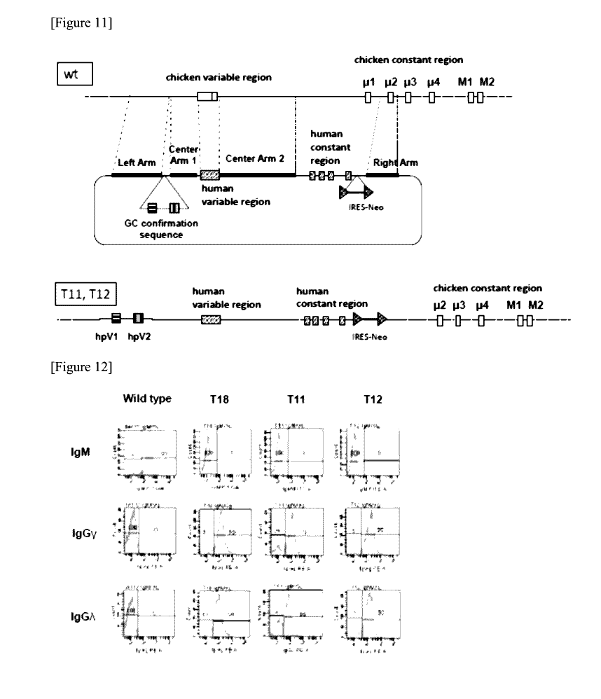

The human HC KI vector produced in 1-7 above was linearized with the restriction enzyme Sail, and it was then introduced into the cell line 37-3 obtained in 2-2 above. The resulting cells were cultured in a medium, to which 2 mg/mL G418 and 10 μg/mL blasticidin had been added, for 7 to 10 days. Thereafter, the growing cells were subjected to genotyping by PCR, and cell lines T11 and T12, in which gene substitution had occurred in a gene locus of interest was obtained (FIG. 11).

### 6. Confirmation of Antibody Expression (Flow Cytometry and ELISA)

### 6-1. Analysis by Flow Cytometry

Based on the method described in 3-1 above, the expression of an antibody in the cells produced in 5 above was confirmed. The results are shown in FIG. 12. It was confirmed that only chicken IgM was expressed in wild-type DT40, whereas the expression of chicken IgM disappeared and instead, human Igγ and Igλ were expressed in the transformed cell lines T18, T11 and T12.

### 6-2. Analysis by ELISA

Based on the method described in 3-2 above, the antibodies secreted from the cells produced in 5 above into a culture supernatant were measured. The results are shown in Table 5. As with 6-1 above, it was confirmed that only chicken IgM was secreted from wild-type DT40, whereas human IgG₁ was secreted from T18, T11 and T12, into which human IgG₁ had been introduced.

TABLE 5

| Cell line | IgG (μg/mL) | IgM (μg/mL) |
|---|---|---|
| Wild type | <0.01 | 2.30 |
| T18 | 1.00 | 0.03 |
| T11 | 0.40 | <0.01 |
| T12 | 0.69 | <0.01 |

### 7. Confirmation of Gene Conversion

Using the cell lines in which the expression of an antibody had been confirmed in 6 above, gene conversion was confirmed. The genomic DNA extracted by the method described in 4 above was used as a template, and a heavy chain variable region was amplified by PCR using a sense primer (CTATGCGCCTTGCCAGCCCGCTCAGTCCGTCAGCGCTCTCT) (SEQ ID NO: 48) and an antisense primer to which an identification tag had been added (CGTATCGCCTCCCTCGCGCC ATCAGNNNNNNNNNNCGGTGACCAGGGTGCCCTG) (SEQ ID NO: 49). Thereafter, a sequence analysis was carried out based on the method described in 4 above. The results are shown in Table 6. It was confirmed that gene conversion had not occurred at all in T18 containing no human pseudogenes, whereas such gene conversion had occurred in T11 and T12 having such a human pseudogene sequence.

TABLE 6

| | | Number of sequences | |
|---|---|---|---|
| | | with genetic | |
| Cell line | Number of sequences | recombination | Frequency |
| T18 | 1125 | 0 | 0.0% |
| T11 | 3038 | 415 | 13.4% |
| T12 | 4108 | 305 | 7.4% |

Production of L5/H5 Cell Line, in which Pseudogene Region Consisting of Five Human Variable Region-Derived Sequences has been Inserted into Each of Light Chain and Heavy Chain

### 8. Production of Vectors

8-1. Knocking-in into Chicken Antibody (Igλ) Pseudogene Region

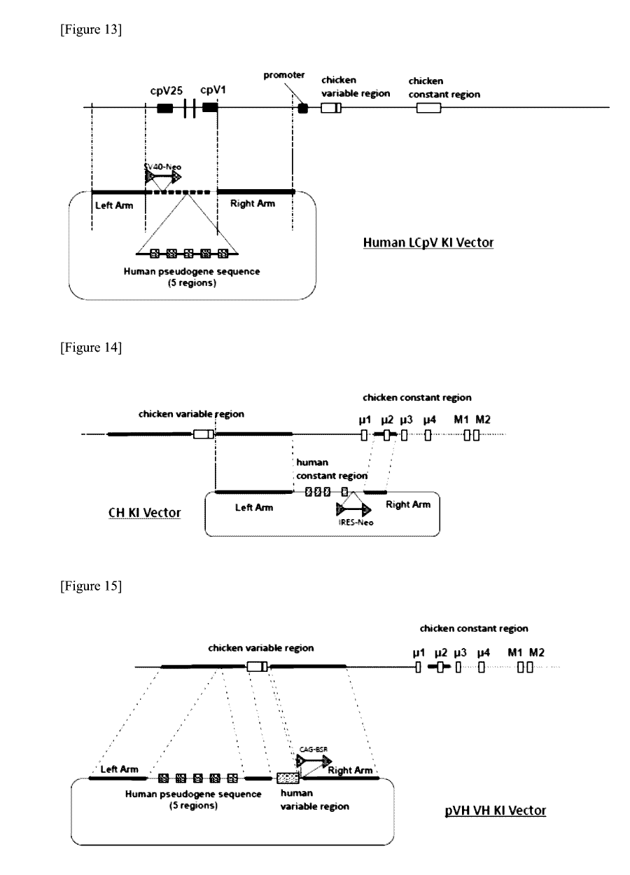

In order to substitute the pseudogene region of chicken Igλ with a pseudogene consisting of a sequence derived from the variable region of human Igλ (a human light chain pseudogene), a targeting vector as shown in FIG. 13 was produced. The production method will be described below.

(1) Designing and Production of Human Antibody Light Chain Pseudogene Sequence (pVL5)

Based on the gene sequences of immunoglobulin λ light chain variable regions, which had not caused V(D)J recombination in the human genome in the database, five sequences were selected from each of CDR1, CDR2 and CDR3, and the framework sequence of the Igλ variable region cloned in 1-1 above was then combined therewith, so as to design five sequences, namely, pVL001 (SEQ ID NO: 69), pVL002 (SEQ ID NO: 70), pVL003 (SEQ ID NO: 71), pVL004 (SEQ ID NO: 72) and pVL005 (SEQ ID NO: 73). Thereafter, a human light chain pseudogene sequence pVL5 (SEQ ID NO: 50) comprising these sequences was designed, and was then subjected to gene synthesis.

### (2) Production of Targeting Vector

On the basis of the KI-C-DE vector produced in 1-4 above, the GC confirmation sequence was replaced with the human antibody light chain pseudogene sequence pVL5 described in the above section to produce a human LCpV KI vector. The gene sequence of this vector is shown in SEQ ID NO: 51.

### 8-2. Substitution of Chicken Antibody Heavy Chain (IgM) Constant Region

In order to substitute the heavy chain constant region of chicken IgM with human IgG₁, a targeting vector as shown in FIG. 14 was produced. The production method will be described below.

### (1) Human IgG₁ Constant Region

The human IgG₁ constant region produced in 1-6 above was used.

### (2) Left Arm

The Center Arm 2 produced in 1-6 above was used.

### (3) Right Arm

The Right Arm produced in 1-6 above was used.

### (4) Production of Targeting Vector

The above-described Left Arm, human IgG₁ constant region sequence, neomycin resistance gene, and Right Arm were incorporated into pBluescript to produce a human CH KI vector. The gene sequence of this vector is shown in SEQ ID NO: 52.

8-3. Insertion into Chicken IgM Variable and Pseudogene Regions

### (1) Human IgG₁ Variable Region

The human IgG₁ variable region produced in 1-6 above was used.

(2) Designing and Production of Human Antibody Heavy Chain Pseudogene Sequence (pVH5)

Based on the sequences of human immunoglobulin heavy chain variable regions in the database, five sequences were selected from each of CDR1, CDR2 and CDR3, and the framework sequence of the variable region cloned in 1-6 above was then combined therewith, so as to design five sequences, namely, pVH001 (SEQ ID NO: 89), pVH002 (SEQ ID NO: 90), pVH003 (SEQ ID NO: 91), pVH004 (SEQ ID NO: 92) and pVH005 (SEQ ID NO: 93). Thereafter, a human antibody heavy chain pseudogene sequence pVH5 (SEQ ID NO: 53) comprising these sequences was designed, and was then subjected to gene synthesis.

### (3) Left Arm

The Left Arm produced in 1-6 above was used.

### (4) Center Arm

The Center Arm 1 produced in 1-6 above was used.

### (5) Right Arm

The Center Arm 2 produced in 1-6 above was used. The confirmed sequence of the Right Arm is shown in SEQ ID NO: 54.

### (6) Production of Targeting Vector

The above-described Left Arm, human antibody heavy chain pseudogene sequence pVH5, Center Arm, human IgG₁ variable region sequence, blasticidin resistance gene, and Right Arm were incorporated into pBluescript to obtain a human pVH VH KI vector. The gene sequence of this vector is shown in SEQ ID NO: 55.

9. Production of L5/H5 Cell Line, in which Five Human Pseudogenes have been Introduced into Each of Gene Loci of Chicken Antibody Light Chain and Heavy Chain

The human CH KI vector produced in 8-2 above, the human pVH VH KI vector produced in 8-3 above, and the human LC pV KI vector produced in 8-1 above were introduced into the cell line 37-3 obtained in 2-2 above, so as to produce a L5/H5 cell line (FIG. 15).

The human CH KI vector, which had been linearized with the restriction enzyme SalI, was introduced into the cell line 37-3, and the cells were then cultured in a medium containing 2 mg/mL G418. The growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

Subsequently, the human pVH VH KI vector, which had been linearized with the restriction enzyme SalI, was introduced into the cell line 37-3, and the cells were then cultured in a medium containing 10 μg/mL blasticidin. The growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

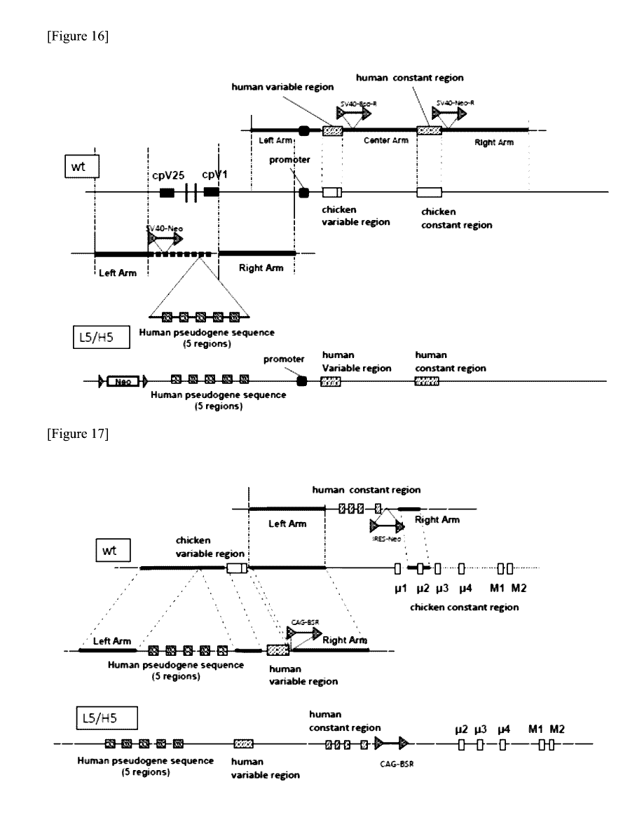

Finally, the human LC pV KI vector, which had been linearized with the restriction enzyme NotI, was introduced into the cell line 37-3, and the cells were then cultured in a medium containing 2 mg/mL G418. The growing cells were subjected to genotyping by PCR, so as to obtain cell lines B-B10 and J-D3, in each of which substitution had occurred in a gene locus of interest (FIG. 16 and FIG. 17).

### 10. Confirmation of Antibody Expression (Flow Cytometry and ELISA)

### 10-1. Analysis by Flow Cytometry

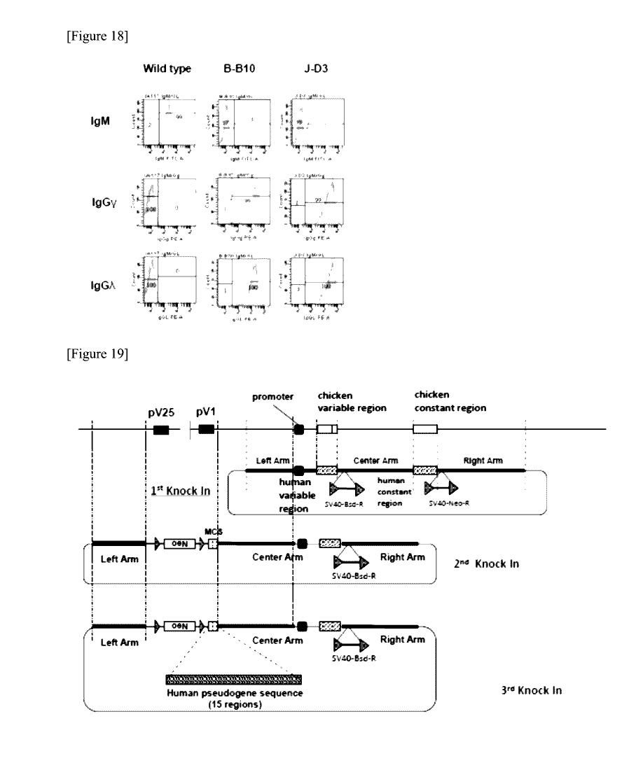

Based on the method described in 3-1 above, the expression of an antibody in the cells produced in 9 above was confirmed. The results are shown in FIG. 18. It was confirmed that only chicken IgM was expressed in wild-type DT40, whereas the expression of chicken IgM disappeared and instead, human Igγ and Igλ were expressed in the transformed cell lines B-B10 and J-D3.

### 10-2. Analysis of ELISA

Based on the method described in 3-2 above, the antibodies secreted from the cells produced in 9 above were measured. The results are shown in Table 7. As with 10-1 above, it was confirmed that only chicken IgM was secreted from wild-type DT40, whereas human IgG₁ was secreted from the transformed cell lines B-B10 and J-D3.

TABLE 7

| Cell line | IgG (μg/mL) | IgM (μg/mL) |
|---|---|---|
| Wild type | <0.01 | 2.55 |
| B-B10 | 2.19 | 0.01 |
| J-D3 | 3.45 | <0.01 |

### 11. Confirmation of Gene Conversion

Using the cell lines in which the expression of an antibody had been confirmed in 10 above, gene conversion was confirmed. The genomic DNA extracted from the cells, which had been cultured in the presence of TSA for 42 days, according to the method described in 4 above was used as a template, and a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56) and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out according to the method described in 4 above. The results obtained by analyzing the sequences of the light chain and heavy chain variable regions in the cell line B-B10 are shown in Table 8 and Table 9, respectively. A clone comprising the sequence derived from the inserted human pseudogene was confirmed in both of the light chain and heavy chain variable regions, and thus, it was demonstrated that a variety of sequences could be generated as a result of gene conversion. Moreover, it was also confirmed that a variety of sequences could be generated as a result of gene conversion even in the cell line J-D3.

TABLE 8

| # | % | CDR1 | CDR2 | CDR3 |
|---|---|---|---|---|
| 1 | 0.183 | VL | pVL003 | VL |
| 2 | 0.046 | Not matched | VL | pVL003 |
| 3 | 0.046 | VL | VL | pVL004 |

VL: sequence inserted into variable region
pVL001-005: sequences inserted into pseudogene region
Not matched: sequence that is not completely matched with either VL or pVL (which is probably caused by partial genetic recombination or mutation)

TABLE 9

| # | % | CDR1 | CDR2 | CDR3 |
|---|---|---|---|---|
| 1 | 11.176 | pVH004 | VH | VH |
| 2 | 0.131 | pVH001 | VH | VH |
| 3 | 0.131 | pVH004 | Not matched | VH |
| 4 | 0.065 | pVH003 | VH | VH |

VH: sequence inserted into variable region
pVH001-005: sequences inserted into pseudogene region
Not matched: sequence that is not completely matched with either VL or pVL (which is probably caused by partial genetic recombination or mutation)

Production of L15/H15 Cell Line, in which 15 Human Variable Region-Derived Sequences have been Inserted into Each of Chicken Antibody Light Chain and Heavy Chain Gene Loci

### 12. Production of Vectors

12-1. Knocking-in into Chicken Antibody Light Chain (Igλ) Variable and Constant Regions

Based on the method described in 1-1 above, a targeting vector, Lambda-VL-CL KI vector 2.0, shown in FIG. 19 was produced.

(1) Variable region and constant region of human Igλ

The variable region and the constant region produced in 1-1 above were used.

### (2) Left Arm

The Left Arm produced in 1-1 above was used.

### (3) Center Arm

The Center Arm produced in 1-1 above was used.

### (4) Right Arm

Based on the sequence of a region downstream of the constant region of a chicken antibody light chain, which was determined in 1-1 above, Right Arm was synthesized.

### (5) Construction of Targeting Vector

The above-described Left Arm, human Igλ variable region, Center Arm, human Igλ constant region, and Right Arm were incorporated into pBluescript to have the structure shown in FIG. 19, so as to obtain Lambda-VL-CL KI vector 2.0 (SEQ ID NO: 58).

### 12-2. Knock-Out of Chicken Antibody Light Chain (Igλ) Pseudogene

### (1) Human Igλ Variable Region

The variable region sequence described in 1-1 above was used.

### (2) Left Arm

The Left Arm described in 1-2 above was used.

### (3) Center Arm

The Left Arm described in 1-1 above was used as Center Arm.

### (4) Right Arm

The Center Arm described in 1-1 above was used as Right Arm.

### (5) Multi-Cloning Site

For insertion of a human antibody light chain pseudogene sequence pVL15, a multi-cloning site shown in SEQ ID NO: 59 was designed.

### (6) Construction of Targeting Vector

The above-described Left Arm, neomycin resistance gene, multi-cloning site, Center Arm, human Igλ variable region, blasticidin resistance gene, and Right Arm were incorporated into pUC19, so as to construct a targeting vector pVL KI_pUC19_step5_BrRev (SEQ ID NO: 60), as shown in FIG. 19.

12-3. Knocking-in into Chicken Antibody Light Chain (Igλ) Pseudogene and Variable Regions
(1) Human Antibody Light Chain Pseudogene Sequence (pVL15)

Based on the sequences of human immunoglobulin k variable regions in the database, 15 CDR1, CDR2 and CDR3 sequences were selected, and the framework sequence of the human Igλ variable region cloned in 1-1 above were then combined therewith, so as to design 15 sequences, namely, pVL101 (SEQ ID NO: 74), pVL102 (SEQ ID NO: 75), pVL103 (SEQ ID NO: 76), pVL104 (SEQ ID NO: 77), pVL105 (SEQ ID NO: 78), pVL106 (SEQ ID NO: 79), pVL107 (SEQ ID NO: 80), pVL108 (SEQ ID NO: 81), pVL109 (SEQ ID NO: 82), pVL110 (SEQ ID NO: 83), pVL111 (SEQ ID NO: 84), pVL112 (SEQ ID NO: 85), pVL113 (SEQ ID NO: 86), pVL114 (SEQ ID NO: 87), and pVL115 (SEQ ID NO: 88). A human antibody light chain pseudogene sequence pVL15 (SEQ ID NO: 61) comprising these sequences was designed, and was then subjected to gene synthesis.

### (2) Construction of Targeting Vector

The above-described human antibody light chain pseudogene sequence pVL15 was incorporated into the multi-cloning site of the pVL KI_pUC19 step6 BrRev produced in 12-2 above, so as to construct a targeting vector pVL KI_pUC19_step6_BrRev_pVL#odd (SEQ ID NO: 62) as shown in FIG. 19.

12-4. Knocking-in into Chicken Antibody Heavy Chain (IgM) Constant Region

The human CH KI vector produced in 8-2 above was used.

12-5. Knocking-in into Chicken Antibody Heavy Chain (IgM) Variable Region

### (1) Human IgG₁ Variable Region

The variable region sequence described in 1-6 above was used.

### (2) Left Arm

The Center Arm described in 1-6 above was used.

### (3) Right Arm

The Right Arm described in 1-6 above was used.

### (4) Construction of Targeting Vector

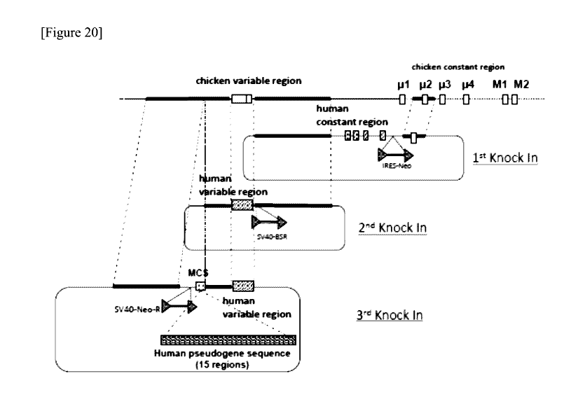

The above-described Left Arm, human IgG₁ variable region, blasticidin resistance gene, and Right Arm were incorporated into pUC19, so as to produce a targeting vector H(X)0•hV(B)_074-009/No4 (SEQ ID NO: 63) as shown in FIG. 20.

12-6. Knocking-in into Chicken Antibody Heavy Chain (IgM) Pseudogene Region
(1) Human Antibody Heavy Chain Pseudogene Sequence (pVH15)

Based on the sequences of human immunoglobulin heavy chain variable regions in the database, 15 CDR1, CDR2 and CDR3 sequences were selected, and the framework sequence of the human IgG₁ variable region cloned in 1-6 above was then combined therewith, so as to design 15 sequences, namely, pVH101 (SEQ ID NO: 94), pVH102 (SEQ ID NO: 95), pVH103 (SEQ ID NO: 96), pVH104 (SEQ ID NO: 97), pVH105 (SEQ ID NO: 98), pVH106 (SEQ ID NO: 99), pVH107 (SEQ ID NO: 100), pVH108 (SEQ ID NO: 101), pVH109 (SEQ ID NO: 102), pVH110 (SEQ ID NO: 103), pVH111 (SEQ ID NO: 104), pVH112 (SEQ ID NO: 105), pVH113 (SEQ ID NO: 106), pVH114 (SEQ ID NO: 107), and pVH115 (SEQ ID NO: 108). Thereafter, a human antibody heavy chain pseudogene sequence pVH15 (SEQ ID NO: 64) was designed by ligating these sequences to one another, and was then subjected to gene synthesis.

### (2) Left Arm

The Left Arm described in 1-6 above was used.

### (3) Right Arm

The Center Arm described in 1-6 above was used.

### (4) Multi-Cloning Site

For insertion of the human antibody heavy chain pseudogene sequence, a multi-cloning site shown in SEQ ID NO: 65 was designed.

### (5) Site-Specific Recombination Using Cre Recombinase (RMCE Method)

It has been known that if a pseudogene sequence region is prolonged, the transduction efficiency of homologous recombination is reduced. Thus, in order to easily insert a pseudogene sequence using Cre recombinase, an RMCE method was applied. First, a cassette sequence for RMCE comprising a loxm3 rev sequence and a loxm7 rev RE sequence was designed.

### (6) Construction of Targeting Vector

The Left Arm, neomycin resistance gene, cassette sequence for RMCE, multi-cloning site, Right Arm, and human IgG₁ variable region were incorporated into pUC19, and thereafter, the human antibody heavy chain pseudogene region pVH15 was incorporated into the multi-cloning site, so as to produce a targeting vector pVH KI_pUC19_step3p_VH#odd_15pVH_RMCE (SEQ ID NO: 66) as shown in FIG. 20.

### 13. Production of Cell Lines

The Lambda-VL-CL KI vector 2.0 prepared in 12-1 above, the pVL KI_pUC19_step5_BrRev prepared in 12-2 above, the pVL KI_pUC19_step6_BrRev_pVL#odd prepared in 12-3 above, the human CH KI vector prepared in 12-4 above, the H(X)0•hV(B)_074-009/No4 prepared in 12-5 above, and the pVH KI_pUC19_step3_pVH#odd_15pVH_RMCE prepared in 12-6 above were each introduced into DT40 cells, so as to produce cell lines.

First, the human CH KI vector, which had been linearized with the restriction enzyme SalI, was introduced into the cells, and the obtained mixture was then cultured in a medium containing 2 mg/mL G418. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

Subsequently, the Lambda-VL-CL KI vector 2.0, which had been linearized with the restriction enzyme NotI was introduced into DT40, and the obtained mixture was then culture in a medium containing 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

Thereafter, the pVL KI_pUC19_step5_BrRev, which had been linearized with the restriction enzyme NotI, was introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance genes had been removed.

Thereafter, the pVL KI_pUC19_step6_BrRev_pVL#odd, which had been linearized with the restriction enzyme NotI, was introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance genes had been removed.

Finally, the H(X)0•hV(B)_074-009/No4 linearized with the restriction enzyme ScaI and pVH KI_pUC19_step3_pVH#odd_15pVH_RMCE linearized with the restriction enzyme NotI were introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to obtain cell lines A12-3, A12-4, A12-5, B7-3, B7-9 and B7-11, from each of which the drug resistance genes had been removed.

### 14. Confirmation of Antibody Expression (Flow Cytometry and ELISA)

### 14-1. Analysis by Flow Cytometry

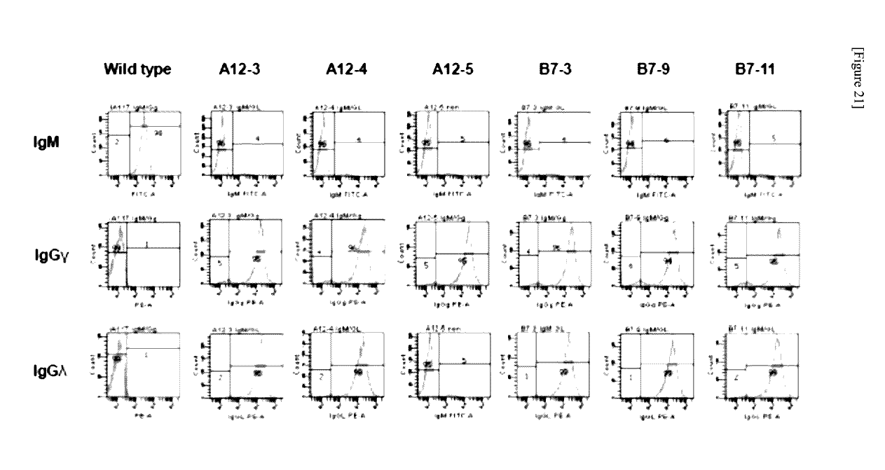

Based on the method described in 3 above, the expression of an antibody in the cells produced in 13 above was confirmed. The results are shown in FIG. 21 and FIG. 22. It was confirmed that only chicken IgM was expressed in wild-type DT40, whereas the expression of chicken IgM disappeared and instead, human Igγ and Igλ were expressed in the transformed cell lines A12-3, A12-4, A12-5, B7-3, B7-9 and B7-11 (FIGS. 21 and 22). It is to be noted that since the histogram of Igλ in A12-5 was incorrect in FIG. 21, FIG. 22 including correct data obtained before the priority date was attached herewith.

### 14-2. Analysis by ELISA

Based on the method described in 3-2 above, the antibodies secreted from the cells produced in 13 above were measured. The results are shown in Table 10. As with 10-1 above, it was confirmed that only chicken IgM was secreted from wild-type DT40, whereas human IgG₁ was secreted from the transformed cell lines A12-3, A12-4, A12-5, B7-3, B7-9 and B7-11.

TABLE 10

| Cell line | IgG (μg/mL) | IgM (μg/mL) |
|---|---|---|
| Wild type | <0.01 | 2.54 |
| A12-3 | 0.52 | <0.01 |
| A12-4 | 0.51 | <0.01 |
| A12-5 | 0.65 | 0.01 |
| B7-3 | 0.62 | 0.01 |
| B7-9 | 0.76 | 0.02 |
| B7-11 | 0.77 | 0.02 |

### 15. Confirmation of Gene Conversion

Using the cell lines in which the expression of an antibody had been confirmed in 10 above, gene conversion was confirmed. Using, as a template, the genomic DNA extracted from the cells, which had been cultured for 21 days or 42 days in the presence of TSA, by the method described in 4 above, a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56) and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line B7-11 cultured in the presence of TSA for 21 days are shown in Table 11 and Table 12, respectively. The results (the top 10) of the sequence analysis performed on the variable regions of the light chain and heavy chain in the cell line B7-11 cultured in the presence of TSA for 42 days are shown in Table 13 and Table 14, respectively. A clone comprising a sequence derived from a human pseudogene was found in the variable regions of both of the light chain and the heavy chain, and it was demonstrated that a variety of sequences were generated as a result of gene conversion. In addition, it was also confirmed that a variety of sequences were generated as a result of gene conversion even in other cell lines.

TABLE 11

| # | % | CDR1 | CDR2 | CDR3 |
|---|---|---|---|---|
| 1 | 0.327 | VL | pVL103 | VL |
| 2 | 0.327 | VL | pVL102 | VL |
| 3 | 0.196 | VL | VL | pVL114 |
| 4 | 0.196 | VL | pVL105 | VL |
| 5 | 0.131 | VL | pVL104 | VL |
| 6 | 0.131 | VL | VL | pVL112 |
| 7 | 0.131 | pVL105 | VL | VL |
| 8 | 0.065 | VL | Not matched | pVL106 |
| 9 | 0.065 | VL | pVL109 | VL |
| 10 | 0.065 | VL | VL | pVL110 |
| 11 | 0.065 | VL | pVL107 | VL |

VL: sequence inserted into variable region
pVL101-115: sequences inserted into pseudogene region
Not matched: sequence that is not completely matched with either VL or pVL (which is probably caused by partial genetic recombination or mutation)

TABLE 12

| # | % | CDR1 | CDR2 | CDR3 |
|---|---|---|---|---|
| 1 | 0.46 | pVH105 | VH | VH |
| 2 | 0.345 | pVH108 | VH | VH |
| 3 | 0.345 | VH | pVH107 | Not matched |
| 4 | 0.345 | pVH107 | VH | VH |
| 5 | 0.345 | pVH111 | pVH111 | VH |
| 6 | 0.46 | pVH112 | Not matched | VH |
| 7 | 0.23 | pVH109 | VH | VH |
| 8 | 0.23 | VH | pVH112 | VH |
| 9 | 0.23 | pVH105 | Not matched | VH |
| 10 | 0.115 | pVH108 | pVH108 | VH |
| 11 | 0.115 | VH | VH | pVH113 |
| 12 | 0.115 | VH | VH | pVH109 |
| 13 | 0.115 | pVH107 | VH | Not matched |
| 14 | 0.115 | pVH103 | Not matched | VH |
| 15 | 0.23 | pVH111 | Not matched | VH |
| 16 | 0.23 | pVH111 | Not matched | Not matched |
| 17 | 0.115 | pVH111 | VH | VH |

VL: sequence inserted into variable region
pVL101-115: sequences inserted into pseudogene region
Not matched: sequence that is not completely matched with either VL or pVL (which is probably caused by partial genetic recombination or mutation)

TABLE 13

| # | % | CDR1 | CDR2 | CDR3 |
|---|---|---|---|---|
| 1 | 0.175 | VL | pVL109 | VL |
| 2 | 0.175 | pVL105 | VL | VL |
| 3 | 0.175 | pVL105 | pVL105 | VL |
| 4 | 0.116* | VL | pVL105 | VL |
| 5 | 0.116* | VL | pVL105 | VL |
| 6 | 0.116 | Not matched | pVL105 | VL |
| 7 | 0.058 | VL | pVL109 | Not matched |
| 8 | 0.058 | VL | VL | pVL114 |
| 9 | 0.058 | VL | pVL102 | VL |
| 10 | 0.058 | VL | pVL108 | VL |

VL: sequence inserted into variable region
pVL: sequence inserted into pseudogene region
Not matched: sequence that is not completely matched with either VL or pVL (which is probably caused by partial genetic recombination or mutation)
*Sequences of portions other than CDRs are different, respectively.

TABLE 14

| # | % | CDR1 | CDR2 | CDR3 |
|---|---|---|---|---|
| 1 | 1.789 | pVH108 | VH | VH |
| 2 | 1.022 | pVH112 | VH | VH |
| 3 | 0.681 | pVH109 | VH | VH |
| 4 | 0.596 | VH | pVH112 | VH |
| 5 | 0.596 | pVH111 | Not matched | VH |
| 6 | 0.511 | pVH107 | VH | VH |
| 7 | 0.426 | pVH111 | pVH111 | VH |
| 8 | 0.341 | pVH111 | VH | VH |
| 9 | 0.256 | pVH108 | pVH108 | VH |
| 10 | 0.256 | VH | pVH107 | Not matched |

VH: sequence inserted into variable region
pVH: sequence inserted into pseudogene region
Not matched: sequence that is not completely matched with either VH or pVH (which is probably caused by partial genetic recombination or mutation)

Production of L15/H30 Cell Line, in which 15 Pseudogene Regions Each Consisting of Human Variable Region-Derived Sequence have been Inserted into Light Chain and 30 Such Pseudogene Regions have been Inserted into Heavy Chain

### 16. Production of Vectors

16-1. Knocking-in into Chicken Antibody Light Chain (Igλ) Variable and Constant Regions

The Lambda-VL-CL KI vector 2.0 produced in 12-1 above was used.

### 16-2. Knock-Out of Chicken Antibody Light Chain (Igλ) Pseudogene

The pVL KI_pUC19_step5_BrRev produced in 12-2 above was used.

16-3. Knocking-in into Chicken Antibody Light Chain (Igλ) Pseudogene and Variable Regions

The pVL KI_pUC19_step6_BrRev_pVL#odd produced in 12-3 above was used.

16-4. Knocking-in into Chicken Antibody Heavy Chain (IgM) Constant Region

The human CH KI vector produced in 8-2 above was used.

16-5. Knocking-in into Chicken Antibody Heavy Chain (IgM) Variable Region

### (1) Human IgG₁ Variable Region

The variable region sequence described in 1-6 above was used.

### (2) Left Arm

The Left Arm described in 1-6 above was used.

### (3) Center Arm

The Center Arm described in 1-6 above was used.

### (4) Right Arm

The Right Arm described in 1-6 above was used.

### (5) Cassette Sequence for RMCE

The cassette sequence for RMCE designed in 12-6 above was used.

### (6) Construction of Targeting Vector

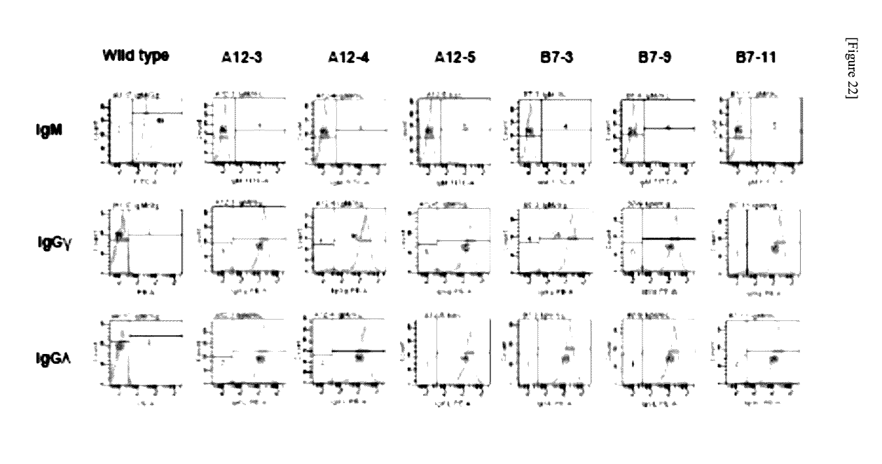

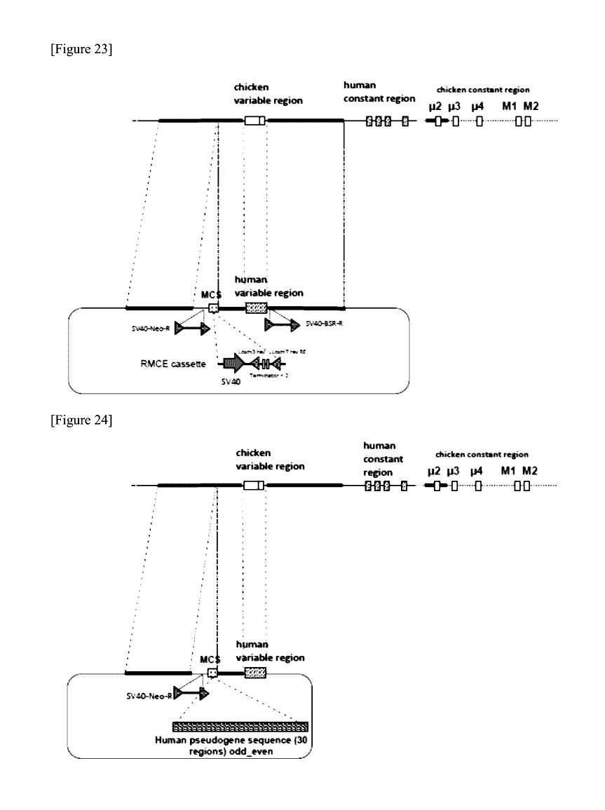

The above-described Left Arm, neomycin resistance gene, cassette sequence for RMCE, Center Arm, human IgG₁ variable region, and Right Arm were incorporated into pUC19, so as to produce a targeting vector pVH KI_pUC19_step5_BsrRev_RMCE_cassette (SEQ ID NO: 109) as shown in FIG. 23.

16-6. Knocking-in into Chicken Antibody Heavy Chain (IgM) Pseudogene Region
(1) Human Antibody Heavy Chain Pseudogene Sequence (pVH30)

Based on the sequences of human immunoglobulin heavy chain variable regions in the database, 30 CDR1, CDR2 and CDR3 sequences were selected, and the framework sequence of the human IgG₁ variable region cloned in 1-6 above was then combined therewith, so as to design 30 sequences, namely, pVH101 (SEQ ID NO: 94), pVH102 (SEQ ID NO: 95), pVH103 (SEQ ID NO: 96), pVH104 (SEQ ID NO: 97), pVH105 (SEQ ID NO: 98), pVH106 (SEQ ID NO: 99), pVH107 (SEQ ID NO: 100), pVH108 (SEQ ID NO: 101), pVH109 (SEQ ID NO: 102), pVH110 (SEQ ID NO: 103), pVH111 (SEQ ID NO: 104), pVH112 (SEQ ID NO: 105), pVH113 (SEQ ID NO: 106), pVH114 (SEQ ID NO: 107), pVH115 (SEQ ID NO: 108), pVH116 (SEQ ID NO: 110), pVH117 (SEQ ID NO: 111), pVH118 (SEQ ID NO: 112), pVH119 (SEQ ID NO: 113), pVH120 (SEQ ID NO: 114), pVH121 (SEQ ID NO: 115), pVH122 (SEQ ID NO: 116), pVH123 (SEQ ID NO: 117), pVH124 (SEQ ID NO: 118), pVH125 (SEQ ID NO: 119), pVH126 (SEQ ID NO: 120), pVH127 (SEQ ID NO: 121), pVH128 (SEQ ID NO: 122), pVH129 (SEQ ID NO: 123), and pVH130 (SEQ ID NO: 124). Thereafter, a human antibody heavy chain pseudogene sequence pVH30 (SEQ ID NO: 125) was designed by ligating these sequences to one another, and was then subjected to gene synthesis.

### (2) Left Arm

The Left Arm described in 1-6 above was used.

### (3) Right Arm

The Right Arm described in 1-6 above was used.

### (4) Multi-Cloning Site

For insertion of the human antibody heavy chain pseudogene sequence, a multi-cloning site shown in SEQ ID NO: 65 was designed.

### (5) Construction of Targeting Vector

The Left Arm, neomycin resistance gene, multi-cloning site, Right Arm, human IgG₁ variable region were incorporated into pUC19, and thereafter, the human antibody heavy chain pseudogene region pVH30 was incorporated into the multi-cloning site, so as to produce a targeting vector pVH KI_pUC19_step3_pVH#odd_even (SEQ ID NO: 126) as shown in FIG. 24.

### 17. Production of Cell Lines

The Lambda-VL-CL KI vector 2.0 produced in 12-1 above, the pVL KI_pUC19_step5_BrRev produced in 12-2 above, the pVL KI_pUC19_step6_BrRev_pVL#odd produced in 12-3 above, the human CH KI vector produced in 12-4 above, the pVH KI_pUC19_step5_BsrRev_RMCE_cassette produced in 16-5 above, and the pVH KI_pUC19_step3_pVH#odd_even produced in 16-6 above were each introduced into DT40 cells, so as to produce cell lines.

First, the human CH KI vector, which had been linearized with the restriction enzyme SalI, was introduced into the cells, and the obtained mixture was then cultured in a medium containing 2 mg/mL G418. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

Subsequently, the Lambda-VL-CL KI vector 2.0, which had been linearized with the restriction enzyme NotI, was introduced into DT40 cells, and the obtained mixture was then cultured in a medium containing 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance gene had been removed.

Thereafter, the pVL KI_pUC19_step5_BrRev, which had been linearized with the restriction enzyme NotI, was introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance genes had been removed.

Thereafter, the pVL KI_pUC19_step6_BrRev_pVL#odd, which had been linearized with the restriction enzyme NotI, was introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance genes had been removed.

Further, the pVH KI_pUC19_step5_BsrRev_RMCE_cassette, which had been linearized with the restriction enzyme NotI, was introduced into the cells, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to confirm that the drug resistance genes had been removed.

Finally, the pVH KI_pUC19_step3_pVH#odd_even, which had been linearized with the restriction enzyme NotI, was introduced into the cells, and the obtained mixture was then cultured in a medium containing 2 mg/mL G418. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to obtain cell lines #7-7, #11-3 and #11-6, from each of which the drug resistance gene had been removed.

### 18. Confirmation of Antibody Expression (Flow Cytometry and ELISA)

### 18-1. Analysis by Flow Cytometry

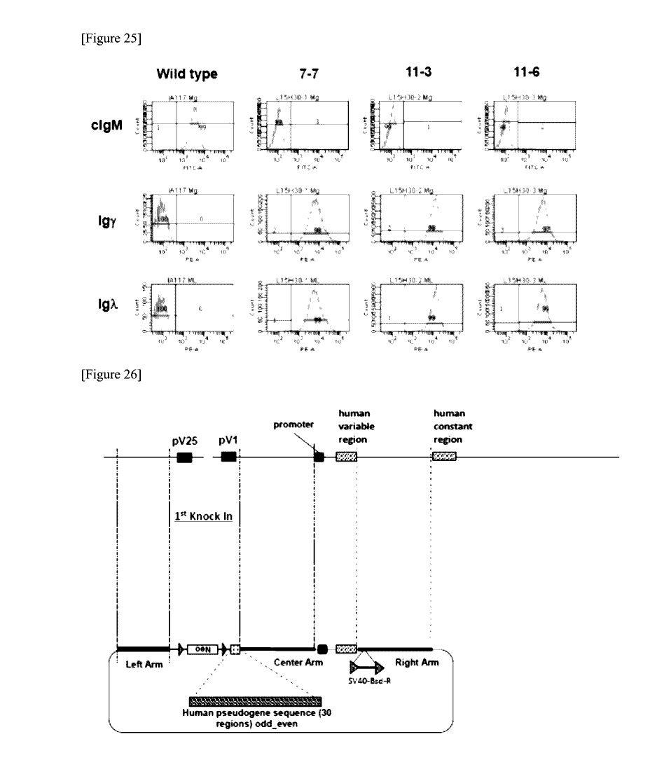

Based on the method described in 3 above, the expression of an antibody in the cells produced in 16 above was confirmed. The results are shown in FIG. 25. It was confirmed that only chicken IgM was expressed in wild-type DT40, whereas the expression of chicken IgM disappeared and instead, human Igγ and Igλ were expressed in the transformed cell lines #7-7, #11-3 and #11-6 (FIG. 25).

### 18-2. Analysis by ELISA

The antibodies secreted from the cells produced in 17 above were measured. The measurement of human IgG was carried out based on the method described in 3-2 above. The method for measuring chicken IgM is as follows.

That is, 20 μL of 1.0 μg/mL Goat anti-chicken IgM (Bethyl, A30-102A) was dispensed on an immuno 384-well plate Maxisorp (Nunc, 464718), and it was then reacted at room temperature for 1 hour or more, so that it was immobilized on the plate. Thereafter, the resultant was washed with a washing solution (PBS containing 0.05% Tween 20) five times, and 50 μL of a blocking solution (PBS containing 1% BSA) was then added thereto. The obtained mixture was reacted at room temperature for 30 minutes. Thereafter, the resultant was washed with a washing solution five times, and 20 μL of a measurement sample or chicken serum (GIBCO, 16110) serving as a standard substance was then added thereto. The obtained mixture was reacted at room temperature for 1 hour. The resultant was washed with a washing solution five times, and 20 μL of Goat anti-chicken IgM HRP conjugated (Bethyl, A30-102P), which had been 5000 times diluted with PBS containing 1% BSA and 0.05% Tween 20, was then added thereto. The obtained mixture was reacted at room temperature for 1 hour. Thereafter, the reaction mixture was washed with a washing solution five times, 20 μL of TMB+(Dako, 5159985) was then added thereto, and the obtained mixture was subjected to a coloring reaction at room temperature for 3 minutes. Subsequently, 20 μL of 1 N sulfuric acid was added to the reaction mixture to termination the reaction. Using Infinite M1000 (TECAN), the absorbance at 450 nm was measured.

The results are shown in Table 15. It was confirmed that, as with 13-1 above, only chicken IgM was secreted from wild-type DT40, whereas human IgG₁ was secreted from the transformed cell lines #7-7, #11-3 and #11-6.

TABLE 15

| | Human IgG1 (μg/mL) | Chicken IgM (μg/mL) |
|---|---|---|
| Wild type | <0.01 | 2.33 |
| #7-7 | 0.13 | <0.01 |
| #11-3 | 0.24 | <0.01 |
| #11-6 | 0.16 | <0.01 |

### 19. Confirmation of Gene Conversion

Using the cell lines in which the expression of an antibody had been confirmed in 10 above, gene conversion was confirmed. Using, as a template, the genomic DNA extracted from the cells, which had been cultured for 42 days in the presence of TSA, by the method described in 4 above, a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56) and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line #11-6 (the top 10) are shown in Table 16 and Table 17, respectively. A clone comprising a the additionally inserted human pseudogene-derived sequence was found in the variable regions of both of the light chain and the heavy chain, and it was demonstrated that more various sequences than in the case of L15/H15 cell line were generated as a result of gene conversion. In addition, it was also confirmed that a variety of sequences were generated as a result of gene conversion even in other cell lines.

TABLE 16

| # | % | CDR1 | CDR2 | CDR3 |
|---|---|---|---|---|
| 1 | 0.166 | VL | pVL105 | VL |
| 2 | 0.166 | pVL105 | VL | VL |
| 3 | 0.125 | pVL113 | pVL113 | pVL113 |
| 4 | 0.125 | VL | pVL109 | VL |
| 5 | 0.083 | Not matched | pVL105 | VL |
| 6 | 0.083 | VL | pVL104 | pVL104 |
| 7 | 0.083 | VL | pVL113 | VL |
| 8 | 0.083 | pVL114 | pVL114 | pVL114 |
| 9 | 0.042 | pVL109 | pVL109 | pVL109 |
| 10 | 0.042 | pVL105 | pVL115 | pVL115 |

VL: sequence inserted into variable region
pVL: sequence inserted into pseudogene region
Not matched: sequence that is not completely matched with either VL or pVL (which is probably caused by partial genetic recombination or mutation)

TABLE 17

| # | % | CDR1 | CDR2 | CR3 |
|---|---|---|---|---|
| 1 | 1.077 | pVH123 | VH | VH |
| 2 | 0.824 | VH | pVH129 | VH |
| 3 | 0.760 | pVH104 | Not matched | VH |
| 4 | 0.507 | pVH123 | Not matched | VH |
| 5 | 0.444 | pVH117 | Not matched | VH |
| 6 | 0.380 | pVH129 | pVH129 | VH |
| 7 | 0.380 | pVH126 | pVH112 | VH |
| 8 | 0.380 | VH | pVH111 | Not matched |
| 9 | 0.317 | pVH102 | VH | Not matched |
| 10 | 0.253 | pVH112 | Not matched | VH |

VH: sequence inserted into variable region
pVH: sequence inserted into pseudogene region
Not matched: sequence that is not completely matched with either VH or pVH (which is probably caused by partial genetic recombination or mutation)

Production of L30/H30 Cell Line, in which 30 Pseudogene Regions Each Consisting of Human Variable Region-Derived Sequence have been Inserted into Light Chain, and 30 Such Pseudogene Regions have been Inserted into Heavy Chain
20. Knocking-in into Chicken Antibody Light Chain (Igλ) Pseudogene and Variable Regions
(1) Human Antibody Light Chain Pseudogene Sequence (pVL30)

Based on the sequences of human immunoglobulin λ variable regions in the database, 30 CDR1, CDR2 and CDR3 sequences were selected, and the framework sequence of the human Igλ variable region cloned in 1-1 above was then combined therewith, so as to design 30 sequences, namely, pVL101 (SEQ ID NO: 74), pVL102 (SEQ ID NO: 75), pVL103 (SEQ ID NO: 76), pVL104 (SEQ ID NO: 77), pVL105 (SEQ ID NO: 78), pVL106 (SEQ ID NO: 79), pVL107 (SEQ ID NO: 80), pVL108 (SEQ ID NO: 81), pVL109 (SEQ ID NO: 82), pVL110 (SEQ ID NO: 83), pVL111 (SEQ ID NO: 84), pVL112 (SEQ ID NO: 85), pVL113 (SEQ ID NO: 86), pVL114 (SEQ ID NO: 87), pVL115 (SEQ ID NO: 88), pVL116 (SEQ ID NO: 127), pVL117 (SEQ ID NO: 128), pVL118 (SEQ ID NO: 129), pVL119 (SEQ ID NO: 130), pVL120 (SEQ ID NO: 131), pVL121 (SEQ ID NO: 132), pVL122 (SEQ ID NO: 133), pVL123 (SEQ ID NO: 134), pVL124 (SEQ ID NO: 135), pVL125 (SEQ ID NO: 136), pVL126 (SEQ ID NO: 137), pVL127 (SEQ ID NO: 138), pVL128 (SEQ ID NO: 139), pVL129 (SEQ ID NO: 140), and pVL130 (SEQ ID NO: 141). Thereafter, a human antibody light chain pseudogene sequence pVL30 (SEQ ID NO: 142) comprising these sequences was designed, and was then subjected to gene synthesis.

### (2) Construction of Targeting Vector

The human pseudogene sequence pVL30 synthesized in the above section was incorporated into the multi-cloning site of the pVL KI_pUC19_step6_BrRev in 16-2 above, so as to construct a targeting vector pVL KI_pUC19_step6_BrRev_#odd+even (SEQ ID NO: 143) as shown in FIG. 26.

### 21. Production of Cell Lines

The pVL KI_pUC19_step6_BrRev_#odd+even prepared in 20 above was introduced into the L15/H30 cell line produced in 17 above to produce cell lines.

The pVL KI_pUC19_step6_BrRev_#odd+even, which had been linearized with the restriction enzyme NotI, was introduced into the aforementioned cell line, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to obtain cell lines #7-7-48-5, #7-7-48-7 and #7-7-48-10, from each of which the drug resistance genes had been removed.

### 22. Confirmation of Antibody Expression (Flow Cytometry and ELISA)

### 22-1. Analysis by Flow Cytometry

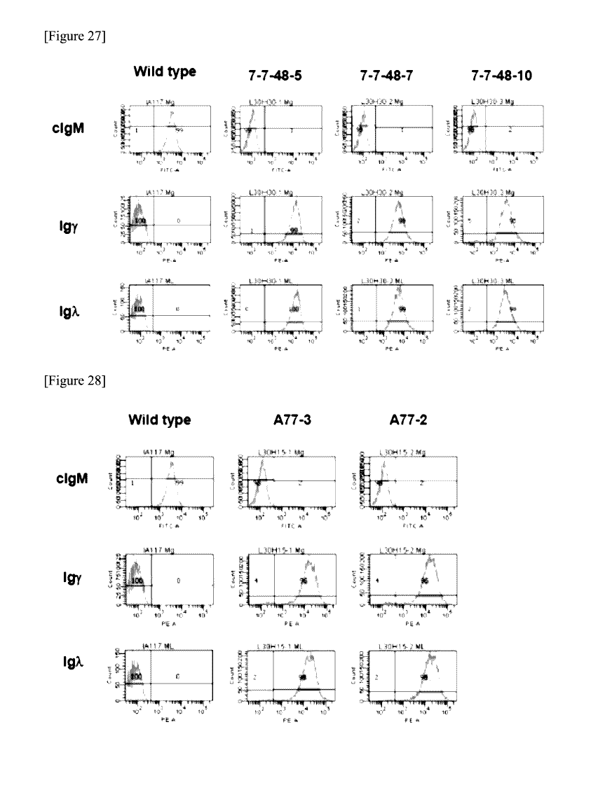

Based on the method described in 3 above, the expression of an antibody in the cells produced in 21 above was confirmed. The results are shown in FIG. 27. It was confirmed that only chicken IgM was expressed in wild-type DT40, whereas the expression of chicken IgM disappeared and instead, human Igγ and Igλ were expressed in the transformed cell lines #7-7-48-5, #7-7-48-7 and #7-7-48-10 (FIG. 27).

### 22-2. Analysis by ELISA

Based on the method described in 18-2 above, the antibodies secreted from the cells produced in 21 above were measured. The results are shown in Table 18. As with 13-1 above, it was confirmed that only chicken IgM was secreted from wild-type DT40, whereas human IgG₁ was secreted from the transformed cell lines #7-7-48-5, #7-7-48-7 and #7-7-48-10.

TABLE 18

| | Human IgG1 (μg/mL) | Chicken IgM (μg/mL) |
|---|---|---|
| Wild type | <0.01 | 2.33 |
| #7-7-48-5 | 0.26 | <0.01 |
| #7-7-48-7 | 0.16 | <0.01 |
| #7-7-48-10 | 0.13 | <0.01 |

### 23. Confirmation of Gene Conversion

Using the cell lines in which the expression of an antibody had been confirmed in 22 above, gene conversion was confirmed. Using, as a template, the genomic DNA extracted from the cells, which had been cultured for 42 days in the presence of TSA, by the method described in 4 above, a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56), and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line #7-7-48-10 (the top 10) are shown in Table 19 and Table 20, respectively. A clone comprising the additionally inserted human pseudogene-derived sequence was found in the variable regions of both of the light chain and the heavy chain, and it was demonstrated that more various sequences were generated by increasing the number of pseudogenes. In addition, it was also confirmed that a variety of sequences were generated as a result of gene conversion even in other cell lines.

TABLE 19

| # | % | CDR1 | CDR2 | CDR3 |
|---|---|---|---|---|
| 1 | 0.347* | VL | pVL118 | VL |
| 2 | 0.270 | pVL119 | VL | VL |
| 3 | 0.154 | VL | pVL123 | pVL118 |
| 4 | 0.116 | pVL130 | pVL130 | pVL130 |
| 5 | 0.116 | VL | VL | pVL127 |
| 6 | 0.077 | pVL116 | pVL116 | pVL116 |
| 7 | 0.077 | pVL119 | pVL119 | VL |
| 8 | 0.077 | VL | pVL129 | pVL129 |
| 9 | 0.077 | VL | pVL105 | VL |
| 10 | 0.077* | VL | pVL118 | VL |

VL: sequence inserted into variable region
pVL: sequence inserted into pseudogene region
Not matched: sequence that is not completely matched with either VL or pVL (which is probably caused by partial genetic recombination or mutation)
*Sequences of portions other than CDRs are different, respectively.

TABLE 20

| # | % | CDR1 | CDR2 | CDR3 |
|---|---|---|---|---|
| 1 | 33.191* | pVH104 | Not matched | VH |
| 2 | 8.085 | pVH129 | Not matched | VH |
| 3 | 6.241 | pVH108 | Not matched | VH |
| 4 | 5.106 | pVH111 | Not matched | VH |
| 5 | 3.688** | pVH126 | Not matched | VH |
| 6 | 1.418 | pVH126 | pVH126 | VH |
| 7 | 1.418 | pVH101 | Not matched | VH |
| 8 | 1.277* | pVH104 | Not matched | VH |
| 9 | 1.277** | pVH126 | Not matched | VH |
| 10 | 0.993* | pVH104 | Not matched | VH |

VH: sequence inserted into variable region
pVH: sequence inserted into pseudogene region
Not matched: sequence that is not completely matched with either VH or pVH (which is probably caused by partial genetic recombination or mutation)
*Sequences of CDR2 are different, respectively.
**Sequences of CDR2 are different, respectively.

Production of L30/H15 Cell Line, in which 30 Pseudogene Regions Each Consisting of Human Variable Region-Derived Sequence have been Inserted into Light Chain, and 15 Such Pseudogene Regions have been Inserted into Heavy Chain

### 24. Production of Cell Lines

The pVL KI_pUC19_step6_BrRev_#odd+even prepared in 20 above was introduced into the L15/H15 cell line A12-4 produced in 17 above to produce cell lines.

The pVL KI_pUC19_step6_BrRev_pVL#odd+even, which had been linearized with the restriction enzyme NotI, was introduced into the aforementioned cell line, and the obtained mixture was then cultured in a medium containing 2 mg/mL G418 and 10 μg/mL blasticidin. Thereafter, the growing cells were subjected to genotyping by PCR, so as to confirm that substitution had occurred in a gene locus of interest. Thereafter, Cre_pEGFP-C1 was introduced into the cells, and the resulting cells were then subjected to cell sorting to select GFP-positive cells. Thereafter, the selected cells were subjected to genotyping by PCR, so as to obtain cell lines A77-3 and A77-2, from each of which the drug resistance genes had been removed.

### 25. Confirmation of Antibody Expression (Flow Cytometry and ELISA)

### 25-1. Analysis by Flow Cytometry

Based on the method described in 3 above, the expression of an antibody in the cells produced in 24 above was confirmed. The results are shown in FIG. 28. It was confirmed that only chicken IgM was expressed in wild-type DT40, whereas the expression of chicken IgM disappeared and instead, human Igγ and Igλ were expressed in the transformed cell lines A77-3 and A77-2 (FIG. 28).

### 25-2. Analysis by ELISA

Based on the method described in 3-2 above, the antibodies secreted from the cells produced in 24 above were measured. The results are shown in Table 21. As with 13-1 above, it was confirmed that only chicken IgM was secreted from wild-type DT40, whereas human IgG₁ was secreted from the transformed cell lines A77-3 and A77-2.

TABLE 21

| | Human IgG1 (μg/mL) | Chicken IgM (μg/mL) |
|---|---|---|
| Wild type | <0.01 | 2.33 |
| A77-3 | 0.48 | <0.01 |
| A77-2 | 0.48 | <0.01 |

Production of L30/H15f15f and L30/H15r15f Cell Lines, in which 30 Pseudogene Regions Each Consisting of Human Variable Region-Derived Sequence have been Inserted into Light Chain and 30 Such Pseudogene Regions have been Inserted into Heavy Chain According to RMCE Method

### 26. Production of Vectors

26-1. Further Insertion of 15 Pseudogenes Constructed in Forward Direction into Chicken Antibody Heavy Chain (IgM) Pseudogene Region
(1) Human Antibody Heavy Chain Pseudogene Sequence (pVH15a)

The pVH116, pVH117, pVH118, pVH119, pVH120, pVH121, pVH122, pVH123, pVH124, pVH125, pVH126, pVH127, pVH128, pVH129 and pVH130, which had been designed in 16 above, were ligated to one another in the forward direction, so as to produce pVH15a (SEQ ID NO: 144).

### (2) Construction of Vector

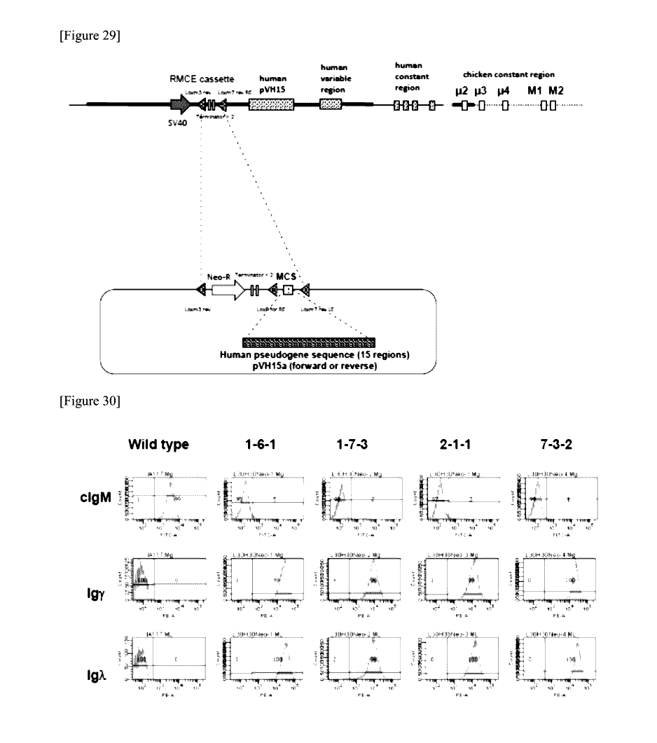

Loxm3 rev sequence, Neomycin resistance gene, SV40 early poly A terminator, SV40 polyadenylation region, loxP for RE sequence, pVH15a (forward direction), and loxm7 rev LE sequence were inserted into a pUC57-Amp vector, so as to produce a vector RMCE_pVH1stKI_pVH15evenFor (SEQ ID NO: 145) as shown in FIG. 29.

26-2. Further Insertion of 15 Pseudogenes Constructed in Reverse Direction into Chicken Antibody Heavy Chain (IgM) Pseudogene Region

Loxm3 rev sequence, Neomycin resistance gene, SV40 early poly A terminator, SV40 polyadenylation region, loxP for RE sequence, pVH15a (reverse direction), and loxm7 rev LE sequence were inserted into a pUC57-Amp vector, so as to produce a vector RMCE_pVH1stKI_pVH15evenRev (SEQ ID NO: 146) as shown in FIG. 29.

### 27. Production of Cell Lines

An L30/H15f15f cell line was produced by introducing the RMCE_pVH1stKI_pVH15evenFor produced in 26-1 above into the L30/H15 cell line produced in 24 above.

The RMCE_pVH1stKI_pVH15evenFor, together with Cre_pEGFP-C1, was introduced into the aforementioned cell line to induce site-specific recombination, and the obtained mixture was then cultured in a medium containing 2 mg/mL G418. Thereafter, the growing cells were subjected to genotyping by PCR, and cell lines 1-6-1 and 1-7-3 were obtained, in which substitution was confirmed to have occurred in a gene locus of interest.

An L30/H15r15f cell line was produced by introducing the RMCE_pVH1stKI_pVH15evenRev produced in 26-2 into the L30/H15 cell line produced in 24 above.

The RMCE_pVH1stKI_pVH15evenRev, together with Cre_pEGFP-C1, was introduced into the aforementioned cell line to induce site-specific recombination, and the obtained mixture was then cultured in a medium containing 2 mg/mL G418. Thereafter, the growing cells were subjected to genotyping by PCR, and cell lines 2-1-1 and 7-3-2 were obtained, in which substitution was confirmed to have occurred in a gene locus of interest.

### 28. Confirmation of Antibody Expression (Flow Cytometry and ELISA)

### 28-1. Analysis by Flow Cytometry

Based on the method described in 3 above, the expression of an antibody in the cells produced in 27 above was confirmed. The results are shown in FIG. 30. It was confirmed that only chicken IgM was expressed in wild-type DT40, whereas the expression of chicken IgM disappeared and instead, human Igγ and Igλ were expressed in the transformed cell lines 1-6-1, 1-7-3, 2-1-1 and 7-3-2 (FIG. 30).

### 28-2. Analysis by ELISA

Based on the method described in 3-2 above, the antibodies secreted from the cells produced in 27 above were measured. The results are shown in Table 22. As with 13-1 above, it was confirmed that only chicken IgM was secreted from wild-type DT40, whereas human IgG₁ was secreted from the transformed cell lines 1-6-1, 1-7-3, 2-1-1 and 7-3-2.

TABLE 22

| | Human IgG1 (μg/mL) | Chicken IgM (μg/mL) |
|---|---|---|
| Wild type | <0.01 | 2.33 |
| 1-6-1 | 0.18 | <0.01 |
| 1-7-3 | 0.07 | <0.01 |
| 2-1-1 | 0.12 | <0.01 |
| 7-3-2 | 0.18 | <0.01 |

### 29. Confirmation of Gene Conversion

Using the cell lines in which the expression of an antibody had been confirmed in 10 above, gene conversion was confirmed. Using, as a template, the genomic DNA extracted from the cells, which had been cultured for 42 days in the presence of TSA, by the method described in 4 above, a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56), and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line 1-6-1 (the top 10) are shown in Table 23 and Table 24, respectively, and the results of the sequence analysis performed on the variable regions of the light chain and heavy chain in the cell line 7-3-2 (the top 10) are shown in Table 25 and Table 26, respectively. A clone comprising the additionally inserted human pseudogene-derived sequence was found in the variable regions of both of the light chain and the heavy chain, and it was demonstrated that a variety of sequences were generated as in the case of the L30/H30 cell line. In addition, it was also confirmed that a variety of sequences were generated as a result of gene conversion even in other cell lines.

TABLE 23

| # | % | CDR1 | CDR2 | CDR3 |
|---|---|---|---|---|
| 1 | 0.935 | VL | pVL102 | VL |
| 2 | 0.468 | VL | pVL118 | VL |
| 3 | 0.271 | Not matched | pVL102 | VL |
| 4 | 0.271* | pVL119 | VL | VL |
| 5 | 0.222 | VL | VL | pVL129 |
| 6 | 0.172* | pVL119 | VL | VL |
| 7 | 0.172 | VL | pVL105 | VL |
| 8 | 0.123 | VL | pVL130 | VL |
| 9 | 0.098 | Not matched | pVL122 | VL |
| 10 | 0.098 | VL | pVL109 | VL |

VL: sequence inserted into variable region
pVL: sequence inserted into pseudogene region
Not matched: sequence that is not completely matched with either VL or pVL (which is probably caused by partial genetic recombination or mutation)
*Sequences of portions other than CDRs are different, respectively.

TABLE 24

| # | % | CDR1 | CDR2 | CDR3 |
|---|---|---|---|---|
| 1 | 6.393* | pVH112 | Not matched | VH |
| 2 | 0.586 | pVH109 | Not matched | VH |
| 3 | 0.479 | pVH108 | Not matched | VH |
| 4 | 0.479 | pVH117 | Not matched | VH |
| 5 | 0.426 | pVH107 | Not matched | VH |
| 6 | 0.320 | pVH124 | Not matched | VH |
| 7 | 0.266* | pVH112 | Not matched | VH |
| 8 | 0.266 | VH | Not matched | pVH109 |
| 9 | 0.160 | Not matched | pVH125 | VH |
| 10 | 0.160* | pVH112 | Not matched | VH |

VH: sequence inserted into variable region
pVH: sequence inserted into pseudogene region
Not matched: sequence that is not completely matched with either VH or pVH (which is probably caused by partial genetic recombination or mutation)
*Sequences of CDR2 are different, respectively.

TABLE 25

| # | % | CDR1 | CDR2 | CDR3 |
|---|---|---|---|---|
| 1 | 0.293 | VL | pVL109 | VL |
| 2 | 0.267 | pVL119 | VL | VL |
| 3 | 0.187* | pVL105 | VL | VL |
| 4 | 0.160 | VL | VL | pVL129 |
| 5 | 0.133 | VL | pVL101 | VL |
| 6 | 0.133 | VL | pVL118 | VL |
| 7 | 0.133 | VL | VL | pVL127 |
| 8 | 0.107 | pVL119 | pVL119 | VL |
| 9 | 0.080 | VL | VL | pVL123 |
| 10 | 0.080* | pVL105 | VL | VL |

VL: sequence inserted into variable region
pVL: sequence inserted into pseudogene region
Not matched: sequence that is not completely matched with either VL or pVL (which is probably caused by partial genetic recombination or mutation)
*Sequences of portions other than CDRs are different, respectively.

TABLE 26

| # | % | CDR1 | CDR2 | CDR3 |
|---|---|---|---|---|
| 1 | 0.867* | VH | Not matched | pVH109 |
| 2 | 0.819* | VH | Not matched | pVH109 |
| 3 | 0.819 | VH | Not matched | pVH116 |
| 4 | 0.337 | pVH109 | Not matched | VH |
| 5 | 0.241* | VH | Not matched | pVH109 |
| 6 | 0.193 | pVH105 | Not matched | VH |
| 7 | 0.193 | pVH117 | Not matched | VH |
| 8 | 0.145 | pVH112 | Not matched | VH |
| 9 | 0.145 | pVH126 | Not matched | pVH116 |
| 10 | 0.096 | pVH107 | Not matched | pVH109 |

VH: sequence inserted into variable region
pVH: sequence inserted into pseudogene region
Not matched: sequence that is not completely matched with either VH or pVH (which is probably caused by partial genetic recombination or mutation)
*Sequences of CDR2 are different, respectively.

Production of L30/H15f15f15f and L30/H15r15r15f Cell Lines in which 30 Pseudogene Regions Each Consisting of Human Variable Region-Derived Sequence have been Inserted into Light Chain and 45 Such Pseudogene Regions have been Inserted into Heavy Chain According to RMCE Method

### 30. Production of Vector

30-1. Further Insertion of 15 Pseudogenes Constructed in Forward Direction into Chicken Antibody Heavy Chain (IgM) Pseudogene Region
(1) Human Antibody Heavy Chain Pseudogene Sequence (pVH15b)

Based on the sequences of a human immunoglobulin heavy chain variable regions in the database, novel 15 CDR1, CDR2 and CDR 3 sequences were selected, and pVH131 (SEQ ID NO: 147), pVH132 (SEQ ID NO: 148), pVH133 (SEQ ID NO: 149), pVH134 (SEQ ID NO: 150), pVH135 (SEQ ID NO: 151), pVH136 (SEQ ID NO: 152), pVH137 (SEQ ID NO: 153), pVH138 (SEQ ID NO: 154), pVH139 (SEQ ID NO: 155), pVH140 (SEQ ID NO: 156), pVH141 (SEQ ID NO: 157), pVH142 (SEQ ID NO: 158), pVH143 (SEQ ID NO: 159), pVH144 (SEQ ID NO: 160), and pVH145 (SEQ ID NO: 161) were ligated to one another in the forward direction to produce pVH15b (SEQ ID NO: 162).

### (2) Construction of Vector

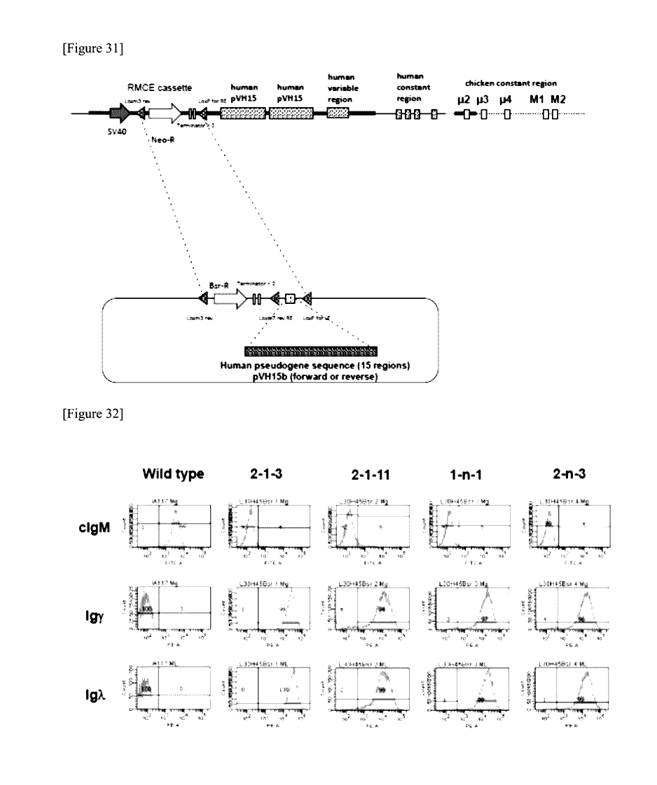

Loxm3 rev sequence, blasticidin resistance gene, SV40 early poly A terminator, SV40 polyadenylation region, loxm7 rev RE sequence, pVH15b (forward direction), and loxP for LE sequence were inserted into a pUC57-Amp vector, so as to produce a vector pVH15B-ver3_RMCE2nd_For (SEQ ID NO: 163) as shown in FIG. 31.

30-2. Further Insertion of 15 Pseudogenes Constructed in Reverse Direction into Chicken Antibody Heavy Chain (IgM) Pseudogene Region

Loxm3 rev sequence, blasticidin resistance gene, SV40 early poly A terminator, SV40 polyadenylation region, loxm7 rev RE sequence, pVH15b (reverse direction), and loxP for LE sequence were inserted into a pUC57-Amp vector, so as to produce a vector pVH15B-ver3_RMCE2nd_Rev (SEQ ID NO: 164) as shown in FIG. 31.

### 31. Production of Cell Lines

An L30/H15f15f15f cell line was produced by introducing the pVH15B-ver3_RMCE2nd_For produced in 30-1 above into the L30/H15f15f cell line produced in 2 above.

The pVH15B-ver3_RMCE2nd_For, together with Cre_pEGFP-C1, was introduced into the aforementioned cell line to induce site-specific recombination, and the obtained mixture was then cultured in a medium containing 10 μg/mL blasticidin. Thereafter, the growing cells were subjected to genotyping by PCR, and cell lines 2-1-3, 2-1-11, 1-n-1 and 2-n-3 were obtained, in which substitution was confirmed to have occurred in a gene locus of interest.

An L30/H15r15r15f cell line was produced by introducing the pVH15B-ver3_RMCE2nd_Rev produced in 27-2 above into the L30/H15r15f cell line produced in 27 above.

The pVH15B-ver3_RMCE2nd_Rev, together with Cre_pEGFP-C1, was introduced into the aforementioned cell line to induce site-specific recombination, and the obtained mixture was then cultured in a medium containing 10 μg/mL blasticidin. Thereafter, the growing cells were subjected to genotyping by PCR, and cell lines 4-1-5, 4-1-7 and 3-1-11 were obtained, in which substitution was confirmed to have occurred in a gene locus of interest.

### 32. Confirmation of Antibody Expression (Flow Cytometry and ELISA)

### 32-1. Analysis by Flow Cytometry

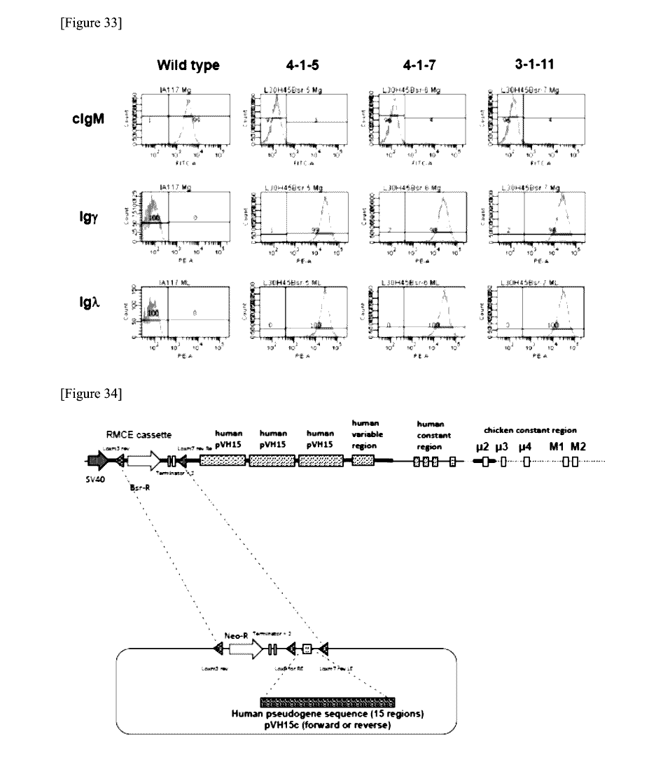

Based on the method described in 3 above, the expression of an antibody in the cells produced in 31 above was confirmed. The results are shown in FIG. 32 and FIG. 33. It was confirmed that only chicken IgM was expressed in wild-type DT40, whereas the expression of chicken IgM disappeared and instead, human Igγ and Igλ were expressed in the transformed cell lines 2-1-3, 2-1-11, 1-n-1 and 2-n-3 (which are shown in FIG. 32), and in the transformed cell lines 4-1-5, 4-1-7 and 3-1-11 (which are shown in FIG. 33).

### 32-2. Analysis by ELISA

Based on the method described in 3-2 above, the antibodies secreted from the cells produced in 31 above were measured. The results are shown in Table 27. As with 13-1 above, it was confirmed that only chicken IgM was secreted from wild-type DT40, whereas human IgG₁ was secreted from the transformed cell lines 2-1-3, 2-1-11, 1-n-1, 2-n-3, 4-1-5, 4-1-7 and 3-1-11.

TABLE 27

| | Human IgG1 (μg/mL) | Chicken IgM (μg/mL) |
|---|---|---|
| Wild type | <0.01 | 2.33 |
| 2-1-3 | 0.36 | <0.01 |
| 2-1-11 | 0.16 | <0.01 |
| 1-n-1 | 0.18 | <0.01 |
| 2-n-3 | 0.16 | <0.01 |
| 4-1-5 | 0.37 | <0.01 |
| 4-1-7 | 0.94 | <0.01 |
| 3-1-11 | 1.31 | <0.01 |

### 33. Confirmation of Gene Conversion

Using the cell lines in which the expression of an antibody had been confirmed in 10 above, gene conversion was confirmed. Using, as a template, the genomic DNA extracted from the cells, which had been cultured for 42 days in the presence of TSA, by the method described in 4 above, a light chain variable region was amplified by PCR using a sense primer to which a recognition sequence had been added (CGTATCGCCTCCCTCGCGCCATCAGNNNNNNNNNNCAGGTTCCCTGGTGCAGGC) (SEQ ID NO: 46), d, and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56), and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line 2-1-3 (the top 10) are shown in Table 28 and Table 29, and the results of the sequence analysis performed on the variable regions of the light chain and heavy chain in the cell line 3-1-11 (the top 10) are shown in Table 30 and Table 31, respectively. A clone comprising the additionally inserted human pseudogene-derived sequence was found in the variable regions of both of the light chain and the heavy chain, and it was demonstrated that a variety of sequences were generated as a result of gene conversion. In addition, it was also confirmed that a variety of sequences were generated as a result of gene conversion even in other cell lines.

TABLE 28

| # | % | CDR1 | CDR2 | CDR3 |
|---|---|---|---|---|
| 1 | 0.882* | pVL119 | VL | VL |
| 2 | 0.428** | VL | pVL118 | VL |
| 3 | 0.241 | pVL119 | pVL119 | VL |
| 4 | 0.187** | VL | pVL118 | VL |
| 5 | 0.187** | VL | pVL118 | VL |
| 6 | 0.134 | VL | pVL119 | VL |
| 7 | 0.134 | VL | pVL109 | VL |
| 8 | 0.134 | pVL105 | VL | VL |
| 9 | 0.107* | pVL119 | VL | VL |
| 10 | 0.080 | VL | pVL105 | VL |

VL: sequence inserted into variable region
pVL: sequence inserted into pseudogene region
Not matched: sequence that is not completely matched with either VL or pVL (which is probably caused by partial genetic recombination or mutation)
*, **Sequences of portions other than CDRs are different, respectively.

TABLE 29

| # | % | CDR1 | CDR2 | CDR3 |
|---|---|---|---|---|
| 1 | 2.226 | pVH112 | Not matched | VH |
| 2 | 1.272 | pVH109 | Not matched | VH |
| 3 | 0.636 | VH | pVH112 | VH |
| 4 | 0.477 | pVH144 | Not matched | VH |
| 5 | 0.477 | pVH105 | Not matched | VH |
| 6 | 0.318 | pVH145 | Not matched | VH |
| 7 | 0.318 | pVH137 | Not matched | VH |
| 8 | 0.318* | pVH108 | Not matched | VH |
| 9 | 0.318* | pVH108 | Not matched | VH |
| 10 | 0.318 | VH | Not matched | pVH116 |

VH: sequence inserted into variable region
pVH: sequence inserted into pseudogene region
Not matched: sequence that is not completely matched with etiher VH or pVH (which is probably caused by partial genetic recombination or mutation)
*Sequences of CDR2 are different, respectively.

TABLE 30

| # | % | CDR1 | CDR2 | CDR3 |
|---|---|---|---|---|
| 1 | 1.000 | pVL105 | VL | VL |
| 2 | 0.788 | pVL119 | VL | VL |
| 3 | 0.515* | VL | pVL118 | VL |
| 4 | 0.455* | VL | pVL118 | VL |
| 5 | 0.182 | VL | pVL105 | VL |
| 6 | 0.152** | pVL119 | pVL119 | VL |
| 7 | 0.152 | VL | pVL109 | VL |
| 8 | 0.121 | VL | pVL104 | VL |
| 9 | 0.091** | pVL119 | pVL119 | VL |
| 10 | 0.091 | VL | pVL107 | VL |

VL: sequence inserted into variable region
pVL: sequence inserted into pseudogene region
Not matched: sequence that is not completely matched with either VL or pVL (which is probably caused by partial genetic recombination or mutation)
*, **Sequences of portions other than CDRs are different, respectively.

TABLE 31

| # | % | CDR1 | CDR2 | CDR3 |
|---|---|---|---|---|
| 1 | 1.524 | pVH109 | Not matched | VH |
| 2 | 1.220 | pVH112 | Not matched | VH |
| 3 | 0.915 | pVH117 | Not matched | VH |
| 4 | 0.915 | pVH142 | Not matched | VH |
| 5 | 0.610 | pVH108 | Not matched | VH |
| 6 | 0.610 | pVH138 | Not matched | VH |
| 7 | 0.610 | pVH107 | Not matched | VH |
| 8 | 0.457 | pVH139 | Not matched | VH |
| 9 | 0.457 | pVH102 | Not matched | VH |
| 10 | 0.457 | pVH142 | Not matched | Not matched |

VH: sequence inserted into variable region
pVH: sequence inserted into pseudogene region
Not matched: sequence that is not completely matched with either VH or pVH (which is probably caused by partial genetic recombination or mutation)

Production of L30/H15f15f15f15f and L30/H15r15r15r15f Cell Lines, in which 30 Pseudogene Regions Each Consisting of Human Variable Region-Derived Sequence have been Inserted into Light Chain and 60 Such Pseudogene Regions have been Inserted into Heavy Chain According to RMCE Method

### 34. Production of Vector

34-1. Further Insertion of 15 Pseudogenes Constructed in Forward Direction into Chicken Antibody Heavy Chain (IgM) Pseudogene Region
(1) Human Antibody Heavy Chain Pseudogene Sequence (pVH15c)

Based on the sequences of human immunoglobulin heavy chain variable regions in the database, novel 15 CDR1, CDR2 and CDR 3 sequences were selected, and pVH146 (SEQ ID NO: 165), pVH147 (SEQ ID NO: 166), pVH148 (SEQ ID NO: 167), pVH149 (SEQ ID NO: 168), pVH150 (SEQ ID NO: 169), pVH151 (SEQ ID NO: 170), pVH152 (SEQ ID NO: 171), pVH153 (SEQ ID NO: 172), pVH154 (SEQ ID NO: 173), pVH155 (SEQ ID NO: 174), pVH156 (SEQ ID NO: 175), pVH157 (SEQ ID NO: 176), pVH158 (SEQ ID NO: 177), pVH159 (SEQ ID NO: 178), and pVH160 (SEQ ID NO: 179) were ligated to one another in the forward direction to produce pVH15c (SEQ ID NO: 180).

### (2) Construction of Vector

Loxm3 rev sequence, neomycin resistance gene, SV40 early poly A terminator, SV40 polyadenylation region, loxP for RE sequence, pVH15c (forward direction), and loxm7 rev LE sequence were inserted into a pUC57-Amp vector, so as to produce a vector pVH15A_RMCE1st_Fw (SEQ ID NO: 181) as shown in FIG. 34.

34-2. Further Insertion of 15 Pseudogenes Constructed in Reverse Direction into Chicken Antibody Heavy Chain (IgM) Pseudogene Region

Loxm3 rev sequence, neomycin resistance gene, SV40 early poly A terminator, SV40 polyadenylation region, loxP for RE sequence, pVH15c (reverse direction), and loxm7 rev LE sequence were inserted into a pUC57-Amp vector, so as to produce a vector pVH15A_RMCE1st_Rv (SEQ ID NO: 182) as shown in FIG. 34.

### 35. Production of Cell Lines

An L30/H15f15f15f15f cell line was produced by introducing the pVH15A_RMCE1st_Fw produced in 34-1 above into the L30/H15f15f15f cell line produced in 31 above.

The pVH15A_RMCE1st_Fw, together with Cre_pEGFP-C1, was introduced into the aforementioned cell line to induce site-specific recombination, and the obtained mixture was then cultured in a medium containing 2 mg/mL G418. Thereafter, the growing cells were subjected to genotyping by PCR, and cell lines 1n1-3, 1n1-4, and 1n1-5 were obtained, in which substitution was confirmed to have occurred in a gene locus of interest.

An L30/H15r15r15r15f cell line was produced by introducing the pVH15A_RMCE1st_Rv produced in 34-2 above into the L30/H15r15r15f cell line produced in 31 above.

The pVH15A_RMCE1st_Rv, together with Cre_pEGFP-C1, was introduced into the aforementioned cell line to induce site-specific recombination, and the obtained mixture was then cultured in a medium containing 2 mg/mL G418. Thereafter, the growing cells were subjected to genotyping by PCR, and a cell line 3111-1 was obtained, in which substitution was confirmed to have occurred in a gene locus of interest.

### 36. Confirmation of Antibody Expression (Flow Cytometry and ELISA)

### 36-1. Analysis by Flow Cytometry

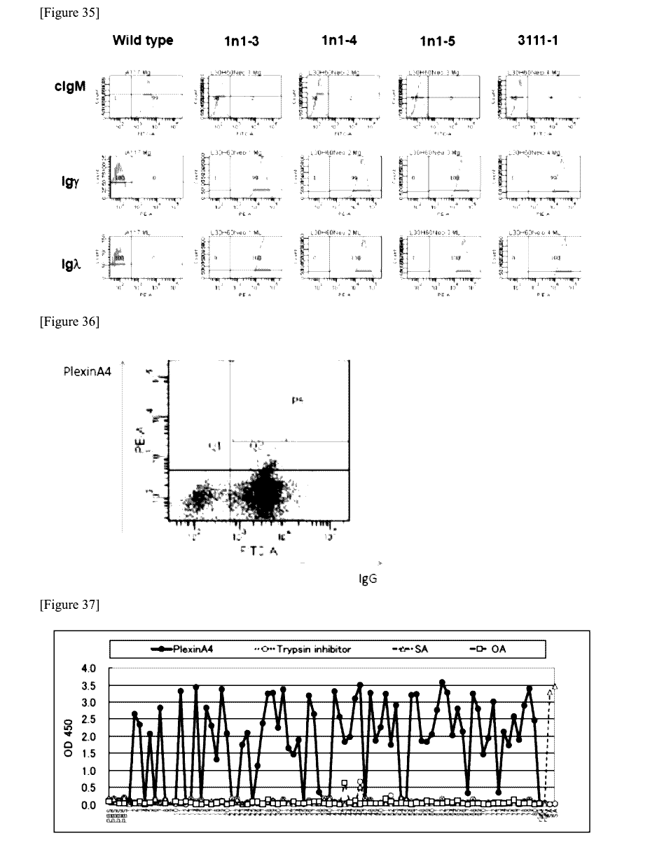

Based on the method described in 3 above, the expression of an antibody in the cells produced in 35 above was confirmed. The results are shown in FIG. 35. It was confirmed that only chicken IgM was expressed in wild-type DT40, whereas the expression of chicken IgM disappeared and instead, human Igγ and Igλ were expressed in the transformed cell lines 1n1-3, 1n1-4, 1n1-5 and 3111-1 (FIG. 35).

### 36-2. Analysis by ELISA

Based on the method described in 3-2 above, the antibodies secreted from the cells produced in 35 above were measured. The results are shown in Table 32. As with 13-1 above, it was confirmed that only chicken IgM was secreted from wild-type DT40, whereas human IgG₁ was secreted from the transformed cell lines 1n1-3, 1n1-4, 1n1-5 and 3111-1.

TABLE 32

| | Human IgG1 (μg/mL) | Chicken IgM (μg/mL) |
|---|---|---|
| Wild type | <0.01 | 2.33 |
| 1n1-3 | 0.32 | <0.01 |
| 1n1-4 | 0.34 | <0.01 |
| 1n1-5 | 0.22 | <0.01 |
| 3111-1 | 0.49 | <0.01 |

### 37. Confirmation of Gene Conversion

Using the cell lines in which the expression of an antibody had been confirmed in 10 above, gene conversion was confirmed. Using, as a template, the genomic DNA extracted from the cells, which had been cultured for 42 days in the presence of TSA, by the method described in 4 above, a light chain variable region was amplified by PCR using a sense primer to which a recognition sequence had been added (GTATCGCCTCCCTCGCGCCATCAGNNNNNNNNNNCAGGTTCCCTGGTGCAGGC) (SEQ ID NO: 46), and an antisense primer (CTATGCGCCTTGCCAGCCCGCTCAGGCTTGGTCCCTCCGCCGAA) (SEQ ID NO: 47), and a heavy chain variable region was amplified by PCR using a sense primer (CTATGCGCCTTGCCAGCCCGCTCAGTCCGTCAGCGCTCTCT) (SEQ ID NO: 56), and an antisense primer to which an identification tag had been added (CGTATCGCCTCCCTCGCGCCATCAGNNNNNNNNNNTGGGGGGGGTTCATATGAAG) (SEQ ID NO: 57). Thereafter, sequence analyses were carried out based on the method described in 4 above. The results of the sequence analysis performed on the variable regions of the light chain and heavy chain in the cell line 1n1-3 (the top 10) are shown in Table 33 and Table 34, and the results of the sequence analysis performed on the variable regions of the light chain and heavy chain in the cell line 3111-1 (the top 10) are shown in Table 35 and Table 36, respectively. A clone comprising the additionally inserted human pseudogene-derived sequence was found in the variable regions of both of the light chain and the heavy chain, and it was demonstrated that more various sequences were generated as a result of gene conversion. In addition, it was also confirmed that a variety of sequences were generated as a result of gene conversion even in other cell lines.

TABLE 33

| # | % | CDR1 | CDR2 | CDR3 |
|---|---|---|---|---|
| 1 | 1.217 | Not matched | pVL105 | VL |
| 2 | 0.466* | Not matched | pVL118 | VL |
| 3 | 0.304 | pVL119 | VL | VL |
| 4 | 0.223** | pVL119 | pVL119 | VL |
| 5 | 0.223* | Not matched | pVL118 | VL |
| 6 | 0.162 | Not matched | pVL109 | VL |
| 7 | 0.162 | pVL105 | VL | VL |
| 8 | 0.142 | Not matched | pVL119 | VL |
| 9 | 0.081 | Not matched | VL | pVL129 |
| 10 | 0.061** | pVL119 | pVL119 | VL |

VL: sequence inserted into variable region
pVL: sequence inserted into pseudogene region
Not matched: sequence that is not completely matched with either VL or pVL (which is probably caused by partial genetic recombination or mutation)
*Sequences of CDR2 are different, respectively
**Sequences of portions other than CDRs are different, respectively.

TABLE 34

| # | % | CDR1 | CDR2 | CDR3 |
|---|---|---|---|---|
| 1 | 63.115* | pVH105 | Not matched | VH |
| 2 | 2.186 | pVH138 | pVH138 | VH |
| 3 | 0.929 | pVH124 | Not matched | VH |
| 4 | 0.820 | pVH112 | Not matched | VH |
| 5 | 0.765* | pVH105 | Not matched | VH |
| 6 | 0.656* | pVH105 | Not matched | VH |
| 7 | 0.601 | pVH155 | Not matched | VH |
| 8 | 0.601* | pVH105 | Not matched | VH |
| 9 | 0.546* | pVH105 | Not matched | VH |
| 10 | 0.492 | pVH156 | Not matched | VH |

VH: sequence inserted into variable region
pVH: sequence inserted into pseudogene region
Not matched: sequence that is not completely matched with either VH or pVH (which is probably caused by partial genetic recombination or mutation)
*Sequences of CDR2 are different, respectively.

TABLE 35

| # | % | CDR1 | CDR2 | CDR3 |
|---|---|---|---|---|
| 1 | 0.142 | VL | VL | pVL129 |
| 2 | 0.094 | VL | VL | pVL128 |
| 3 | 0.071* | pVL119 | VL | VL |
| 4 | 0.047* | pVL119 | VL | VL |
| 5 | 0.024 | Not matched | pVL107 | pVL107 |
| 6 | 0.024 | pVL108 | pVL123 | pVL123 |
| 7 | 0.024 | pVL108 | pVL108 | pVL123 |
| 8 | 0.024* | pVL119 | VL | VL |
| 9 | 0.024 | VL | VL | pVL125 |
| 10 | 0.024 | VL | VL | pVL130 |

VL: sequence inserted into variable region
pVL: sequence inserted into pseudogene region
Not matched: sequence that is not completely matched with either VL or pVL (which is probably caused by partial genetic recombination or mutation)
*Sequences of portions other than CDRs are different, respectively.

TABLE 36

| # | % | CDR1 | CDR2 | CDR3 |
|---|---|---|---|---|
| 1 | 51.762* | pVH126 | Not matched | VH |
| 2 | 23.429 | pVH108 | Not matched | VH |
| 3 | 1.053 | pVH129 | Not matched | VH |
| 4 | 0.545* | pVH126 | Not matched | VH |
| 5 | 0.436* | pVH126 | Not matched | VH |
| 6 | 0.291 | pVH104 | Not matched | VH |
| 7 | 0.291* | pVH126 | Not matched | VH |
| 8 | 0.291** | pVH126 | Not matched | Not matched |
| 9 | 0.254** | pVH126 | Not matched | Not matched |
| 10 | 0.254* | pVH126 | Not matched | VH |

VH: sequence inserted into variable region
pVH: sequence inserted into pseudogene region
Not matched: sequence that is not completely matched with either VH or pVH (which is probably caused by partial genetic recombination or mutation)
*Sequences of CDR2 are different, respectively.
*Sequences of CDR2 and CDR3 are different, respectively.

### Antibody Selection 1

### 38. Preparation of Antigen

Plexin A4 was selected as an antigen protein. Plexin A4 is a molecule belonging to Plexin A type, and has been known as a factor for controlling axon extension in the sensory nerve or the sympathetic nerve.

As an antigen, Plexin A4, in which FLAG tags were added to the C-termini of a Sema domain and a PSI (Plexin-Semaphorin-Integrin) domain, was used (the amino acid sequence is shown in SEQ ID NO: 68). Based on Accession No. NM_020911 and Accession No. EAW83796, the nucleotide sequence shown in SEQ ID NO: 67 was synthesized, and it was then inserted into an expression vector in which the EBNA-1 gene and Hygromycin resistance gene of pCEP4 (Life Technologies, V044-50) had been deleted, thereby constructing a Plexin A4 expression vector.

The obtained Plexin A4 expression vector was introduced into Free Style 293 F cells (Life Technologies, R790-07), using PEI (polyethylenimine), so that it was allowed to express therein. At that time, an expression vector comprising an EBNA-1 gene was also introduced into the cells, separately. The expression was carried out at 3 L-scale, and the culture was carried out for 7 days.

Utilizing FLAG tags, a protein of interest in the culture supernatant was purified. An XK16/20 column (GE Healthcare, 28-9889-37) was filled with 2.5 mL of anti-M2-agarose resin (Sigma, A2220), and it was then equilibrated with D-PBS(−) (Wako Pure Chemical Industries, Ltd., 045-29795). Thereafter, the culture supernatant was loaded thereon, it was washed with D-PBS(−), and it was then eluted with D-PBS(−) containing 0.1 mg/mL FLAG peptide (Sigma, F3290). The resultant was monitored with the absorbance at 280 nm, and a fraction corresponding to the peak was then recovered. The molecular weight of the recovered protein was measured by SDS-PAGE and CBB staining, and it was then confirmed that the obtained molecular weight corresponded to the molecular weight of the protein of interest.

Using an ultrafiltration membrane (Amicon, UFC0801008), approximately 24 mL of the recovered fraction was concentrated to a volume of 2 mL, and then, using Hi Load 16/600 Superdex 200 pg (GE Healthcare, 28-9893-35), the fraction was purified by gel filtration chromatography. The column was equilibrated with D-PBS(−), and the recovered fraction was then loaded thereon, followed by elution with D-PBS(−). The resultant was monitored with the absorbance at 280 nm, and a fraction corresponding to the peak was then recovered. The molecular weight of the recovered protein was measured by SDS-PAGE and CBB staining.

### 39. Selection of Antibody

### 39-1. Production of Antigen Magnetic Beads

Using Dynabeads M-270 Carboxylic Acid (Life Technologies, 14305D) and Dynabeads M-270 Epoxy (Life Technologies, 14301) as magnetic beads, these beads were allowed to bind to the antigen in accordance with the instruction manual. At this time, MPC-S (Life Technologies, DBA13346) was used as a magnetic stand. Regarding Dynabeads M-270 Carboxylic Acid, 10 μL of beads were washed with 20 μL of 25 mM IVIES (pH 5.0) three times, and 10 μL of 50 mg/mL NHS solution and 10 μL of 50 mg/mL EDC solution were added to the resulting solution. The obtained mixture was reacted at room temperature for 30 minutes, and the reaction solution was then washed with 20 μL of 25 mM IVIES (pH 5.0) twice. A supernatant was removed from this magnetic beads suspension, and 10 μL of Plexin A4 protein solution, which had been adjusted to 0.6 mg/mL with 25 mM IVIES (pH 5.0), was then added to the suspension. The obtained mixture was reacted at 4° C. overnight, while being subjected to rotary stirring. Thereafter, a supernatant was removed from the reaction solution, and 20 μL of quenching buffer (50 mM Tris-HCl) was then added thereto, followed by the reaction of the mixture at room temperature for 15 minutes while being subjected to rotary stirring. Regarding Dynabeads M-270 Epoxy, 10 μL of beads were washed with 20 μL of 100 mM Na-phosphate buffer three times, and thereafter, 13.3 μL of Plexin A4 protein solution adjusted to 0.45 mg/ml with 100 mM Na-phosphate buffer and 6.7 μL of 100 mM Na-phosphate buffer containing 3 M ammonium sulfate were added and suspended therein. The obtained mixture was reacted at 37° C. overnight, while being subjected to rotary stirring. After the removal of a supernatant, the two types of beads were collectively suspended in 1 mL of selection buffer (PBS containing 1% BSA), and the obtained suspension was then washed with the same buffer as mentioned above three times. Thereafter, the resultant was suspended in 200 μL of selection buffer, and was then subjected to selection.

### 39-2. Selection by Antigen Magnetic Beads

An L15/H15 cell line, B7-3, was cultured in a medium containing 2.5 ng/mL TSA for 3 weeks. Thereafter, approximately 1×10⁸ cells were washed with 10 mL of selection buffer, and a supernatant was then removed from the reaction solution. The remaining solution was washed with 1 mL of selection buffer, and it was then suspended in 950 μL of selection buffer. To this cell suspension, 50 μL of the antigen magnetic beads prepared in 17-1 above was added, and the obtained mixture was then reacted at 4° C. for 30 minutes, while being stirred by rotation. Thereafter, using KingFisher mL (Thermo, 5400050), the reaction solution was washed with 0.5 mL of selection buffer three times. Thereafter, the recovered cells were suspended in 20 mL of medium, the suspension was then plated on a 9-cm dish, and it was then cultured in a CO₂ incubator for 7 days.

### 39-3. Screening by Flow Cytometry

The cell culture solution obtained in 39-2 above was recovered, and 3×10⁶ cells were then fractionated into a 1.5-mL tube. The cells were centrifuged to remove a supernatant, and the resulting cells were then washed with 1.5 mL of PBS to remove a supernatant. Using EZ-Link (registered trademark) NHS-PEG4-Biotinylation Kit (PIERCE, 21455), a biotin-labeled Plexin A4 protein was prepared, and 300 μL of primary staining solution (2.5 μg/mL biotin-labeled Plexin A4 protein solution was then added to the protein. The obtained mixture was left at rest at 4° C. under light-shielded conditions for 60 minutes. Thereafter, a supernatant was removed by centrifugation, and the resultant was then washed with 500 μL of PBS twice. After that, 300 μL of secondary staining solution, in which Goat Anti-Human IgG gamma chain specific R-FITC conjugated (Southern biotech, A2040-02) and Streptavidin PE conjugated (eBioscience, 12-4317-87) had been each diluted with buffer 1000 times and 500 times, respectively, was added to the resultant, and the obtained mixture was then left at rest at 4° C. under light-shielded conditions for 60 minutes. Thereafter, a supernatant was removed by centrifugation, and the resultant was then washed with 500 μL of PBS twice. The resultant was suspended in 500 μL of FACS buffer (containing PI solution (Invitrogen, P3566), which had been 1000 times diluted with PBS). This sample was subjected to a flow cytometric analysis. The results are shown in FIG. 36. A cell population, in which the signals of both Plexin A4 protein and IgGγ were high, was discovered in the P5 area.

### 39-4. Screening by Antigen-Immobilized ELISA Method

The cell culture supernatant obtained in 39-3 above was recovered, and a clone reacting with a Plexin A4 antigen was screened by an antigen-immobilized ELISA method using such a Plexin A4 antigen.

First, 20 μL of solution comprising a 2.5 μg/mL Plexin A4 antigen protein and a negative control that was a trypsin inhibitor (Sigma, T6522), streptavidin (Nacalai Tesque Inc., 32243) or ovalbumin (Sigma, A5503) was dispensed in an immuno 384-well plate Maxisorp (Nunc, 464718), and it was then reacted at 4° C. overnight, so that it was immobilized on the plate. Thereafter, the reaction mixture was washed with a washing solution (PBS containing 0.05% Tween 20) three times, and 45 μL of blocking solution (PBS containing 1% BSA) was then added thereto. The obtained mixture was reacted at room temperature for 1 hour. The resultant was washed with a washing solution three times, 25 μL of a measurement sample was then added thereto, and the obtained mixture was then reacted at room temperature for 1 hour. The resultant was washed with a washing solution three times, 25 μL of Goat anti-Human IgG-Fc HRP-conjugated (Bethyl, A80-104P), which had been 2000 times diluted with PBS buffer, was then added thereto, and the obtained mixture was then reacted for 1 hour. The resultant was washed with a washing solution three times, 25 μL of TMB+(Dako, S159985) was then added thereto, and the obtained mixture was then subjected to a coloring reaction for 5 minutes. Thereafter, 25 μL of 1 M sulfuric acid was added to the reaction mixture, so as to terminate the reaction. Using Infinite M1000 (TECAN), the absorbance at 450 nm was measured. The results are shown in FIG. 37. It was demonstrated that an antibody specifically reacting with Plexin A4 was generated in almost all of the clones.

According to screening in which the flow cytometry described in 39-3 above was combined with the antigen-immobilized ELISA method described in 39-4 above, a positive clone specifically binding to Plexin A4 could be identified. In addition to the case of B7-3, even in selections in which the L15/H30 cell line #11-6 and the L30/H30 cell line 7-7-48-10 were used, a positive clone specifically binding to Plexin A4 could be identified.

### 40. Confirmation of Expression of Anti-Plexin A4 Antibody

### 40-1. Analysis by ELISA

Among the positive clones identified in 39-5 above, regarding the positive clones #62 and #20, the concentrations of chicken IgM and human IgG₁ secreted into the culture supernatant were measured. Each cell line was suspended in a medium containing no chicken serum to result in 4×10⁵ cells/mL, and 1 mL each of the suspension was plated on a 24-well plate. The cells were cultured for 3 days, and the culture solution was recovered and was then filtrated through a 0.22-μm filter. Thereafter, the resulting cells were used in the measurement.

The method for measuring IgG is as follows. 100 μL of Goat anti-Human IgG-Fc affinity purified (Bethyl, A80-104A), which had been adjusted to 1.0 μg/mL by dilution with PBS, was dispensed in F96 Maxisorp Nunc Immunoplate (Nunc, 464718), and it was then reacted at 4° C. for 1 hour, so that it was immobilized on the plate. Thereafter, the reaction mixture was washed with a washing solution (PBS containing 0.05% Tween 20) five times, and 200 μL of blocking solution (PBS containing 1% BSA) was then added thereto. The obtained mixture was reacted at room temperature for 95 minutes. The resultant was washed with a washing solution five times, and 100 μL of a measurement sample or Human IgG1 Lambda-UNLB (Southern Biotech, 0151L-01) serving as a standard substance was then added thereto. The obtained mixture was reacted at room temperature for 1 hour. The resultant was washed with a washing solution five times, and 100 μL of Goat anti-Human IgG-Fc HRP conjugated (Bethyl, A80-104P), which had been 1000 times diluted with PBS, was then added thereto. The obtained mixture was reacted at room temperature for 1 hour. The resultant was washed with a washing solution three times, and 100 μL of TMB+(Dako, S159985) was then added thereto. The obtained mixture was subjected to a coloring reaction at room temperature for 3 minutes, and 20 μL of 1 M sulfuric acid was then added thereto, so as to terminate the reaction. Using Infinite M1000 (TECAN), the absorbance at 450 nm was measured.

The method for measuring IgM is as follows. 100 μL of Goat anti-chicken IgM (Bethyl, A30-102A), which had been diluted with PBS to 1.0 μg/mL, was dispensed in F96 Maxisorp Nunc Immunoplate (Nunc, 439454), and it was then reacted at 4° C. overnight, so that it was immobilized on the plate. Thereafter, the reaction mixture was washed with a washing solution (PBS containing 0.05% Tween 20) five times, and 200 μL of blocking solution (PBS containing 1% BSA) was then added thereto. The obtained mixture was reacted at room temperature for 95 minutes. The resultant was washed with a washing solution five times, and 100 μL of chicken serum (GIBCO, 16110) serving as a measurement sample or a standard substance was added thereto. The obtained mixture was reacted at room temperature for 1 hour. The resultant was washed with a washing solution five times, and 100 of Goat anti-chicken IgM HRP conjugated (Bethyl, A30-102P), which had been 5000 times diluted with PBS containing 1% BSA and 0.05% Tween 20, was then added thereto. The obtained mixture was reacted at room temperature for 1 hour. The resultant was washed with a washing solution five times, and 100 μL of TMB+(Dako, S159985) was then added thereto. The obtained mixture was subjected to a coloring reaction at room temperature for 3 minutes, and 100 μL of 1 N sulfuric acid was then added thereto, so as to terminate the reaction. Using Infinite M1000 (TECAN), the absorbance at 450 nm was measured.

The measurement results are shown in Table 37. It was confirmed that human IgG₁ was secreted from the clone #20 and the clone #62, and that the secretion level of chicken IgM was extremely small

TABLE 37

| Clone name | IgG (μg/mL) | IgM (μg/mL) |
|---|---|---|
| #62 | 5.79 | 0.186 |
| #20 | 1.54 | 0.231 |

### 40-2. Analysis by Western Blotting

Antibodies secreted from the positive clones #62 and #20, which produce the antigen-reactive antibody obtained in 17-4 above, were analyzed according to Western blotting.

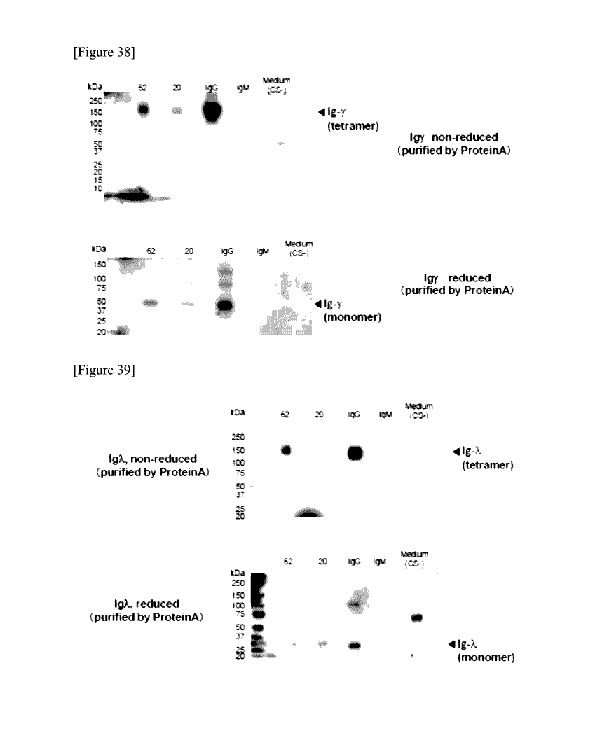

Each cell line was suspended in a medium containing no chicken serum to result in 4×10⁵ cells/mL, and 1 mL each of the suspension was plated on a 24-well plate. The cells were cultured for 3 days, and the culture solution containing the cell line was recovered and was then filtrated through a 0.22-μm filter (Millipore, SLGV J13 SL). For detection of Igγ and Igλ, a sample purified with a Protein A column was used, and for detection of IgM, a sample before purification was used. To a sample used in detection under reduction conditions (reduction sample), Tris-SDS-β-mercaptoethanol sample treatment solution (COSMO BIO Co., Ltd., 423437) was added in an equal amount of the aforementioned sample. On the other hand, to a sample used in detection under non-reduction conditions (non-reduction sample), Tris-SDS sample treatment solution (COSMO BIO Co., Ltd., 423420) was added in an equal amount of the aforementioned sample. Thereafter, the reduction sample was reacted at 98° C. for 3 minutes, and the non-reduction sample was reacted at 37° C. for 30 minutes. Thereafter, the sample was electrophoresed on a 4% to 20% polyacrylamide gel (COSMO BIO Co., Ltd., 414879), and it was then transcribed on a nylon membrane. After that, it was blocked with a blocking buffer (PBS containing 1% BSA), and was then reacted with a primary antibody (Goat Anti-Human IgG-Fc Fragment Ab-HRP (Bethyl, A80-104P), Goat Anti-Human IgG Lambda-HRP (Southern Biotech, 2070-05), and Goat Anti-chicken IgM-HRP (Bethyl, A30-102P). The resultant was washed with PBS containing 0.1% Tween 20 three times, and then, chicken IgM, human Igγ (heavy chain), and human Igλ (light chain) were detected by chemiluminescence using ECL plus (GE Healthcare, RPN2132). The results are shown in FIG. 38 to FIG. 40. According to Western blotting using an anti-Igγ antibody, a band of 55 kDa was detected under reduction conditions, and a band of approximately 160 kDa was detected under non-reduction conditions (FIG. 38). According to Western blotting using an anti-Igλ antibody, a band of 25 kDa was detected under reduction conditions, and a band of approximately 160 kDa was detected under non-reduction conditions (FIG. 39). Moreover, according to Western blotting using an anti-IgM antibody, no signals were detected (FIG. 40).

From these results, it was confirmed that the antibody produced by the obtained positive clone is a full-length human antibody.

### Antibody Selection 2

### 41. Preparation of Antigen

Semaphorin 3A (Sema3A) was selected as an antigen protein. Sema3A is a molecule belonging to the Sema3 family, and has been known as a factor for controlling axon extension in the sensory nerve or the sympathetic nerve.

As an antigen, His-AP-hSema3A (SEQ ID NO: 183), in which a His tag and human alkaline phosphatase (AP) were added to the N-terminus of Sema3A, was used. A cell line HEK293 stably expressing this protein was provided by Department of Molecular Pharmacology & Neurobiology, Yokohama City University, Graduate School of Medicine.

The expression was carried out at 4 L-scale, and the culture was carried out for 4 days. Utilizing His tags, a protein of interest in the culture supernatant was purified. 5 mL of HisTrap EXCEL (GE Healthcare, 17-3712-06) was equilibrated with Solution A (20 mM Na-phosphate, 150 mM NaCl, 20 mM Imidazole (pH 7.5)), a culture supernatant was then loaded thereon, and it was then washed with Solution A. Thereafter, elution was carried out by gradient elution in which Solution A was linearly replaced with Solution B (20 mM Na-phosphate, 150 mM NaCl, 500 mM Imidazole (pH 7.5)) at 25 column volumes. The resultant was monitored with the absorbance at 280 nm, and a fraction corresponding to the peak was then recovered. The molecular weight of the recovered protein was measured by SDS-PAGE and CBB staining, and it was then confirmed that the obtained molecular weight corresponded to the molecular weight of the protein of interest.

Using an ultrafiltration membrane (Amicon, UFC0801008), approximately 80 mL of the recovered fraction was concentrated to a volume of 1.5 mL, and then, using Hi Load 16/600 Superdex 200 pg (GE Healthcare, 28-9893-35), the fraction was purified by gel filtration chromatography. The column was equilibrated with an elution buffer (50 mM Na-phosphate, 300 mM NaCl, 400 mM Arginine, pH 7.0), and the recovered fraction was then loaded thereon, followed by elution with the elution buffer. The resultant was monitored with the absorbance at 280 nm, and a fraction corresponding to the peak was then recovered. The molecular weight of the recovered protein was measured by SDS-PAGE and CBB staining. Subsequently, in order to remove arginine from the eluant, the second gel filtration chromatography was carried out with D-PBS(−). Similarly, the resultant was monitored with the absorbance at 280 nm, and a fraction corresponding to the peak was then recovered. The molecular weight of the recovered protein was measured by SDS-PAGE and CBB staining.

### 42. Selection of Antibody

### 42-1. Production of Antigen Magnetic Beads

Using Dynabeads M-270 Carboxylic Acid (Life Technologies, 14305D), Dynabeads M-270 Epoxy (Life Technologies, 14301), and Dynabeads His-Tag Isolation & Pulldown (Life Technologies, 10103D) as magnetic beads, these beads were allowed to bind to the antigen in accordance with the instruction manual. At this time, MPC-S(Life Technologies, DBA13346) was used as a magnetic stand. Regarding Dynabeads M-270 Carboxylic Acid, 40 μL of beads were washed with 80 μL of 25 mM IVIES (pH 5.0) three times, and 40 μL of 50 mg/mL NHS solution and 40 μL of 50 mg/mL EDC solution were added to the resulting beads. The obtained mixture was reacted at room temperature for 30 minutes, and the reaction solution was then washed with 80 μL of 25 mM IVIES (pH 5.0) twice. A supernatant was removed from this magnetic beads suspension. For positive selection, 50.6 μL of His-AP-hSema3A protein solution, which had been adjusted to 0.475 mg/ml with 25 mM MES (pH 5.0), was added to the above-obtained solution to make a suspension. For negative selection, PBS was used instead of the antigen solution, and 50.6 μL of the solution diluted with 25 mM IVIES (pH 5.0) was added to the above-obtained solution to make a suspension. The obtained suspension was reacted at 4° C. overnight, while being subjected to rotary stirring. Thereafter, a supernatant was removed from the reaction solution, and 80 μL of quenching buffer (50 mM Tris-HCl) was then added thereto, followed by the reaction of the mixture at room temperature for 15 minutes while being subjected to rotary stirring. Regarding Dynabeads M-270 Epoxy, 40 μL of beads were washed with 80 μL of 100 mM Na-phosphate buffer three times. Thereafter, for positive selection, 53.3 μL of His-AP-hSema3A protein solution adjusted to 0.45 mg/ml with a 100 mM Na-phosphate buffer and 26.7 μL of 100 mM Na-phosphate buffer containing 3 M ammonium sulfate were added to the above-obtained beads to make a suspension, and for negative selection, PBS was used instead of the antigen solution, and 53.3 μL of the solution diluted with 25 mM IVIES (pH 5.0) and 26.7 μL of 100 mM Na-phosphate buffer containing 3 M ammonium sulfate were added to the above-obtained beads to make a suspension. The obtained suspension was reacted at 37° C. overnight, while being subjected to rotary stirring. Regarding Dynabeads His-Tag Isolation & Pulldown, 8 μL of beads were washed with 80 μL of PBS three times. Thereafter, for positive selection, 101.9 μL of His-AP-hSema3A protein solution having an antigen concentration of 4.49 μM was added to the above-obtained beads to make a suspension, and for negative selection, 101.9 μL of PBS was used instead of the antigen solution, and it was then added to the above-obtained beads to make a suspension. The obtained suspension was reacted at 4° C. for 10 minutes, while being subjected to rotary stirring. After the removal of a supernatant, the three types of beads were collectively suspended in 1 mL of selection buffer (PBS containing 1% BSA), and the obtained suspension was then washed with the same buffer as mentioned above three times. Thereafter, the resultant was suspended in 200 μL of selection buffer, and was then subjected to selection.

### 42-2. Selection by Antigen Magnetic Beads

An L15/H15 cell line, B7-3, was cultured in a medium containing 2.5 ng/mL TSA for 86 days. Thereafter, approximately 1.5×10⁷ cells were washed with 10 mL of selection buffer, and a supernatant was then removed from the reaction solution. The remaining solution was washed with 1 mL of selection buffer, and it was then suspended in 950 μL of selection buffer. To this cell suspension, 50 μL of the antigen magnetic beads for negative selection prepared in 39-1 above was added, and the obtained mixture was then reacted at 4° C. for 30 minutes, while being stirred by rotation. Thereafter, using KingFisher mL (Thermo, 5400050), the magnetic beads were removed. Subsequently, to this cell suspension, 50 μL of the antigen magnetic beads for positive selection was added, and the obtained mixture was then reacted at 4° C. for 30 minutes, while being stirred by rotation. Thereafter, using KingFisher mL (Thermo, 5400050), the reaction solution was washed with 1.7 mL of selection buffer three times. Thereafter, the recovered cells were suspended in 20 mL of medium, the suspension was then plated on a 96-well plate, and it was then cultured in a CO₂ incubator.

### 42-3. Primary Screening

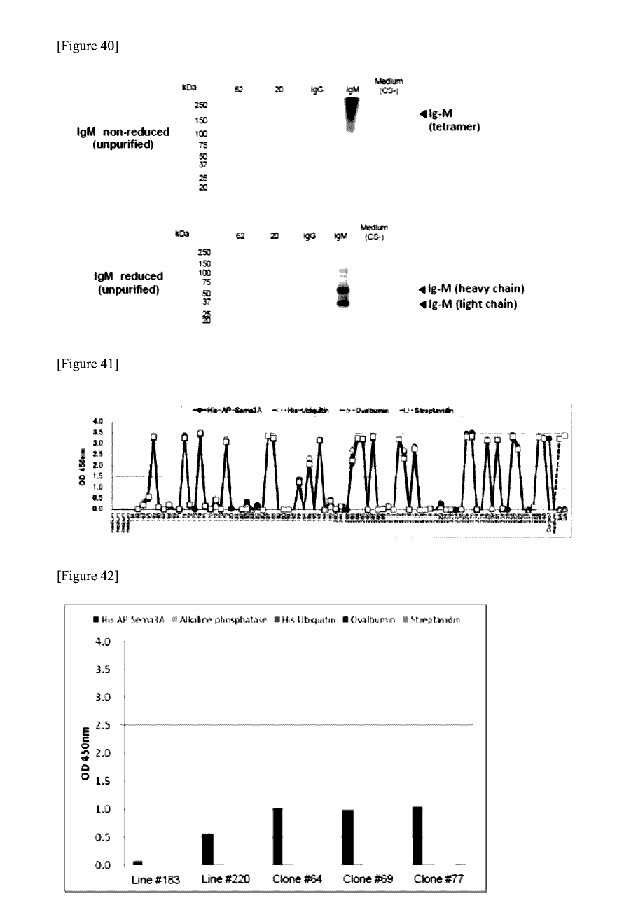

The cell culture solution obtained in 42-2 above was recovered, and a clone specifically reacting with a target antigen was screened based on the antigen reactivity of secretory IgG in antigen-immobilized ELISA. 20 μL of antigen protein and negative control antigen solution (His-Ubiquitin, Ovalbumin, Streptavidin), which had been adjusted to 2.5 μg/mL with PBS, was dispensed on an immuno 384-well plate Maxisorp (Nunc, 464718), and it was then reacted at 4° C. overnight, so that it was immobilized thereon. The resultant was washed with a washing solution (PBS containing 0.05% Tween 20) five times, and 45 μL of a blocking solution (PBS containing 1% BSA) was then added thereto. The obtained mixture was reacted at room temperature for 115 minutes. The reaction mixture was washed with a washing solution five times, and 25 μL of culture supernatant was then added thereto. The obtained mixture was reacted at room temperature 130 minutes. Thereafter, the resultant was washed with a washing solution five times, and 25 μL of Goat anti-Human IgG-Fc HRP-conjugated, which had been 2000 times diluted with a blocking solution, was added thereto. The obtained mixture was reacted at room temperature 47 minutes. The resultant was washed with a washing solution five times, 25 μL of TMB+(Dako, S159985) was then added thereto, and the obtained mixture was then reacted for 5 minutes. Thereafter, 25 μL of 1 N sulfuric acid was added to the reaction mixture, so as to terminate the reaction, and then, using a microplate reader, the absorbance at 450 nm was measured (FIG. 41). The cell having an absorbance of 0.1 or more and an S/N ratio of 5 or more was determined to be a positive cell, and it was then subjected to secondary screening

### 42-4. Secondary Screening

The cell culture supernatant obtained in 42-3 above was recovered, and it was then subjected to the secondary screening according to antigen-immobilized ELISA based on the method described in 42-3 above. The cell having an absorbance of 0.5 or more and an S/N ratio of 5 or more was determined to be a positive cell, and it was then subjected to monocloning

42-5. Primary Screening after Monocloning

The cell culture supernatant obtained in 42-4 above was recovered, and it was then subjected to screening according to antigen-immobilized ELISA based on the method described in 42-3 above. The cell having an absorbance of 0.2 or more and an S/N ratio of 5 or more was determined to be a positive cell, and it was then subjected to secondary screening.

42-6. Secondary Screening after Monocloning

The cell culture supernatant obtained in 42-5 above was recovered, and it was then subjected to screening according to antigen-immobilized ELISA based on the method described in 42-3 above. Positive clones #64, #69 and #77, which had an absorbance of 0.5 or more and an S/N ratio of 5 or more, were selected (FIG. 42).

### 43. Confirmation of Expression of Anti-his-AP-hSema3A Antibody

### 43-1. Analysis by ELISA

Among the positive clones selected in 42-6 above, regarding the positive clone #64, the concentrations of chicken IgM and human IgG₁ secreted into the culture supernatant were measured. The measurement method was as described in 40-1 above.

The measurement results are shown in Table 38. It was confirmed that, as with wild-type DT40, human IgG was secreted from the positive clone #64, and that secretion of chicken IgM was not observed.

TABLE 38

| Clone name | IgG (μg/mL) | Chicken IgM (μg/mL) |
|---|---|---|
| #64 | 2.66 | <0.0005 |

### 43-2. Analysis by Western Blotting

According to Western blotting, an antibody secreted from the positive clone #64 producing an antigen-reactive antibody obtained in 42-6 above was analyzed.

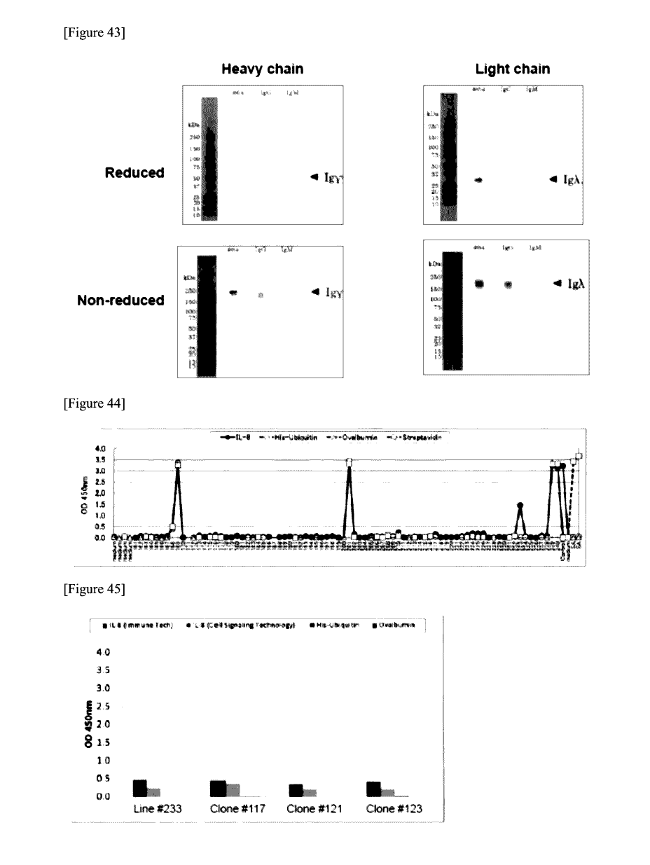

Each cell line was suspended in a medium containing no chicken serum to result in 4×10⁵ cells/mL, and 20 mL of the suspension was plated on a 9-cm dish. The cells were cultured for 4 days, and the culture solution containing the cell line was recovered and was then filtrated through a 0.22-μm filter (Millipore, SLGV J13 SL). For detection of Igγ and Igλ, a sample purified with a Protein A column was used, and for detection of IgM, a sample before purification was used. To a sample used in detection under reduction conditions (reduction sample), Tris-SDS-β-mercaptoethanol sample treatment solution (COSMO BIO Co., Ltd., 423437) was added in an equal amount of the aforementioned sample. On the other hand, to a sample used in detection under non-reduction conditions (non-reduction sample), Tris-SDS sample treatment solution (COSMO BIO Co., Ltd., 423420) was added in an equal amount of the aforementioned sample Thereafter, the reduction sample was reacted at 98° C. for 3 minutes, and the non-reduction sample was reacted at 37° C. for 30 minutes. Thereafter, the sample was electrophoresed on XV PANTERA 5-20% T-HCL 10W (DRC, NXV-275HP), and it was then transcribed on a PVDF membrane. After that, it was blocked with a blocking buffer (TB S containing 5% skimmed milk and 0.1% Tween 20), and was then reacted with a primary antibody (Goat Anti-Human IgG-Fc Fragment Ab-HRP (Bethyl, A80-104P) and Goat Anti-Human IgG Lambda-HRP (Southern Biotech, 2070-05)). The resultant was washed with TBS containing 0.1% Tween 20 three times, and then, chicken IgM, human Igγ (heavy chain), and human Igλ (light chain) were detected by chemiluminescence using Luminata Forte Western HRP substrate (Millipore, WBLUF0100). The results are shown in FIG. 43. According to Western blotting using an anti-Igγ antibody, a band of 55 kDa was detected under reduction conditions (FIG. 43A), and a band of approximately 160 kDa was detected under non-reduction conditions (FIG. 43B). According to Western blotting using an anti-Igλ antibody, a band of 25 kDa was detected under reduction conditions (FIG. 43C), and a band of approximately 160 kDa was detected under non-reduction conditions (FIG. 43D).

From these results, it was confirmed that the antibody produced by the obtained positive clone is a full-length human antibody.

### Antibody Selection 3

### 44. Preparation of Antigen

IL-8 was selected as an antigen protein. IL-8 is one type of chemokine. IL-8 exhibits chemotaxis to neutrophils and T lymphocytes, and has an activity of promoting adhesion of leucocytes to vascular endothelial cells or the functions of neutrophils. Hence, it is considered that IL-8 is associated with inflammatory disease, rheumatoid arthritis and other diseases, which are attended with infiltration of neutrophils.

As antigens, human IL-8 (Immune TECH, IT-401-003P) was used in selection, and two types of human IL-8 (Immune TECH, IT-401-003P, and CELL Signaling Technology, 8921LF) were used in screening.

### 45. Selection of Antibody

### 45-1. Production of Antigen Magnetic Beads

Using Dynabeads M-270 Carboxylic Acid (Life Technologies, 14305D), Dynabeads M-270 Epoxy (Life Technologies, 14301) and Dynabeads His-Tag Isolation & Pulldown (Life Technologies, 10103D) as magnetic beads, these beads were allowed to bind to the antigen in accordance with the instruction manual. At this time, MPC-S(Life Technologies, DBA13346) was used as a magnetic stand. Regarding Dynabeads M-270 Carboxylic Acid, 35 μL of beads were washed with 70 μL of 25 mM IVIES (pH 5.0) three times, and 35 μL of 50 mg/mL NHS solution and 35 μL of 50 mg/mL EDC solution were added to the resulting beads. The obtained mixture was reacted at room temperature for 30 minutes, and the reaction solution was then washed with 70 μL of 25 mM IVIES (pH 5.0) twice. A supernatant was removed from this magnetic beads suspension. For positive selection, 35 μL of IL-8 (Immune TECH, IT-401-003P) protein solution adjusted to 0.6 mg/ml with 25 mM IVIES (pH 5.0) was added to the above-obtained solution to make a suspension. For negative selection, PBS was used instead of the antigen solution, and 35 μL of the solution diluted with 25 mM IVIES (pH 5.0) was added to the above-obtained solution to make a suspension. The obtained suspension was reacted at 4° C. overnight, while being subjected to rotary stirring. Thereafter, a supernatant was removed from the reaction solution, and 70 μL of quenching buffer (50 mM Tris-HCl) was then added thereto, followed by the reaction of the mixture at room temperature for 15 minutes while being subjected to rotary stirring. Regarding Dynabeads M-270 Epoxy, 35 μL of beads were washed with 70 μL of 100 mM Na-phosphate buffer three times. Thereafter, for positive selection, 46.7 μL of IL-8 protein solution adjusted to 0.45 mg/ml with a 100 mM Na-phosphate buffer and 23.3 μL of 100 mM Na-phosphate buffer containing 3 M ammonium sulfate were added to the above-obtained solution to make a suspension, and for negative selection, PBS was used instead of the antigen solution, and 46.7 μL of the solution diluted with 100 mM Na-phosphate buffer and 23.3 μL of 100 mM Na-phosphate buffer containing 3 M ammonium sulfate were added to the above-obtained solution to make a suspension. The obtained suspension was reacted at 37° C. overnight, while being subjected to rotary stirring. Regarding Dynabeads His-Tag Isolation & Pulldown, 7 μL of beads were washed with 70 μL of PBS three times. Thereafter, for positive selection, 70 μL of IL-8 protein solution having an antigen concentration of 5.75 μM was added to the above-obtained solution to make a suspension, and for negative selection, 70 μL of PBS was used instead of the antigen solution, and it was added to the above-obtained solution to make a suspension. The obtained suspension was reacted at 4° C. for 210 minutes, while being subjected to rotary stirring. After the removal of a supernatant, the three types of beads were collectively suspended in 1 mL of a selection buffer (PBS containing 1% BSA), and the obtained suspension was then washed with the same buffer as mentioned above three times. Thereafter, the resultant was suspended in 700 μL of selection buffer, and was then subjected to selection.

### 45-2. Selection by Antigen Magnetic Beads

An L30/H15f15f15f cell line, 2-1-3, was cultured in a medium containing 2.5 ng/mL TSA for 45 days. Thereafter, approximately 1.5×10⁷ cells were washed with 10 mL of selection buffer, and a supernatant was then removed from the reaction solution. The remaining cells were washed with 10 mL of selection buffer, and it was then suspended in 950 μL of selection buffer. To this cell suspension, 50 μL of the antigen magnetic beads for negative selection prepared in 39-1 above was added, and the obtained mixture was then reacted at 4° C. for 30 minutes, while being stirred by rotation. Thereafter, using KingFisher mL (Thermo, 5400050), the magnetic beads were removed. Subsequently, to this cell suspension, 50 μL of the antigen magnetic beads for positive selection was added, and the obtained mixture was then reacted at 4° C. for 30 minutes, while being stirred by rotation. Thereafter, using KingFisher mL (Thermo, 5400050), the reaction solution was washed with 1.7 mL of selection buffer three times. Thereafter, the recovered cells were suspended in 20 mL of medium, the suspension was then plated on a 96-well plate, and it was then cultured in a CO₂ incubator.

### 45-3. Screening and Identification of Positive Clones

In the same matter as described in 42-3 to 42-6 above, two times of screening, monocloning, and two times of screening were carried out to obtain positive clone #117, #121 and #123. The results of the primary screening after completion of the selection are shown in FIG. 44, and the results of the secondary screening after completion of the monocloning are shown in FIG. 45.

### 46. Confirmation of Expression of Anti-IL-8 Antibody

### 46-1. Analysis by ELISA

Regarding the positive clones #117 and #121 generating antigen-reactive antibodies obtained in 45-3 above, the concentrations of chicken IgM and human IgG₁ secreted into the culture supernatant were measured. The measurement method was as described in 40-1 above.

The measurement results are shown in Table 39. It was confirmed that, as with wild-type DT40, human IgG was secreted from the positive clones #117 and #121, and that secretion of chicken IgM was not observed.

TABLE 39

| Clone name | IgG (μg/mL) | IgM (μg/mL) |
|---|---|---|
| #117 | 3.65 | <0.041 |
| #121 | 3.7 | <0.041 |

### 46-2. Analysis by Western Blotting

According to Western blotting, antibodies secreted from the positive clones #117 and #121 were analyzed. The method was as described in 40-2 above.

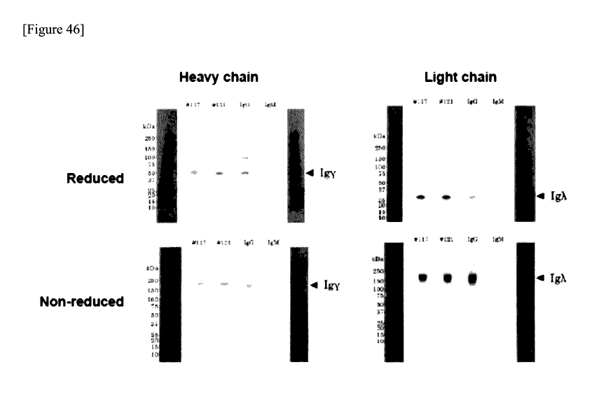

The results are shown in FIG. 46. According to Western blotting using an anti-Igγ antibody, a band of 55 kDa was detected under reduction conditions (FIG. 46A), and a band of approximately 160 kDa was detected under non-reduction conditions (FIG. 46B). According to Western blotting using an anti-Igλ antibody, a band of 25 kDa was detected under reduction conditions (FIG. 46C), and a band of approximately 160 kDa was detected under non-reduction conditions (FIG. 46D).

From these results, it was confirmed that the antibody produced by the obtained positive clone is a full-length human antibody.

### INDUSTRIAL APPLICABILITY

The present invention relates to a method for promptly producing a variety of human antibodies. Considering the importance of antibody drugs, it is anticipated that the technique provided by the present invention will play an extremely important role in the development of biotechnology-based pharmaceutical products exhibiting desired drug effects, in particular, antibody drugs, in the field of future drug discovery and medical services.

### SEQUENCE LISTING

### TPC0130CBS-sequencing_ST25.txt

1. A chicken B cell, in which in chicken antibody light chain gene locus is replaced by the DNA sequence derived from a human antibody light chain variable region and by the DNA sequence derived from a human antibody light chain constant region,
which in the chicken antibody heavy chain gene locus all or a part of the DNA sequence derived from a human antibody heavy chain variable region and of a human antibody heavy chain constant region are inserted, and in which the chicken antibody light chain pseudogene locus is replaced by 25 or more DNA sequences derived from human antibody light chain variable regions,
and in which the chicken antibody heavy chain pseudogene locus is inserted by 30 or more DNA sequences derived from human antibody heavy chain variable regions,
wherein the chicken B cell has the ability to express a human antibody on the cell surface and also to secrete the human antibody into the culture solution.

2. (canceled)

3. (canceled)

4. (canceled)

5. (canceled)

6. The chicken B cell according to claim 1, wherein the DNA sequence derived from the human antibody light chain variable region inserted into the antibody light chain gene locus is at a position upstream from the DNA sequence derived from the human antibody light chain constant region, and the DNA sequence derived from the human antibody heavy chain variable region inserted into the antibody heavy chain gene locus is at a position upstream from the DNA sequence derived from the human antibody heavy chain constant region.

7. (canceled)

8. The chicken B cell according to claim 1, wherein the human antibody heavy chain is a γ chain.

9. The chicken B cell according to claim 1, wherein the human antibody light chain is a γ chain or a κ chain.

10. (canceled)

11. The chicken B cell according to claim 1, which is a DT40 cell.

12. The chicken B cell according to claim 1, which has been subjected to a treatment of relaxing chromatin.

13. The chicken B cell according to claim 12, wherein the treatment of relaxing chromatin is reduction or deletion of the function of histone deacetylase in the chicken B cell.

14. The chicken B cell according to claim 13, wherein the method for reducing or deleting the function of histone deacetylase is reduction or deletion of the expression of a histone deacetylase gene in the chicken B cell.

15. The chicken B cell according to claim 14, wherein the histone deacetylase is HDAC2.

16. The chicken B cell according to claim 13, wherein the method for reducing or deleting the function of histone deacetylase is a treatment with an HDAC inhibitor.

17. An antibody-producing cell library consisting of the chicken B cells according to claim 1.

18. A method for producing a human antibody or a humanized antibody from the chicken B cell according to claim 1, comprising culturing said chicken B cell under culturing conditions suitable for the cell to produce a human antibody or a humanized antibody and obtaining said antibody.

19. Antibodies reacting with various antigens, wherein said antibodies have been produced by the cell library according to claim 17.

20. A kit for producing the chicken B cells according to claim 1.

21. (canceled)

22. The method of claim 18, wherein the antibody is comprised in a pharmaceutical product.
